(19) United States
(12) Patent Application Publication
(10) Pub. No.: US 2025/0262331 A1
(43) Pub. Date: Aug. 21, 2025

(54) COMPOSITION AND METHODS FOR IMPROVING SIGNS AND SYMPTOMS OF OCULAR DISORDERS

(71) Applicant: Azura Ophthalmics Ltd., Tel Aviv (IL)

(72) Inventors: Charles BOSWORTH, Las Vegas, NV (US); Yair ALSTER, Tel Aviv (IL); Omer RAFAELI, Udim (IL); Marc GLEESON, Longueville (AU)

(21) Appl. No.: 19/203,615

(22) Filed: May 9, 2025

Related U.S. Application Data
(63) Continuation of application No. PCT/US2023/000680, filed on Nov. 9, 2023.
(60) Provisional application No. 63/424,768, filed on Nov. 11, 2022.
(60) Provisional application No. 63/459,545, filed on Apr. 14, 2023.

(51) Int. Cl. A61K 49/00; A61K 9/00; A61K 33/04

(52) U.S. Cl. CPC A61K 49/0004; A61K 9/0048; A61K 33/04

(57) ABSTRACT

Provided herein are methods for improving signs and symptoms of ocular indications and treating ocular disorders using a keratolytic agent.

### CROSS-REFERENCE

This application is a continuation of International Application No. PCT/IB 2023/000680, filed internationally on Nov. 9, 2023, which claims the benefit of U.S. Provisional Application No. 63/424,768, filed on Nov. 11, 2022, and U.S. Provisional Application No. 63/459,545, filed on Apr. 14, 2023, which are each hereby incorporated by reference in their entirety herein.

### BACKGROUND OF THE INVENTION

Meibomian gland dysfunction (MGD) is a common disease in which a patient's meibomian glands produce insufficient meibum that would otherwise prevent dryness of the eye. M eibum is an oily substance comprised largely of lipids that helps maintain the eye's tear film by preventing evaporation or pooling. In MGD, obstruction of the meibomian glands prevents the secretion of meibum, therefore causing dry eyes, pain, and blepharitis in many patients. In order to alleviate the obstruction of the meibomian glands and restore lipid production (e.g., meibum production), ophthalmic preparations of selenium disulfide may be applied to the eyelid, eyelid margin, ocular surface, or surrounding tissues.

Adverse effects (e.g., pain, local irritation, inflammation, redness, keratosis, etc.) have been identified in applications of selenium disulfide in or around the eye, particularly when high concentrations of selenium disulfide are utilized. In certain instances, administration of therapeutically effective concentrations of selenium disulfide (e.g., to the eyelid margin) have been found to minimize adverse effects, while also providing therapeutic benefit.

### SUMMARY OF THE INVENTION

Described herein are the methods of use for an ophthalmic preparation containing keratolytic agents, such as selenium disulfide, to improve an objective measure (e.g., sign or other morphological characteristic or biomarker) and/or symptoms of ocular indications. In some embodiments, use of the ophthalmic preparation containing keratolytic agents (e.g., initially only) provides improvement in the objective measure, such as a sign or other morphological characteristic or biomarker of an ocular indication described herein. For example, while the sign(s) of the ocular indication(s) can rapidly improve, such as within day 14 of an initial dose of an agent described herein, the symptom(s) of the ocular indication(s) can be delayed, such as by several months of the initial dose of an agent described herein. In some instances, such as at the onset of treatment (e.g., within the first month or so of treatment) the symptoms may remain the same, or even deteriorate. In some instances, continuing use of a keratolytic agent described herein, such as for a period of time sufficient to provide a morphological change to one or more meibomian gland of the individual, can provide significant improvement to one or more symptom of the ocular indication (e.g., as a result of continued improvement of the objective measure). In some instances, improvements in objective measures and symptoms occur within about three months of one-weekly administration of a keratolytic agent described herein. Moreover, several measures of the objective measures and symptoms are identified herein. Evaluation of such measures can be used to help determine dosing regimens, frequency, treatment success, or the like of keratolytic agents for ocular use. Additionally, in some instances, a significant (e.g., ≥5%) reduction in (e.g., treatment emergent) adverse events is observed when administration of a keratolytic described herein is continued for an extended period of time, such as at least four months (e.g., four to six months).

Provided in some embodiments herein is a method for treating an ocular disorder in an individual, the method comprising providing a keratolytic agent (e.g., selenium disulfide) to or around the eye of the individual until an objective measure and/or symptom of the ocular disorder are improved.

Provided in some embodiments herein is a method for treating an ocular disorder in an individual, the method comprising:
i. evaluating an objective measure for an ocular indication to determine an initial objective score;
ii. providing a keratolytic agent (e.g., selenium sulfide) to or around the eye of the individual;
iii. evaluating the objective measure to determine a second objective score after keratolytic agent (e.g., selenium sulfide) has been provided to or around the eye of the individual at least one time;
iv. determining if the second objective score is an improvement over the initial objective score; and
v. if the second objective score is an improvement over the initial objective score, providing keratolytic agent (e.g., selenium sulfide) to or around the eye of the individual.

In some embodiments, the method further comprises evaluating a symptom for the ocular indication to determine an initial symptom score.

In some embodiments, the method further comprises evaluating a symptom to determine a second symptom score.

In some embodiments, the method further comprises evaluating a symptom to determine a second symptom score after observing an improvement in the second objective score over the initial objective score.

In some embodiments, such as if the second symptom score is an improvement over the initial symptom score, the method further comprises provide keratolytic agent (e.g., selenium sulfide) to or around the eye of the individual.

In some embodiments, such as if the second symptom score is not an improvement over the initial symptom score, the method further comprises providing keratolytic agent (e.g., selenium sulfide) to or around the eye of the individual until the second symptom score is an improvement over the initial symptom score.

Provided in some embodiments herein is a method for treating an ocular disorder in an individual, the method comprising:
i. evaluating an objective measure for an ocular indication to determine an initial objective score;
ii. evaluating a symptom for the ocular indication to determine an initial symptom score;
iii. providing a keratolytic agent (e.g., selenium sulfide) to or around the eye of the individual;
iv. evaluating the objective measure to determine a second objective score after keratolytic agent (e.g., selenium sulfide) has been provided to or around the eye of the individual at least one time;
v. determining if the second objective score is an improvement over the initial objective score;
vi. if the second objective score is an improvement over the initial objective score, providing keratolytic agent (e.g., selenium sulfide) to or around the eye of the individual;
vii. evaluating the symptom or initial symptom score to determine a second symptom score; and
viii. if the second symptom score is an improvement over the initial symptom score, providing keratolytic agent (e.g., selenium sulfide) to or around the eye of the individual.

In some embodiments, the initial objective score is determined before providing a keratolytic agent (e.g., selenium sulfide) to or around the eye of the individual. In some embodiments, the initial objective score is a baseline measurement of the objective measure. In some embodiments, improvement in symptom or objective measure is determined by comparing a second score (e.g., the second objective score or the second symptom score) to a baseline measurement.

In some embodiments, improvement in a symptom or objective measure is determined by comparing a second score (e.g., the second objective score or the second symptom score) to a control (e.g., vehicle) measurement.

In some embodiments, the second objective score is compared to a first objective score (e.g., the initial objective score) and determined after providing the keratolytic agent (e.g., selenium sulfide) to or around the eye of the individual.

In some embodiments, the second objective score is an improvement over the initial objective score and the improvement is determined after providing the keratolytic agent (e.g., selenium sulfide) to or around the eye of the individual.

In some embodiments, the second objective score is not an improvement over the initial objective score and the second objective score is determined after providing the keratolytic agent (e.g., selenium sulfide) to or around the eye of the individual.

In some embodiments, such as if the second objective score is not an improvement over the initial objective score, the method comprises providing keratolytic agent (e.g., selenium sulfide) to or around the eye of the individual (e.g., until the second objective score is an improvement over the initial objective score).

In some embodiments, a (significant) improvement in the initial objective score is observed within about 5 months or less (e.g., 3 months or less or 1.5 months or less) after providing the keratolytic agent (e.g., selenium sulfide) to or around the eye of the individual. In some embodiments, a (significant) improvement in the initial objective score is observed within about 3 months or less after providing the keratolytic agent (e.g., selenium sulfide) to or around the eye of the individual. In some embodiments, a (significant) improvement in the initial objective score is observed about 1.5 months after providing the keratolytic agent (e.g., selenium sulfide) to or around the eye of the individual. In some embodiments, a (significant) improvement in the initial objective score is observed about 14 days after providing the keratolytic agent (e.g., selenium sulfide) to or around the eye of the individual. In some embodiments, the individual has an improvement in the initial objective score with continued use of the keratolytic agent (e.g., selenium sulfide), such as continued use through 6-months.

In some embodiments, the objective measure is (of) a morphology or a biomarker.

In some embodiments, the morphology or biomarker is clogged and/or blocked pores, closed meibomian glands, meibum color, meibum quality, meibum quantity, tear open time, lid swelling, lid hyperemia, or conjunctival hyperemia.

In some embodiments, a (significant) improvement in opening meibomian glands of the individual is observed after providing the keratolytic agent (e.g., selenium sulfide) to or around the eye of the individual.

In some embodiments, a (significant) improvement in meibum quality and/or quantity of the individual is observed after providing the keratolytic agent (e.g., selenium sulfide) to or around the eye of the individual.

In some embodiments, a (significant) improvement in opening meibomian glands, meibum quality, and/or meibum quantity of the individual is observed within about 5 months or less (e.g., 3 months or less or 1.5 months or less) after providing the keratolytic agent (e.g., selenium sulfide) to or around the eye of the individual. In some embodiments, the individual has an improvement in opening meibomian glands, meibum quality, and/or meibum quantity with continued use of the keratolytic agent (e.g., selenium sulfide), such as continued use through 6-months.

In some embodiments, the biomarker is meibomian glands yielding liquid secretion score (MGYLS), total ocular surface disease index (OSDI) score, standard patient evaluation of eye dryness (SPEED), meibomian gland score (MGS), or tear breakup time (TBUT).

In some embodiments, the symptom score is (determined by) a (average) visual analogue scale (VAS) (e.g., pain VAS, photophobia VAS, burning/stinging VAS, itching VAS, foreign body VAS, or eye discomfort VAS), SPEED, or contact lens dry eye questionnaire-8 (CLDQ8).

In some embodiments, a (significant) improvement in the initial symptom score is observed within about 5 months or less (e.g., 3 months or less or 1.5 months or less) after providing the keratolytic agent (e.g., selenium sulfide) to or around the eye of the individual.

In some embodiments, the symptom (being evaluated) is eye dryness, pain, photophobia, burning, stinging, itching, grittiness (e.g., feeling like a foreign body is in the eye), or eye discomfort.

In some embodiments, a (significant) improvement in the initial objective score is observed before a (significant) improvement in the symptom score. In some embodiments, a (significant) improvement in the symptom score is observed several weeks after a (significant) improvement in the initial objective score is observed.

In some embodiments, the symptom or symptom score (initially) gets worse for a period of time (e.g., 14 days, 1.5 months) after providing the keratolytic agent (e.g., selenium sulfide) to or around the eye of the individual.

In some embodiments, a (significant) improvement in the symptom score and a (significant) improvement in the initial objective score is observed about 3 months after providing the keratolytic agent (e.g., selenium sulfide) to or around the eye of the individual. In some embodiments, the individual has an improvement in the symptom score and the initial objective score with continued use of the keratolytic agent (e.g., selenium sulfide), such as continued use through 6-months.

In some embodiments, the keratolytic agent (e.g., selenium sulfide) is provided to or around the eye of the individual at least until the second objective score is an improvement over the initial objective score and/or that the second symptom score is an improvement over the initial symptom score.

In some embodiments, providing the keratolytic agent (e.g., selenium sulfide) to or around the eye of the individual is maintained after observing that the second objective score is an improvement over the initial objective score and/or that the second symptom score is an improvement over the initial symptom score.

Provided in some embodiments herein is a method for treating an ocular disorder in an individual, the method comprising:
i. providing a keratolytic agent (e.g., selenium sulfide) to or around the eye of the individual for a first period of time, over which first period of time (e.g., 1-4 weeks) improvement in an objective measure of the ocular disorder is achieved;
ii. providing keratolytic agent (e.g., selenium sulfide) to or around the eye of the individual for a second period of time, over which second period of time (e.g., 2-26 weeks) improvement of a symptom of the ocular disorder is achieved; and
iii. providing keratolytic agent (e.g., selenium sulfide) to or around the eye of the individual, after which time (e.g., 1 month or more), improvement in the objective measure and/or symptom is maintained.

In some embodiments, the objective measure (of the ocular disorder) and/or the symptom (of the ocular disorder) remains the same or gets worse, such as during (a portion of) the first period and/or the second period.

In some embodiments, the objective measure (of the ocular disorder) and/or the symptom (of the ocular disorder) improves (significantly), such as during or after the first period of time and/or the second period of time.

In some embodiments, the keratolytic agent (e.g., selenium sulfide) is administered to the individual monthly, bi-weekly, or once-weekly.

In some embodiments, the keratolytic agent (e.g., selenium sulfide) is administered to the individual once-weekly. In some embodiments, the keratolytic agent (e.g., selenium sulfide) is administered to the individual twice-weekly.

In some embodiments, the keratolytic agent (e.g., selenium sulfide) is administered to the individual for at least one month (e.g., one month or more, two months or more, three months or more, five months or more, or six months or more).

In some embodiments, a keratolytic agent described herein is any keratolytic agent described in U.S. Pat. No. 11,040,062 and U.S. Patent Publication Number 2022/0347207, each of which are incorporated herein by reference in their entirety, in particular for the keratolytic agents provided therein. In some embodiments, the keratolytic agent is selenium disulfide.

In some embodiments, a composition described herein is any composition described in U.S. Pat. No. 11,040,062 and U.S. Patent Publication Number 2022/0347207, each of which are incorporated herein by reference in their entirety, in particular for the compositions provided therein. In some embodiments, the composition comprises the keratolytic agent (e.g., selenium sulfide) and an anhydrous semi-solid (ophthalmic) base. In some embodiments, anhydrous semi-solid (ophthalmic) base is an oleaginous base. the In some embodiments, the composition comprises the keratolytic agent (e.g., selenium sulfide) and an oleaginous base. In some embodiments, the oleaginous base is selected from the group consisting of petrol atum, white petrolatum, petroleum, white petroleum, mineral oil, and vegetable oil. In some embodiments, the oleaginous base is petrolatum. In some embodiments, the oleaginous base is white petrolatum. In some embodiments, the composition comprises about 5 milligrams (mg)/gram (g) of selenium disulfide. In some embodiments, the composition comprises about 0.5% of selenium disulfide.

In some embodiments, a composition described herein comprising a keratolytic agent described herein (e.g., selenium sulfide) is administered to the individual before sleeping (e.g., before going to sleep). In some embodiments, a composition described herein comprising a keratolytic agent described herein (e.g., selenium sulfide) is administered to the individual before bedtime. In some embodiments, a composition described herein comprising a keratolytic agent described herein (e.g., selenium sulfide) is administered to the individual at bedtime. In some embodiments, a composition described herein comprising a keratolytic agent described herein (e.g., selenium sulfide) is administered to the individual about one hour before bedtime. In some embodiments, a composition described herein comprising a keratolytic agent described herein (e.g., selenium sulfide) is administered to the individual at nighttime. In some embodiments, a composition described herein comprising a keratolytic agent described herein (e.g., selenium sulfide) is administered to the individual at dusk.

In some embodiments, a composition comprising a keratolytic agent described herein (e.g., selenium sulfide) is suitable for administration to or around the ocular surface.

In some embodiments, a composition described herein comprising a keratolytic agent described herein (e.g., selenium sulfide) is administered to a lower eyelid of the individual. In some embodiments, a composition described herein comprising a keratolytic agent described herein (e.g., selenium sulfide) is administered to a lower eyelid margin of the individual.

In some embodiments, a composition described herein comprising a keratolytic agent described herein (e.g., selenium sulfide) is dispensed from a device, such as a tube described herein. In some embodiments, the individual dispenses a small amount of the composition (from the tube) onto a finger (e.g., an index finger). In some embodiments, the individual (e.g., evenly) spreads the composition, such as a thin film of the composition, onto an eyelid (e.g., a lower eyelid), such as onto the lower eyelid margin, using the finger (e.g., the index finger).

In some embodiments, the individual waits or allows at least one day between administration of a composition described herein comprising a keratolytic agent described herein (e.g., selenium sulfide).

In some embodiments, the ocular disorder is ocular itching.

In some embodiments, the ocular disorder is meibomian gland dysfunction (MGD).

In some embodiments, the ocular disorder is dye eye.

Provided in some embodiments herein is a method for treating ocular itching in an individual, the method comprising:
i. evaluating an objective measure for an ocular indication to determine an initial objective score;
ii. providing a keratolytic agent (e.g., selenium sulfide) to or around the eye of the individual;
iii. evaluating the objective measure to determine a second objective score after keratolytic agent (e.g., selenium sulfide) has been provided to or around the eye of the individual at least one time;
iv. determining if the second objective score is an improvement over the initial objective score; and
v. if the second objective score is an improvement over the initial objective score, providing keratolytic agent (e.g., selenium sulfide) to or around the eye of the individual.

Provided in some embodiments herein is a method for treating dry eye in an individual, the method comprising:
i. evaluating an objective measure for an ocular indication to determine an initial objective score;
ii. providing a keratolytic agent (e.g., selenium sulfide) to or around the eye of the individual;
iii. evaluating the objective measure to determine a second objective score after keratolytic agent (e.g., selenium sulfide) has been provided to or around the eye of the individual at least one time;
iv. determining if the second objective score is an improvement over the initial objective score; and
v. if the second objective score is an improvement over the initial objective score, providing keratolytic agent (e.g., selenium sulfide) to or around the eye of the individual.

In some embodiments, the individual has itchy eyes.

In some embodiments, the individual has MGD.

In some embodiments, the individual has dry eye disease (DED).

In some embodiments, the individual has itchy eyes and MGD.

Provided in some embodiments herein is use of a composition described herein, such as a composition comprising a keratolytic agent described herein (e.g., selenium sulfide), for opening meibomian glands in an individual. In some embodiments, the individual has DED. In some embodiments, the individual has MGD. In some embodiments, the individual has MGD and DED. In some embodiments, the individual has blocked meibomian glands.

Provided in some embodiments herein is use of a composition described herein, such as a composition comprising a keratolytic agent described herein (e.g., selenium sulfide), for improving meibum quality in an individual. In some embodiments, the individual has DED. In some embodiments, the individual has MGD. In some embodiments, the individual has MGD and DED. In some embodiments, the individual has blocked meibomian glands.

Provided in some embodiments herein is use of a composition described herein, such as a composition comprising a keratolytic agent described herein (e.g., selenium sulfide), treating signs of an ocular disorder described herein in an individual. In some embodiments, the individual has DED. In some embodiments, the individual has MGD. In some embodiments, the individual has MGD and DED.

Provided in some embodiments herein is use of a composition described herein, such as a composition comprising a keratolytic agent described herein (e.g., selenium sulfide), for improving symptoms of an ocular disorder described herein in an individual. In some embodiments, the individual has DED. In some embodiments, the individual has MGD. In some embodiments, the individual has MGD and DED.

Provided in some embodiments herein is use of a composition described herein, such as a composition comprising a keratolytic agent described herein (e.g., selenium sulfide), for opening meibomian glands and improving symptoms of DED in an individual described herein, such as an individual who has MGD and/or DED.

Provided in some embodiments herein is use of a composition described herein, such as a composition comprising a keratolytic agent described herein (e.g., selenium sulfide), for improving meibum and improving symptoms of DED in an individual described herein, such as an individual who has MGD and/or DED.

Provided in some embodiments herein is use of a composition described herein, such as a composition comprising a keratolytic agent described herein (e.g., selenium sulfide), for opening meibomian glands, improving meibum, and improving symptoms of DED in an individual described herein, such as an individual who has MGD and/or DED.

Provided in some embodiments herein is use of a composition described herein, such as a composition comprising a keratolytic agent described herein (e.g., selenium sulfide), for treating signs and symptoms of DED in an individual described herein, such as an individual who has MGD and/or DED.

Provided in some embodiments herein is a method for opening (obstructed) meibomian glands in an individual, the method comprising:
i. evaluating an objective measure for an ocular indication (e.g., as measured by meibomian glands yielding liquid secretion score (MGYLS)) to determine an initial objective score;
ii. providing a keratolytic agent (e.g., selenium sulfide) to or around the eye of the individual;
iii. evaluating the objective measure to determine a second objective score after keratolytic agent (e.g., selenium sulfide) has been provided to or around the eye of the individual at least one time;
iv. determining if the second objective score is an improvement over the initial objective score; and
v. if the second objective score is an improvement over the initial objective score, providing keratolytic agent (e.g., selenium sulfide) to or around the eye of the individual.

Provided in some embodiments herein is a method for improving tear stability in an individual, the method comprising:
i. evaluating an objective measure for an ocular indication (e.g., as measured by tear break up time (TBUT)) to determine an initial objective score;
ii. providing a keratolytic agent (e.g., selenium sulfide) to or around the eye of the individual;
iii. evaluating the objective measure to determine a second objective score after keratolytic agent (e.g., selenium sulfide) has been provided to or around the eye of the individual at least one time;
iv. determining if the second objective score is an improvement over the initial objective score; and
v. if the second objective score is an improvement over the initial objective score, providing keratolytic agent (e.g., selenium sulfide) to or around the eye of the individual.

Provided in some embodiments herein is a method for improving a symptom in or around an eye of an individual, the method comprising:
i. evaluating an objective measure for an ocular indication to determine an initial objective score;
ii. evaluating the symptom (e.g., as measured by (total) ocular surface disease index (OSDI) and/or (average) visual analog scale (VAS)) for the ocular indication to determine an initial symptom score;
iii. providing a keratolytic agent (e.g., selenium sulfide) to or around the eye of the individual;
iv. evaluating the objective measure to determine a second objective score after keratolytic agent (e.g., selenium sulfide) has been provided to or around the eye of the individual at least one time;
v. determining if the second objective score is an improvement over the initial objective score;
vi. if the second objective score is an improvement over the initial objective score, providing keratolytic agent (e.g., selenium sulfide) to or around the eye of the individual;
vii. evaluating the symptom or initial symptom score to determine a second symptom score; and
viii. if the second symptom score is an improvement over the initial symptom score, providing keratolytic agent (e.g., selenium sulfide) to or around the eye of the individual.

Provided in some embodiments herein is a method for treating an ocular disorder in an individual, the method comprising:
i. evaluating a measure of TBUT to determine an initial objective score;
ii. evaluating a measure of total OSDI to determine an initial symptom score;
iii. providing a keratolytic agent (e.g., selenium sulfide) to or around the eye of the individual;
iv. evaluating the measure of TBUT to determine a second objective score after keratolytic agent (e.g., selenium sulfide) has been provided to or around the eye of the individual at least one time;
V. determining if the second objective score is an improvement over the initial objective score;
vi. if the second objective score is an improvement over the initial objective score, providing keratolytic agent (e.g., selenium sulfide) to or around the eye of the individual;
vii. evaluating the measure of total OSDI or initial symptom score to determine a second symptom score; and
viii. if the second symptom score is an improvement over the initial symptom score, providing keratolytic agent (e.g., selenium sulfide) to or around the eye of the individual.

In some embodiments, the individual has an improvement in lipid secretion.

In some embodiments, the individual has an improvement in meibomian glands yielding liquid secretion score (MGYLS) (e.g., compared to baseline and/or vehicle). In some embodiments, the individual has an improvement in MGYLS of three or more (e.g., five or more). In some embodiments, the individual has an improvement in MGYLS five months or less (e.g., three months) after receiving the keratolytic agent (e.g., selenium sulfide). In some embodiments, the individual has an improvement in MGYLS with continued use of the keratolytic agent (e.g., selenium sulfide), such as continued use through 6-months.

In some embodiments, the individual has an improvement in total ocular surface disease index (OSDI) score (e.g., compared to baseline and/or vehicle). In some embodiments, the individual has an improvement in total OSDI of fifteen or less (e.g., thirteen or less). In some embodiments, the individual has an improvement in total OSDI five months or less (e.g., three months) after receiving the keratolytic agent (e.g., selenium sulfide). In some embodiments, the individual has an improvement in total OSDI score with continued use of the keratolytic agent (e.g., selenium sulfide), such as continued use through 6-months.

In some embodiments, the individual has an improvement in standard patient evaluation of eye dryness (SPEED) (e.g., compared to baseline and/or vehicle). In some embodiments, the individual has an improvement from baseline and/or vehicle in SPEED five months or less (e.g., three months) after receiving the keratolytic agent (e.g., selenium sulfide). In some embodiments, the individual has an improvement in SPEED with continued use of the keratolytic agent (e.g., selenium sulfide), such as continued use through 6-months.

In some embodiments, the individual has an improvement in average visual analogue scale (VAS) (e.g., compared to baseline and/or vehicle). In some embodiments, the individual has an improvement VAS five months or less (e.g., three months) after receiving the keratolytic agent (e.g., selenium sulfide). In some embodiments, the individual has an improvement in average VAS with continued use of the keratolytic agent (e.g., selenium sulfide), such as continued use through 6-months. In some embodiments, the individual has a significant improvement in eye dryness VAS at 6 months of receiving the keratolytic agent (e.g., selenium sulfide). In some embodiments, the individual has a significant improvement in eye dryness VAS within three months or less (e.g., 1.5 months) of receiving the keratolytic agent (e.g., selenium sulfide). In some embodiments, the individual has a significant improvement in itching VAS within five months or less (e.g., three months) of receiving the keratolytic agent (e.g., selenium sulfide).

In some embodiments, In some embodiments, In some embodiments, In some embodiments, In some embodiments, the individual has an improvement in meibomian gland score (MGS) (e.g., compared to baseline and/or vehicle). In some embodiments, the individual has an improvement in MGS from baseline and/or vehicle of ten or more (e.g., twelve or more). In some embodiments, the individual has an improvement in MGS from baseline and/or vehicle five months or less (e.g., three months) after receiving the keratolytic agent (e.g., selenium sulfide). In some embodiments, the individual has an improvement in MGS with continued use of the keratolytic agent (e.g., selenium sulfide), such as continued use through 6-months.

In some embodiments, the individual has an improvement in tear breakup time (TBUT) (e.g., compared to baseline and/or vehicle). In some embodiments, the individual has an improvement in TBUT from baseline and/or vehicle of eight or more (e.g., ten or more). In some embodiments, the individual has an improvement in TBUT from baseline and/or vehicle five months or less (e.g., three months) after receiving the keratolytic agent (e.g., selenium sulfide). In some embodiments, the individual has an improvement in TBUT with continued use of the keratolytic agent (e.g., selenium sulfide), such as continued use through 6-months.

In some embodiments, the individual has an improvement in TBUT and total OSDI (from baseline and/or vehicle) about three months after receiving the keratolytic agent (e.g., selenium sulfide). In some embodiments, the individual has an improvement in TBUT and total OSDI with continued use of the keratolytic agent (e.g., selenium sulfide), such as continued use through 6-months.

Provided in some embodiments herein is a method for identifying a clinically relevant sign or symptom in an individual (or for a disease state), the method comprising using a threshold for measuring a (e.g., physical, such as gland) morphology or morphology threshold.

Provided in some embodiments herein is a method for identifying a clinically relevant sign or symptom in an individual (or for a disease state), the method comprising using a morphology threshold measure (e.g., a physical morphology threshold measure, such as a gland morphology threshold measure (e.g., MGS)).

Provided in some embodiments herein is a method for using a morphology threshold measure (e.g., a physical morphology threshold measure, such as a physical morphology threshold measure (e.g., MGS)) to predict a change in a clinically relevant sign or a symptom in an individual (or for a disease state).

Provided in some embodiments herein is a method for using a morphology threshold measure (e.g., a physical morphology threshold measure, such as a physical morphology threshold measure (e.g., MGS)) to predict a change in another sign (e.g., TBUT) or symptom (e.g., as determined using OSDI) that is clinically relevant in an individual (or for a disease state).

Provided in some embodiments herein is a method for identifying a clinically relevant sign or symptom in an individual (or for a disease state), the method comprising determining and/or using a threshold for measuring a (e.g., physical, such as gland) morphology, such as wherein modification of the morphology (e.g., above or below the threshold) in the individual results in a change in the clinically relevant sign or symptom.

Provided in some embodiments herein is a method for identifying a clinically relevant morphology or morphology threshold for a disease state, the method comprising determining a morphology state that when changed results in a change in a clinically relevant sign or symptom (e.g., associated with the disease state).

Provided in some embodiments herein is a method for treating a disease state, the method comprising (i) evaluating a morphology state (e.g., associated with the disease state), (ii) determining whether the morphology state is above or below a threshold, and (iii) treating the disease state in an individual having an abnormal morphology state (e.g., wherein an abnormal morphology state is above or below the threshold).

In some embodiments, the method comprises treating the disease state comprises administering a therapeutic agent for treating the disease state to the individual.

Provided in some embodiments herein is a method for improving a sign or symptom of a disease state in an individual, the method comprising:
i. scoring a morphological feature (e.g., associated with the disease state) to determine a morphological score;
ii. determining if the morphological score is abnormal relative to a threshold score; and
iii. providing a therapeutic agent (e.g., for the disease state) to the individual (e.g., at least until a normal morphological score is achieved).

In some embodiments, the disease state is an ocular disorder, the morphological feature is open meibomian glands and/or a change in meibum quality, and the threshold score is an MGS of 12 (e.g., wherein >12 is a normal morphological state and ≤12 is an abnormal morphological state).

In some embodiments, the disease state is MGD (or dry eye or blepharitis or demodex (infestation) or contact lens discomfort).

In some embodiments, the method further comprises treating the disorder until a change (e.g., improvement) in an objective measure (e.g., TBUT) or symptom (e.g., OSDI) is achieved.

In some embodiments, the symptom is measured using OSDI, SPEED, or VAS (or a subscale thereof).

Provided in some embodiments herein is a method for treating a first disease state in an individual, the method comprising (i) evaluating a morphological state to determine a morphological score indicative of a second disease state in an individual, (ii) comparing the morphological score to a morphological threshold score, (iii) determining if the morphological score is normal or abnormal, and (iv) treating the second disease state prior to or while treating the first disease state.

In some embodiments, the method further comprises enriching a clinical trial population (e.g. the clinical trial population including individuals with an abnormal (morphological) score).

In some embodiments, the method further comprises treating one or more individual of a clinical trial population (e.g. the clinical trial population including individuals with an abnormal (morphological) score) with a therapeutic agent during or before a clinical trial.

In some embodiments, the therapeutic agent is a meibum enhancer. In some embodiments, the therapeutic agent is a keratolytic agent. In some embodiments, the therapeutic agent is selenium disulfide. Unless stated otherwise herein, the term selenium sulfide and selenium disulfide are used interchangeably herein.

Provided in some embodiments herein is a method for treating a first disease state in an individual, the method comprising (i) evaluating a morphological state to determine a morphological score indicative of a second disease state in an individual, (ii) comparing the morphological score to a morphological threshold score, (iii) determining if the morphological score is normal or abnormal, (iv) enriching a clinical trial population (e.g. the clinical trial population including individuals with an abnormal (morphological) score), and (v) treating one or more individual of the clinical trial population with a therapeutic agent during or before a clinical trial.

In some embodiments, the first disease state is a first ocular disorder and the second disease state is a second ocular disorder (e.g., and the second ocular disorder is MGD).

In some embodiments, the morphological state is open meibomian glands and the threshold score is an MGS of 12.

In some embodiments, the first disease state is a disease associated with a clinical trial and the method is used to enhance a clinical trial population, such as to improve clinical trial outcomes (e.g., a change in a morphology score of a gland that predicts an inability to wear a lens as designed or desired).

In some embodiments, the keratolytic agent is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, gluthatione, dithiothreitol, thiorphan, cysteamine, bucillamine, dimercaprol, 1,1-ethanedithiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamine, prosultiamine, thiram, lipoic acid, lenthionine, ajoene, allicin, gemopatrilat, thioethanol, thiophospholipid, thiocholesterol, 12-mercaptododecanoic acid, 23-(9-mercaptononyl)-3,6,9,12,15,18,21-heptaoxatricosanoic acid, and sulfanegen. In some embodiments, the keratolytic agent is selected from the group consisting of benzoyl peroxide, coal tar, dithranol, salicylic acid, selenium disulfide, alpha-hydroxy acid, urea, lactic acid, sodium thioglycolate, zinc pyrithione, or zinc L-pyrrolidone carboxylate.

In some embodiments, the keratolytic agent is selenium disulfide.

In some embodiments, a combination of keratolytic agents are provided to or around the eye of the individual (e.g., wherein a first keratolytic agent is provided for a first period of time and a second keratolytic agent is provided for a second period of time).

In some embodiments, the keratolytic agent (e.g., selenium sulfide) is administered to or around the eye of the individual at a concentration of about 0.1 wt. % to about 10 wt. %. In some embodiments, the keratolytic agent (e.g., selenium sulfide) is administered to or around the eye of the individual at concentration of about 0.1 wt. % to about 2 wt. % of selenium disulfide.

In some embodiments, the keratolytic agent (e.g., selenium sulfide) is administered to or around the eye of the individual at concentration of about 0.5 wt. % of selenium disulfide.

In some embodiments, the keratolytic agent (e.g., selenium sulfide) is administered to or around the eye of the individual at concentration of about 1 wt. % of selenium disulfide.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts the protocol design of Example 1.

FIG. 2 shows a statistically significant improvement in signs, MGYLS change from baseline, compared to vehicle.

FIG. 3 shows a statistically significant improvement in symptoms, total OSDI change from baseline, compared to vehicle.

FIG. 4 shows a statistically significant improvement in number of open glands (MGYLS change from baseline).

FIG. 5 shows a statistically significant and clinically meaningful improvement in number of open glands. Analysis compared to vehicle.

FIG. 6 shows a statistically significant improvement in quality of meibum. Analysis compared to vehicle.

FIG. 7 shows a statistically significant and clinically meaningful improvement in the quality of meibum. Analysis compared to vehicle.

FIG. 8 shows a statistically significant improvement in tear breakup time (TBUT) (change from baseline).

FIG. 9 shows a statistically significant improvement in individuals with normal TBUT. Analysis compared to vehicle.

FIG. 10 shows a statistically significant percentage of individuals who become asymptomatic on OSDI. Analysis compared to vehicle.

FIG. 11A shows a statistically significant and clinically meaningful improvement in ocular symptoms (change from baseline).

FIG. 11B shows a statistically significant and clinically meaningful improvement in environmental triggers (change from baseline).

FIG. 11C shows a statistically significant and clinically meaningful improvement in visual function (change from baseline).

FIG. 12 shows a statistically significant improvement in SPEED score (change from baseline).

FIG. 13 shows a statistically significant improvement in average VAS (change from baseline).

FIG. 14 shows a statistically significant improvement in eye dryness VAS score (change from baseline).

FIG. 15A shows a statistically significant improvement in pain VAS score (change from baseline).

FIG. 15B shows a statistically significant improvement in photophobia VAS score (change from baseline).

FIG. 15C shows a statistically significant improvement in burning/stinging VAS score (change from baseline).

FIG. 15D shows a statistically significant improvement in itching VAS score (change from baseline).

FIG. 15E shows a statistically significant improvement in foreign body VAS score (change from baseline).

FIG. 15F shows a statistically significant improvement in eye discomfort VAS score (change from baseline).

FIG. 16 shows total OSDI change from baseline for individuals with MGS scores less than or equal to 12 and greater than 12.

FIG. 17 shows total TBUT change from baseline for individuals with MGS scores less than or equal to 12 and greater than 12.

FIG. 18 shows total TBUT change from baseline for individuals with MGYLS scores less than or equal to the mean MGYLS score and greater than the mean MGYLS score.

FIG. 19 shows recruit, randomization, and study flow of Example 1.

FIG. 20 shows a reduction of adverse events when using a composition described herein over time.

### DETAILED DESCRIPTION OF THE INVENTION

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

The terms “treat,” “treating,” or “treatment” as used herein, include reducing, alleviating, abating, ameliorating, managing, relieving, or lessening the symptoms associated with a disease, disease state, condition, or indication (e.g., provided herein) in either a chronic or acute therapeutic scenario. Also, treatment of a disease or disease state described herein includes the disclosure of use of such compound or composition for the treatment of such disease, disease state, disorder, or indication.

The term “keratolytic agent” as used herein refers to an agent that softens, disrupts, dissolves, solubilizes, or loosens a keratinized obstruction, or prevents the formation of a keratinized obstruction. In some instances, keratolytic agents are used to promote softening and dissolution of keratin.

Concentrations of agents provided herein are based on any suitable measurement, such as wt. %, w/w %, or w/v %. In specific instances, the concentration is wt. % (e.g., w/w % or w/v %).

In some embodiments, an individual receiving a composition described herein has a sign outcome for an ocular disorder, such as meibomian gland dysfunction (MGD) and/or dry eye disease (DED). In some embodiments, an individual receiving a composition described herein has a symptom outcome for an ocular disorder, such as MGD and/or DED. In some embodiments, an individual receiving a composition described herein has a sign and symptom outcome for an ocular disorder, such as MGD and/or DED. In some embodiments, a composition described herein provides an improved sign outcome for an ocular disorder, such as for MGD and/or DED. In some embodiments, a composition described herein provides an improved symptom outcome for an ocular disorder, such as for MGD and/or DED. In some embodiments, a composition described herein provides an improved sign and symptom outcome for an ocular disorder, such as for MGD and/or DED.

Provided in some embodiments herein is a method for treating an ocular disorder in an individual, the method comprising providing a keratolytic agent to or around the eye of the individual until an objective measure and symptom of the ocular disorder are improved. In some embodiments, the keratolytic agent is selenium disulfide.

Provided in some embodiments herein is a method for treating an ocular disorder in an individual.

In some embodiments, the method comprises evaluating an objective measure for an ocular indication to determine an initial objective score. In some embodiments, the method comprises providing a keratolytic agent to or around the eye of the individual. In some embodiments, the keratolytic agent is selenium sulfide. In some embodiments, the method comprises evaluating the objective measure to determine a second objective score after a keratolytic agent (e.g., selenium sulfide) has been provided to or around the eye of the individual at least one time. In some embodiments, the method comprises determining if the second objective score is an improvement over the initial objective score. In some embodiments, such as if the second objective score is an improvement over the initial objective score, the method comprises providing a keratolytic agent to or around the eye of the individual.

In some embodiments, the method further comprises evaluating a symptom for the ocular indication to determine an initial symptom score.

In some embodiment, the method further comprises evaluating the symptom or initial symptom score to determine a second symptom score.

In some embodiments, such as if the second symptom score is an improvement over the initial symptom score, the method further comprises providing keratolytic agent (e.g., selenium sulfide) to or around the eye of the individual.

In some embodiments, the method comprises evaluating an objective measure for an ocular indication to determine an initial objective score. In some embodiments, the method comprises evaluating a symptom for the ocular indication to determine an initial symptom score. In some embodiments, the method comprises providing a keratolytic agent to or around the eye of the individual. In some embodiments, the keratolytic agent is selenium sulfide. In some embodiments, the method comprises evaluating the objective measure to determine a second objective score after a keratolytic agent (e.g., selenium sulfide) has been provided to or around the eye of the individual at least one time. In some embodiments, the method comprises determining if the second objective score is an improvement over the initial objective score. In some embodiments, such as if the second objective score is an improvement over the initial objective score, the method comprises providing a keratolytic agent to or around the eye of the individual. In some embodiment, the method comprises evaluating the symptom or initial symptom score to determine a second symptom score. In some embodiments, such as if the second symptom score is an improvement over the initial symptom score, the method comprises providing keratolytic agent (e.g., selenium sulfide) to or around the eye of the individual.

In some embodiments, the initial objective score is determined before providing a keratolytic agent (e.g., selenium sulfide) to or around the eye of the individual. In some embodiments, the initial objective score is a baseline measurement of the objective measure.

In some embodiments, improvement in a symptom or objective measure is determined by comparing a second score (e.g., the second objective score or the second symptom score) to a baseline measurement, such as an initial score. In some embodiments, improvement in objective measure is determined by comparing the second objective score to a baseline measurement, such as the initial objective score. In some embodiments, improvement in symptom is determined by comparing the second symptom score to a baseline measurement, such as the initial symptom score.

In some embodiments, improvement in a symptom or objective measure is determined by comparing a second score (e.g., the second objective score or the second symptom score) to a control (e.g., vehicle or placebo) measurement, such as a second score of the control, which is measured after the initial score. In some embodiments, improvement in objective measure is determined by comparing the second objective score to a control (e.g., vehicle or placebo) measurement, such as a second score of the control, which is measured after the initial score. In some embodiments, improvement in symptom is determined by comparing the second symptom score to a control (e.g., vehicle or placebo) measurement, such as a second score of the control, which is measured after the initial score.

In some embodiments, an improvement in the initial objective score is observed within about 5 months or less after providing the keratolytic agent (e.g., selenium sulfide) to or around the eye of the individual. In some embodiments, a significant improvement in the initial objective score is observed within about 5 months or less after providing the keratolytic agent (e.g., selenium sulfide) to or around the eye of the individual. In some embodiments, a significant improvement in the initial objective score is observed within about 3 months or less after providing the keratolytic agent (e.g., selenium sulfide) to or around the eye of the individual. In some embodiments, a significant improvement in the initial objective score is observed within about 1.5 months or less after providing the keratolytic agent (e.g., selenium sulfide) to or around the eye of the individual. In some embodiments, a significant improvement in the initial objective score is observed within about 14 days or less after providing the keratolytic agent (e.g., selenium sulfide) to or around the eye of the individual.

In some embodiments, an improvement in the initial symptom score is observed within about 5 months or less after providing the keratolytic agent (e.g., selenium sulfide) to or around the eye of the individual. In some embodiments, a significant improvement in the initial symptom score is observed within about 5 months or less after providing the keratolytic agent (e.g., selenium sulfide) to or around the eye of the individual. In some embodiments, a significant improvement in the initial symptom score is observed within about 3 months or less after providing the keratolytic agent (e.g., selenium sulfide) to or around the eye of the individual. In some embodiments, a significant improvement in the initial symptom score is observed within about 1.5 months or less after providing the keratolytic agent (e.g., selenium sulfide) to or around the eye of the individual.

In some embodiments, an improvement in the initial objective score is observed before an improvement in the symptom score. In some embodiments, a significant improvement in the initial objective score is observed before an improvement in the symptom score. In some embodiments, an improvement in the initial objective score is observed before a significant improvement in the symptom score. In some embodiments, a significant improvement in the initial objective score is observed before a significant improvement in the symptom score.

In some embodiments, an improvement in the symptom score is observed several weeks or months after an improvement in the initial objective score is observed. In some embodiments, a significant improvement in the symptom score is observed several weeks or months after an improvement in the initial objective score is observed. In some embodiments, an improvement in the symptom score is observed several weeks or months after a significant improvement in the initial objective score is observed. In some embodiments, a significant improvement in the symptom score is observed several weeks or months after a significant improvement in the initial objective score is observed.

In some embodiments, the symptom or symptom score gets worse for a period of time after providing the keratolytic agent (e.g., selenium sulfide) to or around the eye of the individual. In some embodiments, the symptom or symptom score initially gets worse for a period of time after providing the keratolytic agent (e.g., selenium sulfide) to or around the eye of the individual. In some embodiments, the second symptom score is worse than the first symptom score for a period of time after providing the keratolytic agent (e.g., selenium sulfide) to or around the eye of the individual. In some embodiments, the symptom or symptom score is worse than a baseline measurement for a period of time after providing the keratolytic agent (e.g., selenium sulfide) to or around the eye of the individual. In some embodiments, the symptom or symptom score is worse than a control measurement for a period of time after providing the keratolytic agent (e.g., selenium sulfide) to or around the eye of the individual. In some embodiments, the symptom or symptom score is worse than a baseline or control measurement for at least one week (e.g., two weeks or more, one month or more, or two months or more) after providing the keratolytic agent (e.g., selenium sulfide) to or around the eye of the individual. In some embodiments, the symptom or symptom score is worse than a baseline or control measurement for about 14 days or more after providing the keratolytic agent (e.g., selenium sulfide) to or around the eye of the individual. In some embodiments, the symptom or symptom score is worse than a baseline or control measurement for about 1.5 months or more after providing the keratolytic agent (e.g., selenium sulfide) to or around the eye of the individual. In some embodiments, the symptom or symptom score is worse than a baseline or control measurement for about 3 months or more after providing the keratolytic agent (e.g., selenium sulfide) to or around the eye of the individual.

In some embodiments, a (significant) improvement in the symptom score and a (significant) improvement in the initial objective score is observed at least about 14 days after providing the keratolytic agent (e.g., selenium sulfide) to or around the eye of the individual. In some embodiments, a (significant) improvement in the symptom score and a (significant) improvement in the initial objective score is observed at least about one month after providing the keratolytic agent (e.g., selenium sulfide) to or around the eye of the individual. In some embodiments, a (significant) improvement in the symptom score and a (significant) improvement in the initial objective score is observed at least about two months after providing the keratolytic agent (e.g., selenium sulfide) to or around the eye of the individual. In some embodiments, an improvement in the symptom score and an improvement in the initial objective score is observed about 3 months after providing the keratolytic agent (e.g., selenium sulfide) to or around the eye of the individual. In some embodiments, a significant improvement in the symptom score and an improvement in the initial objective score is observed about 3 months after providing the keratolytic agent (e.g., selenium sulfide) to or around the eye of the individual. In some embodiments, an improvement in the symptom score and a significant improvement in the initial objective score is observed about 3 months after providing the keratolytic agent (e.g., selenium sulfide) to or around the eye of the individual. In some embodiments, a significant improvement in the symptom score and a significant improvement in the initial objective score is observed about 3 months after providing the keratolytic agent (e.g., selenium sulfide) to or around the eye of the individual.

In some embodiments, the method further comprises comparing the improvement of an objective score to an improvement of a symptom score, such as discussed hereinbelow. In some embodiments, improvement of the symptom score is determined after improvement of the objective score. In some embodiments, improvement of the symptom score is determined several weeks after improvement of the objective score. In some embodiments, improvement of the objective score is determined at least about 7 days or more after providing the keratolytic agent (e.g., selenium sulfide) to or around the eye of the individual. In some embodiments, improvement of the objective score is determined about 14 days after providing the keratolytic agent (e.g., selenium sulfide) to or around the eye of the individual. In some embodiments, improvement of the objective score is determined about 1.5 months after providing the keratolytic agent (e.g., selenium sulfide) to or around the eye of the individual. In some embodiments, improvement of the objective score is determined about 3 months after providing the keratolytic agent (e.g., selenium sulfide) to or around the eye of the individual. In some embodiments, improvement of the symptom score is determined at least about 7 days or more after providing the keratolytic agent (e.g., selenium sulfide) to or around the eye of the individual. In some embodiments, improvement of the symptom score is determined about 14 days after providing the keratolytic agent (e.g., selenium sulfide) to or around the eye of the individual. In some embodiments, improvement of the symptom score is determined about 1.5 months after providing the keratolytic agent (e.g., selenium sulfide) to or around the eye of the individual. In some embodiments, improvement of the symptom score is determined about 3 months after providing the keratolytic agent (e.g., selenium sulfide) to or around the eye of the individual. In some embodiments, improvement of the objective score is determined about 14 days after providing the keratolytic agent (e.g., selenium sulfide) to or around the eye of the individual and the symptom score is determined about 1 to 3 months after providing the keratolytic agent (e.g., selenium sulfide) to or around the eye of the individual. In some embodiments, improvement of the objective score is determined about 1 to 3 months after providing the keratolytic agent (e.g., selenium sulfide) to or around the eye of the individual and the symptom score is determined about 1 to 3 months after providing the keratolytic agent (e.g., selenium sulfide) to or around the eye of the individual. In some embodiments, improvement of the objective score and the symptom score is determined about 3 months after providing the keratolytic agent (e.g., selenium sulfide) to or around the eye of the individual.

In some embodiments, the second objective score is compared to a first objective score. In some embodiments, the first objective score is the initial objective score. In some embodiments, the first objective score is determined after the initial objective score is determined. In some embodiments, the second objective score is determined after providing the keratolytic agent (e.g., selenium sulfide) to or around the eye of the individual. In some embodiments, the second objective score is compared to a first objective score and determined after providing the keratolytic agent (e.g., selenium sulfide) to or around the eye of the individual.

In some embodiments, the second symptom score is compared to a first symptom score. In some embodiments, the first symptom score is the initial symptom score. In some embodiments, the first symptom score is determined after the initial symptom score is determined. In some embodiments, the second symptom score is determined after providing the keratolytic agent (e.g., selenium sulfide) to or around the eye of the individual. In some embodiments, the second symptom score is compared to a first symptom score and determined after providing the keratolytic agent (e.g., selenium sulfide) to or around the eye of the individual.

In some embodiments, the second objective score is an improvement over the initial objective score. In some embodiments, an improvement in the objective measure is determined after providing the keratolytic agent (e.g., selenium sulfide) to or around the eye of the individual. In some embodiments, the second objective score is an improvement over the initial objective score and the improvement is determined after providing the keratolytic agent (e.g., selenium sulfide) to or around the eye of the individual.

In some embodiments, the second symptom score is an improvement over the initial symptom score. In some embodiments, an improvement in the symptom measure is determined after providing the keratolytic agent (e.g., selenium sulfide) to or around the eye of the individual. In some embodiments, the second symptom score is an improvement over the initial symptom score and the improvement is determined after providing the keratolytic agent (e.g., selenium sulfide) to or around the eye of the individual.

In some embodiments, the second objective score is not an improvement over the initial objective score. In some embodiments, the second objective score is determined after providing the keratolytic agent (e.g., selenium sulfide) to or around the eye of the individual. In some embodiments, the second objective score is not an improvement over the initial objective score and the second objective score is determined after providing the keratolytic agent (e.g., selenium sulfide) to or around the eye of the individual. In some embodiments, such as if the second objective score is not an improvement over the initial objective score, the method further comprises providing keratolytic agent (e.g., selenium sulfide) to or around the eye of the individual.

In some embodiments, the method comprises providing keratolytic agent (e.g., selenium sulfide) to or around the eye of the individual until there is an improvement in the objective measure (e.g., sign). In some embodiments, the method comprises providing keratolytic agent (e.g., selenium sulfide) to or around the eye of the individual until the second objective score is an improvement over an objective score, such as the initial objective score.

In some embodiments, the method comprises providing keratolytic agent (e.g., selenium sulfide) to or around the eye of the individual until there is an improvement in the symptom. In some embodiments, the method comprises providing keratolytic agent (e.g., selenium sulfide) to or around the eye of the individual until the second symptom score is an improvement over a symptom score, such as the initial symptom score.

In some embodiments, such as if the second symptom score is not an improvement over the initial symptom score, the method further comprises providing keratolytic agent (e.g., selenium sulfide) to or around the eye of the individual. In some embodiments, such as if the second symptom score is not an improvement over the initial symptom score, the method further comprises providing keratolytic agent (e.g., selenium sulfide) to or around the eye of the individual. until the second symptom score is an improvement over the initial symptom score.

In some embodiments, the keratolytic agent (e.g., selenium sulfide) is provided to or around the eye of the individual at least until determining that the second objective score is an improvement over the initial objective score. In some embodiments, the keratolytic agent (e.g., selenium sulfide) is provided to or around the eye of the individual at least until determining that that the second symptom score is an improvement over the initial symptom score. In some embodiments, the keratolytic agent (e.g., selenium sulfide) is provided to or around the eye of the individual at least until determining that the second objective score is an improvement over the initial objective score and that the second symptom score is an improvement over the initial symptom score.

In some embodiments, the method further comprises providing the keratolytic agent (e.g., selenium sulfide) to or around the eye of the individual is maintained after observing that the second objective score is an improvement over the initial objective score. In some embodiments, the method further comprises providing the keratolytic agent (e.g., selenium sulfide) to or around the eye of the individual is maintained after observing that the second symptom score is an improvement over the initial symptom score. In some embodiments, the method further comprises providing the keratolytic agent (e.g., selenium sulfide) to or around the eye of the individual is maintained after observing that the second objective score is an improvement over the initial objective score and that the second symptom score is an improvement over the initial symptom score.

In some embodiments, the objective measure is a morphology or a biomarker.

In some embodiments, the objective measure is of a morphology or a biomarker. In some embodiments, the morphology is of a meibomian gland. In some embodiments, the morphology is a change in the morphology of a meibomian gland.

In some embodiments, an improvement in opening meibomian glands of the individual is observed after providing the keratolytic agent (e.g., selenium sulfide) to or around the eye of the individual. In some embodiments, a significant improvement in opening meibomian glands of the individual is observed after providing the keratolytic agent (e.g., selenium sulfide) to or around the eye of the individual.

Provided in some embodiments herein is a method for opening meibomian glands in an individual. In some embodiments, the meibomian glands are obstructed. In some embodiments, the meibomian glands are obstructed with keratinized material. In some embodiments, the method comprises evaluating an objective measure for an ocular indication to determine an initial objective score. In some embodiments, the objective measure is meibomian glands yielding liquid secretion score (MGYLS). In some embodiments, the method comprises providing a keratolytic agent (e.g., selenium sulfide) to or around the eye of the individual. In some embodiments, the method comprises evaluating the objective measure to determine a second objective score after keratolytic agent (e.g., selenium sulfide) has been provided to or around the eye of the individual at least one time. In some embodiments, the method comprises determining if the second objective score is an improvement over the initial objective score. In some embodiments, such as if the second objective score is an improvement over the initial objective score, the method comprises providing keratolytic agent (e.g., selenium sulfide) to or around the eye of the individual.

Provided in some embodiments herein is a method for improving tear stability in an individual. In some embodiments, the method comprises evaluating an objective measure for an ocular indication to determine an initial objective score. In some embodiments, the objective measure is tear break up time (TBUT). In some embodiments, the method comprises providing a keratolytic agent (e.g., selenium sulfide) to or around the eye of the individual. In some embodiments, the method comprises evaluating the objective measure to determine a second objective score after keratolytic agent (e.g., selenium sulfide) has been provided to or around the eye of the individual at least one time. In some embodiments, the method comprises determining if the second objective score is an improvement over the initial objective score. In some embodiments, such as if the second objective score is an improvement over the initial objective score, the method comprises providing keratolytic agent (e.g., selenium sulfide) to or around the eye of the individual.

In some embodiments, an improvement in meibum quality of the individual is observed after providing the keratolytic agent (e.g., selenium sulfide) to or around the eye of the individual. In some embodiments, an improvement in meibum quantity of the individual is observed after providing the keratolytic agent (e.g., selenium sulfide) to or around the eye of the individual. In some embodiments, an improvement in meibum quality and quantity of the individual is observed after providing the keratolytic agent (e.g., selenium sulfide) to or around the eye of the individual. In some embodiments, the improvement in meibum quality and/or quantity is a significant improvement.

In some embodiments, a (significant) improvement in opening meibomian glands, meibum quality, and/or meibum quantity of the individual is observed within about 5 months or less (e.g., 3 months or less or 1.5 months or less) after providing the keratolytic agent (e.g., selenium sulfide) to or around the eye of the individual. In some embodiments, the individual has an improvement in opening meibomian glands, meibum quality, and/or meibum quantity with continued use of the keratolytic agent (e.g., selenium sulfide), such as continued use through 6-months.

In some embodiments, the morphology or biomarker is clogged and/or blocked pores, closed meibomian glands, meibum color, meibum quality, meibum quantity, tear open time, lid swelling, lid hyperemia, or conjunctival hyperemia.

In some embodiments, the morphology is clogged and/or blocked pores, closed meibomian glands, meibum color, meibum quality, meibum quantity, or tear open time. In some embodiments, the morphology (or change thereof) is determined with visual inspection, staining, or imaging. In some embodiments, the visual inspection is via a microscope). In some embodiments, the visual inspection is via a slit-lamp microscope). In some embodiments, staining is ocular staining. In some embodiments, imaging is via infra-red imaging. In some embodiments, imaging is via meibography.

In some embodiments, the biomarker is a molecular biomarker, a physiological biomarker, a radiographic biomarker, a histologic biomarker, or a measurement thereof. In some embodiments, the molecular biomarker is measured within a biological sample (e.g., serum, plasma, biopsy, meibum, or tear fluid). In some embodiments, the physiological biomarker is a measurement of a function or process of a system (e.g., an eye, eyelid, or a gland or function thereof). In some embodiments, the radiographic biomarker is measured with imaging. In some embodiments, the histologic biomarker is measured by molecular or biochemical alterations within fluids, cells, or tissues.

In some embodiments, the biomarker is a measure of ocular (surface) function or symptoms thereof. In some embodiments, a symptom is a self-reported symptom. In some embodiments, the biomarker is a measure of meibomian gland function. In some embodiments, the biomarker is observed in meibum. In some embodiments, the biomarker is a molecule observed in meibum. In some embodiments, the biomarker is selected from the group consisting of an interleukin, an amino acid, a cadherin, an eicosanoid, a carbohydrate, and proteins. In some embodiments, the biomarker is altered in meibomian gland dysfunction (MGD) compared to a control. In some embodiments, the amount of the biomarker is altered in MGD compared to a control. In some embodiments, the molecular biomarker is a antileukoproteinase, phospholipase A2, and lactoperoxidase. In some instances, there is a difference in the concentration of a biomarker in tears of an individual with MGD and in tears of an individual with dry eye disease.

In some embodiments, the biomarker is meibomian glands yielding liquid secretion score (MGYLS), total ocular surface disease index (OSDI) score, standard patient evaluation of eye dryness (SPEED), meibomian gland score (MGS), or tear breakup time (TBUT).

In some embodiments, the symptom score is visual analogue scale (VAS) (e.g., pain VAS, photophobia VAS, burning/stinging VAS, itching VAS, foreign body VAS, or eye discomfort VAS), SPEED, or contact lens dry eye questionnaire-8 (CLDQ8). In some embodiments, the symptom score is determined by an average VAS, SPEED, or CLDQ8. In some embodiments, VAS is a measurement of pain (e.g., pain VAS), photophobia (e.g., photophobia VAS), burning or stinging (e.g., burning/stinging VAS), itching (e.g., itching VAS), foreign body (e.g., foreign body VAS), or eye discomfort (e.g., eye discomfort VAS).

Provided in some embodiments herein is a method for improving a symptom in or around an eye of an individual. In some embodiments, the method comprises evaluating an objective measure for an ocular indication to determine an initial objective score. In some embodiments, the method comprises evaluating the symptom for the ocular indication to determine an initial symptom score. In some embodiments, the initial symptom score is measured by ocular surface disease index (OSDI). In some embodiments, the initial symptom score is measured by visual analog scale (VAS). In some embodiments, the initial symptom score is measured by total OSDI and average VAS. In some embodiments, the method comprises providing a keratolytic agent (e.g., selenium sulfide) to or around the eye of the individual. In some embodiments, the method comprises evaluating the objective measure to determine a second objective score after keratolytic agent (e.g., selenium sulfide) has been provided to or around the eye of the individual at least one time. In some embodiments, the method comprises determining if the second objective score is an improvement over the initial objective score. In some embodiments, such as if the second objective score is an improvement over the initial objective score, the method comprises providing keratolytic agent (e.g., selenium sulfide) to or around the eye of the individual. In some embodiments, the method comprises evaluating the symptom or initial symptom score to determine a second symptom score. In some embodiments, such as if the second symptom score is an improvement over the initial symptom score, the method comprises providing keratolytic agent (e.g., selenium sulfide) to or around the eye of the individual.

Provided in some embodiments herein is a method for treating an ocular disorder in an individual. In some embodiments, the method comprises evaluating a measure of TBUT to determine an initial objective score. In some embodiments, the method comprises evaluating a measure of total OSDI to determine an initial symptom score. In some embodiments, the method comprises providing a keratolytic agent (e.g., selenium sulfide) to or around the eye of the individual. In some embodiments, the method comprises evaluating the measure of TBUT to determine a second objective score after keratolytic agent (e.g., selenium sulfide) has been provided to or around the eye of the individual at least one time. In some embodiments, the method comprises determining if the second objective score is an improvement over the initial objective score. In some embodiments, such as if the second objective score is an improvement over the initial objective score, the method comprises providing keratolytic agent (e.g., selenium sulfide) to or around the eye of the individual. In some embodiments, the method comprises evaluating the measure of total OSDI or initial symptom score to determine a second symptom score. In some embodiments, such as if the second symptom score is an improvement over the initial symptom score, the method comprises providing keratolytic agent (e.g., selenium sulfide) to or around the eye of the individual.

In some embodiments, the symptom of the ocular indication is eye dryness, pain, photophobia, burning, stinging, itching, grittiness, or eye discomfort. the symptom being evaluated in the individual is eye dryness, pain, photophobia, burning, stinging, itching, grittiness, or eye discomfort. In some instances, an individual reporting grittiness is feeling like a foreign body is in the eye.

In some embodiments, the symptom of the ocular indication (being evaluated) is performance of daily activities, such as night driving or eye fatigue. In some instances, the symptom of the ocular indication (being evaluated) is heavy lids, eye dryness, pain, photophobia, burning, stinging, itching, grittiness (e.g., feeling like a foreign body is in the eye), or eye discomfort.

Provided in some embodiments herein is a method for treating an ocular disorder in an individual. In some embodiments, the method comprises providing a keratolytic agent (e.g., selenium sulfide) to or around the eye of the individual for a first period of time, over which first period of time improvement in an objective measure of the ocular disorder is achieved. In some embodiments, the first period of time is at least one week (e.g., one week or more, two weeks or more, three weeks or more, or four weeks or more). In some embodiments, the first period of time is one to four weeks. In some embodiments, the method comprises providing a keratolytic agent (e.g., selenium sulfide) to or around the eye of the individual for a second period of time, over which second period of time improvement of a symptom of the ocular disorder is achieved. In some embodiments, the second period of time is at least one week (e.g., one week or more, two weeks or more, four weeks or more, eight weeks or more, sixteen weeks or more, or thirty-two weeks or more). In some embodiments, the second period of time is about two to twenty-six weeks. In some embodiments, the method comprises providing a keratolytic agent (e.g., selenium sulfide) to or around the eye of the individual, after which time (e.g., 1 month or more), improvement in the objective measure and/or symptom is maintained.

In some embodiments, the objective measure (of the ocular disorder) remains the same or gets worse, such as during the first period. In some embodiments, the objective measure (of the ocular disorder) remains the same or gets worse, such as during a portion of the second period. In some embodiments, the symptom (of the ocular disorder) remains the same or gets worse, such as during the first period. In some embodiments, the symptom (of the ocular disorder) remains the same or gets worse, such as during a portion of the second period. In some embodiments, the objective measure (of the ocular disorder) and/or the symptom (of the ocular disorder) remains the same or gets worse, such as during a portion of the first period or the second period.

In some embodiments, the objective measure (of the ocular disorder) significantly improves, such as during the first period. In some embodiments, the objective measure (of the ocular disorder) significantly improves, such as during a portion of the second period. In some embodiments, the symptom (of the ocular disorder) significantly improves, such as during the first period. In some embodiments, the symptom (of the ocular disorder) significantly improves, such as during a portion of the second period. In some embodiments, the objective measure (of the ocular disorder) and/or the symptom (of the ocular disorder) significantly improves, such as during a portion of the first period or the second period.

In some embodiments, the keratolytic agent (e.g., selenium sulfide) is administered to the individual monthly, bi-weekly, or once-weekly. In some embodiments, the keratolytic agent (e.g., selenium sulfide) is administered to the individual once-weekly. In some embodiments, Composition 1 is administered to the individual once-weekly. In some embodiments, Composition 2 is administered to the individual once-weekly.

In some embodiments, the keratolytic agent (e.g., selenium sulfide) is administered to the individual for at least one month. In some embodiments, the keratolytic agent (e.g., selenium sulfide) is administered to the individual once-weekly for at least one month. In some embodiments, the keratolytic agent (e.g., selenium sulfide) is administered to the individual for one month or more, two months or more, three months or more, five months or more, or six months or more. In some embodiments, the keratolytic agent (e.g., selenium sulfide) is administered to the individual once-weekly for one month or more, two months or more, three months or more, five months or more, or six months or more.

In some embodiments, an individual described herein has an improvement in lipid secretion.

In some embodiments, the individual has an improvement in meibomian glands yielding liquid secretion score (MGYLS). In some embodiments, the individual has an improvement in MGYLS of three or more, four or more, or five or more. In some embodiments, the individual has an improvement in MGYLS of five or more. In some embodiments, the individual has an improvement in MGYLS five months or less after receiving the keratolytic agent (e.g., selenium sulfide). In some embodiments, the individual has an improvement in MGYLS with continued use of the keratolytic agent (e.g., selenium sulfide) through 6-months. In some embodiments, the individual has an improvement in MGYLS three months or less after receiving the keratolytic agent (e.g., selenium sulfide). In some embodiments, the individual has an improvement in MGYLS two months or less after receiving the keratolytic agent (e.g., selenium sulfide). In some embodiments, the individual has an improvement in MGYLS one month or less after receiving the keratolytic agent (e.g., selenium sulfide). In some embodiments, the individual has an improvement in MGYLS two weeks or less after receiving the keratolytic agent (e.g., selenium sulfide). In some embodiments, the improvement in MGYLS is compared to a baseline measurement. In some embodiments, the improvement in MGYLS is compared to vehicle. In some embodiments, the improvement in MGYLS is compared to a baseline measurement and vehicle.

In some embodiments, the individual has an improvement in meibomian gland score (MGS). In some embodiments, the individual has an improvement in MGS of ten or more, eleven or more, or twelve or more. In some embodiments, the individual has an improvement in MGS of twelve or more. In some embodiments, the individual has an improvement in MGS five months or less after receiving the keratolytic agent (e.g., selenium sulfide). In some embodiments, the individual has an improvement in MGS with continued use of the keratolytic agent (e.g., selenium sulfide), such as continued use through 6-months. In some embodiments, the individual has an improvement in MGS three months or less after receiving the keratolytic agent (e.g., selenium sulfide). In some embodiments, the individual has an improvement in MGS two months or less after receiving the keratolytic agent (e.g., selenium sulfide). In some embodiments, the individual has an improvement in MGS one month or less after receiving the keratolytic agent (e.g., selenium sulfide). In some embodiments, the individual has an improvement in MGS two weeks or less after receiving the keratolytic agent (e.g., selenium sulfide). In some embodiments, the improvement in MGS is compared to a baseline measurement. In some embodiments, the improvement in MGS is compared to vehicle. In some embodiments, the improvement in MGS is compared to a baseline measurement and vehicle.

In some embodiments, the individual has an improvement in tear breakup time (TBUT). In some embodiments, the individual has an improvement in TBUT of eight or more, nine or more, or ten or more. In some embodiments, the individual has an improvement in TBUT of ten or more. In some embodiments, the individual has an improvement in TBUT five months or less after receiving the keratolytic agent (e.g., selenium sulfide). In some embodiments, the individual has an improvement in TBUT with continued use of the keratolytic agent (e.g., selenium sulfide), such as continued use through 6-months. In some embodiments, the individual has an improvement in TBUT three months or less after receiving the keratolytic agent (e.g., selenium sulfide). In some embodiments, the individual has an improvement in TBUT two months or less after receiving the keratolytic agent (e.g., selenium sulfide). In some embodiments, the individual has an improvement in TBUT one month or less after receiving the keratolytic agent (e.g., selenium sulfide). In some embodiments, the individual has an improvement in TBUT two weeks or less after receiving the keratolytic agent (e.g., selenium sulfide). In some embodiments, the improvement in TBUT is compared to a baseline measurement. In some embodiments, the improvement in TBUT is compared to vehicle. In some embodiments, the improvement in TBUT is compared to a baseline measurement and vehicle.

In some embodiments, the individual has an improvement in total ocular surface disease index (OSDI) score. In some embodiments, the individual has an improvement in total OSDI of fifteen or less, fourteen or less, or thirteen or less. In some embodiments, the individual has an improvement in total OSDI of thirteen or less. In some embodiments, the individual has an improvement in total OSDI five months or less after receiving the keratolytic agent (e.g., selenium sulfide). In some embodiments, the individual has an improvement in total OSDI with continued use of the keratolytic agent (e.g., selenium sulfide) through 6-months. In some embodiments, the individual has an improvement in total OSDI three months or less after receiving the keratolytic agent (e.g., selenium sulfide). In some embodiments, the individual has an improvement in total OSDI two months or less after receiving the keratolytic agent (e.g., selenium sulfide). In some embodiments, the individual has an improvement in total OSDI one month or less after receiving the keratolytic agent (e.g., selenium sulfide). In some embodiments, the improvement in total OSDI is compared to a baseline measurement. In some embodiments, the improvement in total OSDI is compared to vehicle. In some embodiments, the improvement in total OSDI is compared to a baseline measurement and vehicle.

In some embodiments, the individual has an improvement in TBUT and total OSDI (from baseline and/or vehicle) about three months after receiving the keratolytic agent (e.g., selenium sulfide).

In some embodiments, a composition described herein (e.g., Composition 1 and/or Composition 2) improves TBUT and OSDI scores, such as over and/or within a period of about three to six months.

In some embodiments, the individual has an improvement in standard patient evaluation of eye dryness (SPEED). In some embodiments, the individual has an improvement in SPEED five months or less after receiving the keratolytic agent (e.g., selenium sulfide). In some embodiments, the individual has an improvement in SPEED with continued use of the keratolytic agent (e.g., selenium sulfide) through 6-months. In some embodiments, the individual has an improvement in SPEED three months or less after receiving the keratolytic agent (e.g., selenium sulfide). In some embodiments, the individual has an improvement in SPEED two months or less after receiving the keratolytic agent (e.g., selenium sulfide). In some embodiments, the Individual has an improvement in SPEED one month or less after receiving the keratolytic agent (e.g., selenium sulfide). In some embodiments, the improvement in SPEED is compared to a baseline measurement. In some embodiments, the improvement in SPEED is compared to vehicle. In some embodiments, the improvement in SPEED is compared to a baseline measurement and vehicle. In some instances, a SPEED score ranges from 0 to 28. In some instances, a SPEED score of 0-4 is classified as mild. In some instances, a SPEED score of 5-7 is classified as moderate. In some instances, a SPEED score of greater than or equal to 8 is classified as severe.

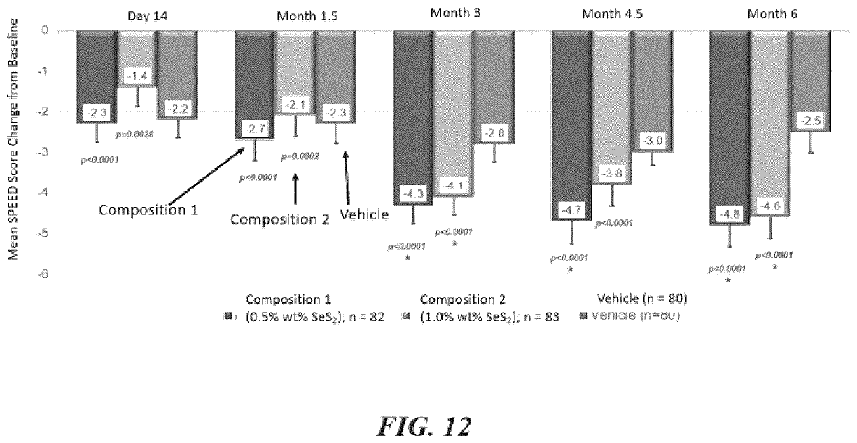

In some instances, a composition provided herein (e.g., Composition 1 and/or Composition 2) provides greater improvement in SPEED scores in comparison to vehicle, such as after 3 months, 4.5 months, and/or 6 months of treatment, see FIG. 12. In some embodiments, a composition described herein (e.g., Composition 1 and/or Composition 2) provides an improvement in SPEED score from baseline to month 3 of treatment of at least 3.5 (e.g., at least 3.75, at least 4, at least 4.5). In some embodiments, a composition described herein (e.g., Composition 1 and/or Composition 2) provides a change in SPEED score from baseline to month 3 of treatment of at most 5 (e.g., at most 4.5, at most 4, at most 3.5). In some embodiments, a composition described herein (e.g., Composition 1 and/or Composition 2) provides a change in SPEED score from baseline to month 3 of treatment of about 4.2. In some embodiments, a composition described herein (e.g., Composition 1 and/or Composition 2) provides a change in SPEED score from baseline to month 4.5 of treatment of about 4.7. In some embodiments, a composition described herein (e.g., Composition 1 and/or Composition 2) provides a change in SPEED score from baseline to month 6 of treatment of about 4.8.

In some embodiments, a composition described herein (e.g., Composition 1 and/or Composition 2) improves MGYLS and OSDI scores, such as over and/or within a period of about three to six months. In some instances, a composition provided herein (e.g., Composition 1 and/or Composition 2) demonstrate statistical benefit over vehicle treatment. In some instances, a composition provided herein (e.g., Composition 1 and/or Composition 2) demonstrate a non-linear dose response (e.g., where a lower effect is observed at higher concentrations), such as when examining sebum production in sebaceous cells.

In some embodiments, a composition described herein (e.g., Composition 1 and/or Composition 2) is safe and well tolerated. In some instances, tolerability to treatment with a composition described herein (e.g., Composition 1 and/or Composition 2) is improved with bedtime dosing, such as twice weekly bedtime dosing.

In some embodiments, the individual has an improvement in average visual analogue scale (VAS). In some embodiments, the individual has an improvement in VAS five months or less after receiving the keratolytic agent (e.g., selenium sulfide). In some embodiments, the individual has an improvement in average VAS with continued use of the keratolytic agent (e.g., selenium sulfide), such as continued use through 6-months. In some embodiments, the individual has an improvement in VAS three months or less after receiving the keratolytic agent (e.g., selenium sulfide). In some embodiments, the individual has an improvement in VAS two months or less after receiving the keratolytic agent (e.g., selenium sulfide). In some embodiments, the individual has an improvement in VAS one month or less after receiving the keratolytic agent (e.g., selenium sulfide). In some embodiments, the improvement in V A S is compared to a baseline measurement. In some embodiments, the improvement in VAS is compared to vehicle. In some embodiments, the improvement in VAS is compared to a baseline measurement and vehicle.

In some embodiments, the individual has an improvement (e.g., a significant improvement) in eye discomfort VAS within six months or less of receiving the keratolytic agent (e.g., selenium sulfide). In some embodiments, the individual has an improvement (e.g., a significant improvement) in eye discomfort VAS at month 6 of receiving the keratolytic agent (e.g., selenium sulfide). In some embodiments, the individual has an improvement (e.g., a significant improvement) in eye discomfort VAS with continued use of the keratolytic agent (e.g., selenium sulfide), such as continued use through 6-months.

In some embodiments, the individual has a significant improvement in eye dryness VAS within three months or less of receiving the keratolytic agent (e.g., selenium sulfide). In some embodiments, the individual has a significant improvement in eye dryness VAS within 1.5 months of receiving the keratolytic agent (e.g., selenium sulfide). In some embodiments, the individual has an improvement in eye dryness VAS with continued use of the keratolytic agent (e.g., selenium sulfide), such as continued use through 6-months.

In some embodiments, the individual has a significant improvement in itching VAS within five months or less of receiving the keratolytic agent (e.g., selenium sulfide). In some embodiments, the individual has a significant improvement in itching VAS within three months of receiving the keratolytic agent (e.g., selenium sulfide). In some embodiments, the individual has an improvement in itching VAS with continued use of the keratolytic agent (e.g., selenium sulfide), such as continued use through 6-months.

In some embodiments, a study described herein (see Example 1) shows that co-primary (e.g., sign+symptom) endpoints met statistical significance and clinically meaningful for a composition described herein (e.g., Composition 1) over vehicle, such as in a single study in ITT population. In some embodiments, such as with use through 6-months, further improvement in (e.g., all) signs and symptoms is measured. In some embodiments, the co-primary (e.g., sign+symptom) endpoints are meibomian glands yielding liquid secretion (MGYLS) (which can be a measure of the number of open glands) and total ocular surface disease index (OSDI) (which can be a measure of the symptoms of ocular irritation in dry eye disease and how they affect functioning related to vision). In some embodiments, there are no meaningful differences between ITT, mITT and PP populations.

In some embodiments, the ocular disorder is selected from the group consisting of meibomian gland dysfunction (MGD), blepharitis, seborrheic blepharitis, Demodex infestation, dry eye syndrome, hyperkeratosis, dermatitis, keratitis, contact lens discomfort, lid wiper epitheliopathy (LWE), Keratoconjunctivitis Sicca, Sjogren's Syndrome, and ocular rosacea.

In some embodiments, the ocular disorder is ocular itching.

In some embodiments, the ocular disorder is meibomian gland dysfunction (MGD).

In some embodiments, the ocular disorder is dye eye.

Provided in some embodiments herein is a method for treating ocular itching in an individual. In some embodiments, the method comprises evaluating an objective measure for an ocular indication to determine an initial objective score. In some embodiments, In some embodiments, the method comprises providing a keratolytic agent (e.g., selenium sulfide) to or around the eye of the individual. In some embodiments, the method comprises evaluating the objective measure to determine a second objective score after keratolytic agent (e.g., selenium sulfide) has been provided to or around the eye of the individual at least one time. In some embodiments, the method comprises determining if the second objective score is an improvement over the initial objective score. In some embodiments, the method comprises if the second objective score is an improvement over the initial objective score, providing keratolytic agent (e.g., selenium sulfide) to or around the eye of the individual.

Provided in some embodiments herein is a method for treating dry eye in an individual. In some embodiments, the method comprises evaluating an objective measure for an ocular indication to determine an initial objective score. In some embodiments, the method comprises providing a keratolytic agent (e.g., selenium sulfide) to or around the eye of the individual. In some embodiments, the method comprises evaluating the objective measure to determine a second objective score after keratolytic agent (e.g., selenium sulfide) has been provided to or around the eye of the individual at least one time. In some embodiments, the method comprises determining if the second objective score is an improvement over the initial objective score. In some embodiments, the method comprises if the second objective score is an improvement over the initial objective score, providing keratolytic agent (e.g., selenium sulfide) to or around the eye of the individual.

In some embodiments, an individual described herein has itchy eyes.

In some embodiments, an individual described herein has MGD.

In some embodiments, an individual described herein has itchy eyes and MGD.

Provided in some embodiments herein is a method for identifying a clinically relevant sign or symptom in an individual. In some embodiments, the method comprises using a threshold for measuring a morphology. In some embodiments, the morphology is a physical morphology. In some embodiments, the morphology is a gland morphology. In some embodiments, the morphology is a meibomian gland morphology. In some embodiments, the method comprises using a threshold for measuring a morphology threshold.

Provided in some embodiments herein is a method for identifying a clinically relevant sign or symptom for a disease state. In some embodiments, the method comprises using a threshold for measuring a morphology. In some embodiments, the morphology is a physical morphology. In some embodiments, the morphology is a gland morphology. In some embodiments, the morphology is a meibomian gland morphology. In some embodiments, the method comprises using a threshold for measuring a morphology threshold.

Provided in some embodiments herein is a method for identifying a clinically relevant sign or symptom in an individual (or for a disease state), the method comprising using a threshold for measuring a (e.g., physical, such as gland) morphology or morphology threshold.

Provided in some embodiments herein is a method for identifying a clinically relevant sign or symptom in an individual or for a disease state. In some embodiments, the method comprises using a morphology threshold measure. In some embodiments, the morphology threshold measure is a physical morphology threshold measure. In some embodiments, the physical morphology threshold measure is MGS.

Provided in some embodiments herein is a method for using a morphology threshold measure to predict a change in a clinically relevant sign or a symptom in an individual or for a disease state. In some embodiments, the morphology threshold measure is a physical morphology threshold measure. In some embodiments, the physical morphology threshold measure is MGS.

Provided in some embodiments herein is a method for using a morphology threshold measure to predict a change in another sign that is clinically relevant in an individual or for a disease state. In some embodiments, the morphology threshold measure is a physical morphology threshold measure. In some embodiments, the physical morphology threshold measure is MGS. In some embodiments, the other sign is TBUT.

Provided in some embodiments herein is a method for using a morphology threshold measure to predict a change in a symptom that is clinically relevant in an individual or for a disease state. In some embodiments, the morphology threshold measure is a physical morphology threshold measure. In some embodiments, the physical morphology threshold measure is MGS. In some embodiments, the symptom is determined using OSDI, VAS, SPEED, or the like. In some embodiments, the symptom is ocular itching. In some embodiments, the symptom is pain.

Provided in some embodiments herein is a method for identifying a clinically relevant sign or symptom in an individual or for a disease state. In some embodiments, the method comprises determining a threshold for measuring a morphology. In some embodiments, the method comprises using a threshold for measuring a morphology. In some embodiments, the morphology is a physical morphology. In some embodiments, the morphology is a gland morphology. In some embodiments, the morphology is a meibomian gland morphology. In some embodiments, modification of the morphology in the individual results in a change in the clinically relevant sign or symptom. In some embodiments, modification of the morphology is above the threshold. In some embodiment, the threshold is an MGS of 12. In some embodiments, modification of the morphology is below the threshold.

Provided in some embodiments herein is a method for identifying a clinically relevant sign or symptom in an individual (or for a disease state), the method comprising determining a threshold for measuring a (e.g., physical, such as gland) morphology, such as wherein modification of the morphology (e.g., above or below the threshold) in the individual results in a change in the clinically relevant sign or symptom.

Provided in some embodiments herein is a method for identifying a clinically relevant morphology for a disease state. In some embodiments, the method comprises determining a morphology state that when changed results in a change in a clinically relevant sign or symptom. In some embodiments, the clinically relevant sign or symptom is associated with the disease state.

Provided in some embodiments herein is a method for identifying a clinically relevant morphology threshold for a disease state. In some embodiments, the method comprises determining a morphology state that when changed results in a change in a clinically relevant sign or symptom. In some embodiments, the clinically relevant sign or symptom is associated with the disease state.

Provided in some embodiments herein is a method for identifying a clinically relevant morphology or morphology threshold for a disease state, the method comprising determining a morphology state that when changed results in a change in a clinically relevant sign or symptom (e.g., associated with the disease state).

In some embodiments, a threshold for measuring a (e.g., physical, such as gland) morphology or morphology threshold is described elsewhere herein. In some embodiments, the threshold is illustrated by the figures provided herein. In some embodiments, the threshold is illustrated by the description of the figures provided herein. In some embodiments, threshold is an objective measure described herein. In some embodiments, threshold is associated with an objective measure described herein. In some embodiments, threshold is measured using an objective measure described herein.

In some embodiments, a clinically relevant sign or symptom is described elsewhere herein. In some embodiments, the clinically relevant sign or symptom is illustrated by the figures provided herein. In some embodiments, the clinically relevant sign or symptom is illustrated by the description of the figures provided herein. In some embodiments, the clinically relevant sign is an objective measure described herein. In some embodiments, the clinically relevant sign is associated with an objective measure described herein. In some embodiments, the clinically relevant sign is measured using an objective measure described herein.

In some embodiments, a morphology or morphology threshold for a disease state is described elsewhere herein. In some embodiments, the morphology or morphology threshold for a disease state is illustrated by the figures provided herein. In some embodiments, the morphology or morphology threshold for a disease state is illustrated by the description of the figures provided herein. In some embodiments, the morphology or morphology threshold for a disease state is open meibomian glands. In some embodiments, the morphology or morphology threshold for a disease state is meibum quality. In some embodiments, the morphology or morphology threshold for a disease state is meibum quantity.

In some embodiments, the clinically relevant sign or symptom is associated with a disease state.

Provided in some embodiments herein is a method for treating a disease state. In some embodiments, the method comprises evaluating a morphology state. In some embodiments, the morphology state is associated with the disease state. In some embodiments, the method comprises, determining whether the morphology state is above a threshold. In some embodiments, the method comprises, determining whether the morphology state is below a threshold. In some embodiments, the method comprises treating the disease state in an individual having an abnormal morphology state. In some embodiments, the abnormal morphology state is below the threshold. In some embodiment, the threshold is an MGS of 12. In some embodiment, the individual has an abnormal morphology state and the threshold is an MGS less than or equal to 12. In some embodiments, the abnormal morphology state is above the threshold.

Provided in some embodiments herein is a method for treating a disease state, the method comprising (i) evaluating a morphology state (e.g., associated with the disease state), (ii) determining whether the morphology state is above or below a threshold, and (iii) treating the disease state in an individual having an abnormal morphology state (e.g., wherein an abnormal morphology state is above or below the threshold).

In some embodiments, treating the disease state comprises administering a therapeutic agent for treating the disease state to the individual. In some embodiments, the therapeutic agent is a meibum enhancer (e.g., as described elsewhere herein). In some embodiments, the therapeutic agent is a keratolytic agent (e.g., as described elsewhere herein). In some embodiments, the therapeutic agent is selenium disulfide.

In some embodiments, the therapeutic agent (e.g., the meibum enhancer (e.g., the keratolytic agent)) is administered to or around the eye of the individual. In some embodiments, the therapeutic agent (e.g., the meibum enhancer (e.g., the keratolytic agent)) is administered to an eyelid margin of the individual.

Provided in some embodiments herein is a method for improving a sign or symptom of a disease state in an individual. In some embodiments, the sign is a clinically relevant sign (e.g., meibum quality, meibum quantity, or number of open or closed meibomian glands, such as measured by MGS). In some embodiments, the symptom is a clinically relevant symptom (e.g., ocular itching, such as measured by OSDI and/or VAS). In some embodiments, the method comprises scoring a morphological feature to determine a morphological score. In some embodiments, the morphological feature is associated with the disease state. In some embodiments, the method comprises determining if the morphological score is abnormal relative to a threshold score. In some embodiments, the method comprises providing a therapeutic agent to the individual. In some embodiments, the method comprises providing a therapeutic agent to the individual at least until a normal morphological score is achieved. In some embodiments, the normal morphological score is an MGS of greater than 12. In some embodiments, the therapeutic agent is useful for (treating) the disease state. In some embodiments, the therapeutic agent is described elsewhere herein, such as a meibum enhancer.

In some embodiments, the disease state is an ocular disorder. In some embodiments, the ocular disorder is described elsewhere herein.

In some embodiments, the disease state is meibomian gland dysfunction (MGD).

In some embodiments, the disease state is dry eye.

In some embodiments, the disease state is ocular itching.

In some embodiments, the disease state is blepharitis.

In some embodiments, the disease state is demodex. In some embodiments, the disease state is demodex infestation.

In some embodiments, the disease state is contact lens discomfort.

In some embodiments, the morphological feature is open meibomian glands.

In some embodiments, the morphological feature is meibum quality. In some embodiments, the morphological feature is a change in meibum quality.

In some embodiments, the morphological feature is meibum quantity. In some embodiments, the morphological feature is a change in meibum quantity.

In some embodiments, the threshold score is an MGS of 12.

In some embodiments, an MGS of greater than 12 is associated with a normal morphological state (e.g., of meibomian glands). In some embodiments, an MGS of less than or equal to 12 is associated with an abnormal morphological state (e.g., of meibomian glands).

In some embodiments, the disease state is an ocular disorder, the morphological feature is open meibomian glands, and the threshold score is an MGS of 12 (e.g., wherein >12 is a normal morphological state and ≤12 is an abnormal morphological state).

In some embodiments, the disease state is an ocular disorder, the morphological feature is (a change in) meibum quality, and the threshold score is an MGS of 12 (e.g., wherein >12 is a normal morphological state and ≤12 is an abnormal morphological state).

In some embodiments, a method described herein further comprises treating a disorder described herein until a change in an objective measure or symptom is achieved. In some embodiments, the change is an improvement.

In some embodiments, the objective measure is TBUT.

In some embodiments, the symptom is (measured using) OSDI. In some embodiments, the symptom is measured using OSDI, SPEED, VAS, or a subscale thereof. In some embodiments, the symptom is measured using OSDI. In some embodiments, the symptom is measured using SPEED. In some embodiments, the symptom is measured using VAS. In some embodiments, the symptom is measured using a subscale of OSDI, SPEED, and/or VAS.

Provided in some embodiments is a method for improving a sign or symptom of a disease state in an individual, the method comprising (i) scoring a morphological feature (e.g., associated with the disease state) to determine a morphological score, (ii) determining if the morphological score is abnormal relative to a threshold score; and providing a therapeutic agent (e.g., for the disease state) to the individual (e.g., at least until a normal morphological score is achieved).

Provided in some embodiments is a method for treating a first disease state in an individual. In some embodiments, the method comprises evaluating a morphological state to determine a morphological score. In some embodiments, the morphological score is indicative of a second disease state in an individual. In some embodiments, the method comprises comparing the morphological score to a morphological threshold score. In some embodiments, the method comprises determining if the morphological score is normal. In some embodiments, the method comprises determining if the morphological score is abnormal. In some embodiments, such as if the morphological score is abnormal, the method comprises providing a therapeutic agent described herein (e.g., a meibum enhancer) to the individual. In some embodiments, the method comprises treating the second disease state prior to treating the first disease state. In some embodiments, the method comprises treating the second disease state while treating the first disease state. In some embodiments, a therapeutic agent described herein (e.g., a meibum enhancer) is provided to the individual at least until the second disease state is treated. In some embodiments, a therapeutic agent described herein (e.g., a meibum enhancer) is provided to the individual at least until the first disease state is treated. In some embodiments, a therapeutic agent described herein (e.g., a meibum enhancer) is provided to the individual until the first and second disease states are treated.

Provided in some embodiments herein is a method for treating a first disease state in an individual, the method comprising (i) evaluating a morphological state to determine a morphological score indicative of a second disease state in an individual, (ii) comparing the morphological score to a morphological threshold score, (iii) determining if the morphological score is normal or abnormal, and (iv) treating the second disease state prior to or while treating the first disease state.

In some embodiments, a method described herein further comprises enriching a clinical trial population. In some embodiments, the clinical trial population includes individuals that (have been evaluated) to have an abnormal score. In some embodiments, the clinical trial population includes individuals that (have been evaluated) to have an abnormal morphological score.

In some embodiments, a method described herein further comprises treating one or more individual of a clinical trial population with a therapeutic agent during a clinical trial. In some embodiments, a method described herein further comprises treating one or more individual of a clinical trial population with a therapeutic agent before a clinical trial. In some embodiments, a method described herein further comprises treating one or more individual of a clinical trial population with a therapeutic agent after a clinical trial. In some embodiments, the clinical trial population includes individuals that (have been evaluated) to have an abnormal score. In some embodiments, the clinical trial population includes individuals that (have been evaluated) to have an abnormal morphological score.

Provided in some embodiments herein is a method for treating a first disease state in an individual. In some embodiments, the method comprises evaluating a morphological state to determine a morphological score indicative of a second disease state in an individual. In some embodiments, the method comprises comparing the morphological score to a morphological threshold score. In some embodiments, the method comprises determining if the morphological score is normal or abnormal. In some embodiments, the method comprises enriching a clinical trial population (e.g. the clinical trial population including individuals with an abnormal (morphological) score). In some embodiments, the method comprises. In some embodiments, the method comprises treating one or more individual of the clinical trial population with a therapeutic agent during or before a clinical trial.

In some embodiments, the first disease state is a first ocular disorder.

In some embodiments, the second disease state is a second ocular disorder. In some embodiments, the second ocular disorder is MGD.

In some embodiments, an individual described herein has had MGD for at least years, such as before receiving a treatment described herein.

In some embodiments, the morphological state is open meibomian glands. In some embodiments, the threshold score is an MGS of 12. In some embodiments, an MGS above 12 is associated with a normal morphological score. In some embodiments, an MGS below or equal to 12 is associated with an abnormal morphological score.

In some embodiments, the first disease state is a disease associated with a clinical trial. In some embodiments, the method is used to enhance a clinical trial population. In some embodiments, the method is used to improve clinical trial outcomes. In some embodiments, the first disease state is a disease associated with a clinical trial and the method is used to enhance a clinical trial population, such as to improve clinical trial outcomes. In some embodiments, the first disease state is a morphology score of a gland that predicts an inability to wear a lens (as designed or desired). In some embodiments, the first disease state is a change in a morphology score of a gland that predicts an inability to wear a lens (as designed or desired).

In some instances, primary efficacy measures of a study described herein (see Example 1) are divided into endpoints for primary efficacy signs and primary efficacy symptoms for MGD. In some instances, the primary efficacy sign for MGD is the change from baseline at month 3 in MGYLS (0 to 15 scale). In some embodiments, the individual has an improvement in MGYLS with continued use of the keratolytic agent (e.g., selenium sulfide) through 6-months. In some instances, a high MGYLS score is better than a low MGYLS score. In some instances, the primary efficacy symptom for MGD is the change from baseline at month 3 in OSDI total score (0 to 100 scale). In some embodiments, the individual has an improvement in OSDI total score with continued use of the keratolytic agent (e.g., selenium sulfide) through 6-months. In some instances, a low OSDI total score is better than a high OSDI total score.

In some embodiments, a method provided herein comprises evaluating the number of open meibomian glands of an individual (e.g., MGYLS score). In some instances, determining the MGYLS score is based on a technique for meibomian gland expression, such as where secretion in the lower eyelid of each eye is measured for five consecutive glands in each of three regions (e.g., temporal, central, and nasal). In some instances, the MGYLS is scored from 0-15, where lower scores indicate more severe disease. In some embodiments, a MGYLS responder is an individual who has an increase of ≥5 MGLYS (e.g., from baseline). In some embodiments, an individual receiving a composition described herein is a MGYLS responder.

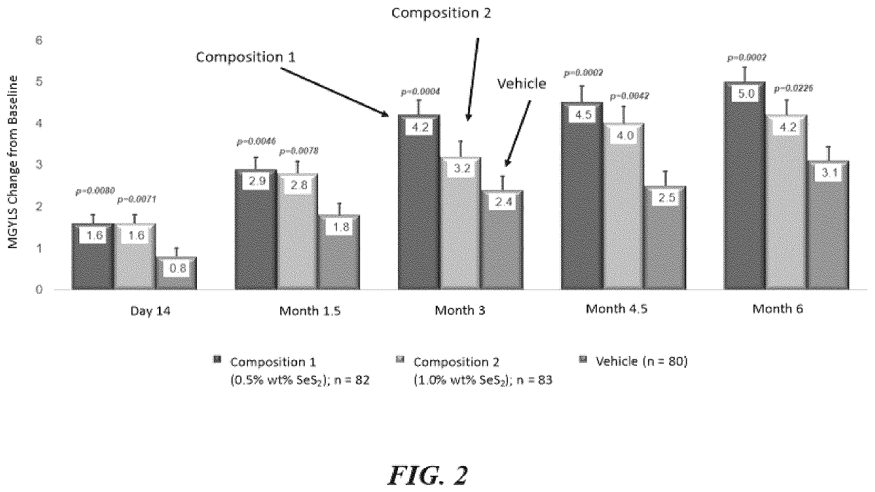

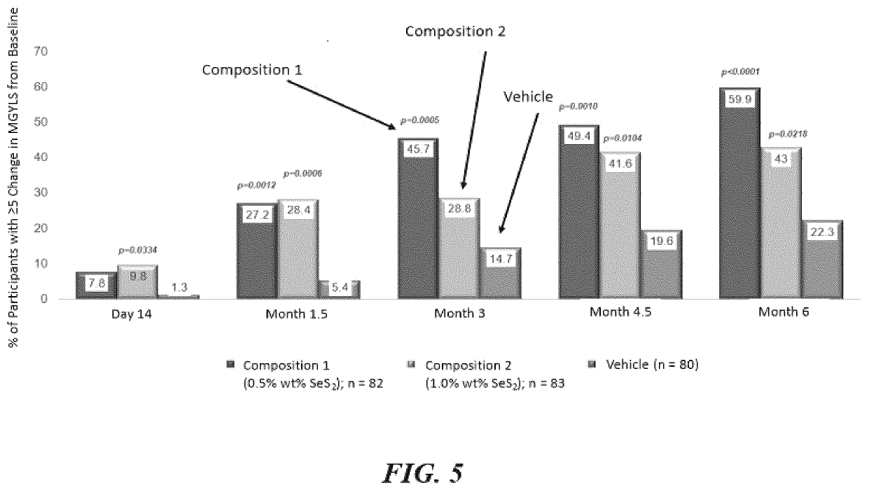

In some embodiments, a composition described herein (e.g., Composition 1 and Composition 2) provides a statistically significant improvement in an objective measure for an ocular indication (e.g., a sign) of an ocular disorder described herein. In some embodiments, such as described in Example 1, a composition described herein (e.g., Composition 1 and Composition 2) provides a statistically significant improvement in MGYLS change from baseline (e.g., selenium disulfide compared to vehicle). For example, FIG. 2 shows that Composition 1 and Composition 2 significantly improve change from baseline in MGYLS at day 14 day and month 1.5. FIG. 2 also shows that Composition 1 significantly improves change from baseline in MGYLS at month 3. Additionally, FIG. 2 and FIG. 5 show that the change in MGYLS continues to improve with continued use of Composition 1 and Composition 2 through 6-months. Moreover, while the vehicle provides an improvement in MGYLS over baseline, Composition 1 and Composition 2 provide significantly more improvement in MGYLS over baseline (compared to vehicle) at day 14, month 1.5, and/or month 3. In some instances, an improvement in MGYLS scores is associated with more open meibomian glands (e.g., compared to vehicle).

In some embodiments, a composition described herein (e.g., Composition 1 and/or Composition 2) provide an improvement (e.g., selenium disulfide compared to vehicle) in MGYLS scores (e.g., from baseline to month 3, month 4.5, and/or month 6 of treatment) of at least 3 (e.g., at least 3.25, at least 3.5, at least 3.75, at least 4, at least 4.25). In some embodiments, a composition described herein (e.g., Composition 1 and/or Composition 2) provide an improvement (e.g., selenium disulfide compared to vehicle) in MGYLS scores (e.g., from baseline to month 3, month 4.5, and/or month 6 of treatment) of at most 6 (e.g., at most 5.5, at most 5, at most 4.5, at most 4, at most 3.5). In some embodiments, a composition described herein (e.g., Composition 1 and/or Composition 2) provide an improvement (e.g., selenium disulfide compared to vehicle) in MGYLS scores (e.g., from baseline to month 3 of treatment) of about 4.2. In some embodiments, a composition described herein (e.g., Composition 1 and/or Composition 2) provides an improvement (e.g., selenium disulfide compared to vehicle) in MGYLS scores (e.g., from baseline to month 4.5 of treatment) of about 4.5. In some embodiments, a composition described herein (e.g., Composition 1 and/or Composition 2) provide an improvement (e.g., selenium disulfide compared to vehicle) in MGYLS scores (e.g., from baseline to month 6 of treatment) of about 5.0. In some embodiments, a composition described herein (e.g., Composition 1 and/or Composition 2) provide an improvement (e.g., selenium disulfide compared to vehicle) in MGYLS scores (e.g., from baseline to month 3 of treatment) of about 3.2. In some embodiments, a composition described herein (e.g., Composition 1 and/or Composition 2) provides an improvement (e.g., selenium disulfide compared to vehicle) in MGYLS scores (e.g., from baseline to month 4.5 of treatment) of about 4.0. In some embodiments, a composition described herein (e.g., Composition 1 and/or Composition 2) provide an improvement (e.g., selenium disulfide compared to vehicle) in MGYLS scores (e.g., from baseline to month 6 of treatment) of about 4.2.

In some embodiments, an individual provided herein has an MGYLS score of at least 0.5 (e.g., at least 1, at least 1.5, at least 2), such as before treatment with a composition described herein (e.g., Composition 1 and/or Composition 2). In some embodiments, an individual provided herein has an MGYLS score of at most 2.5 (e.g., at most 2.25, at most 2, at most 1.75, at most 1.5), such as before treatment with a composition described herein (e.g., Composition 1 and/or Composition 2). In some embodiments, an individual provided herein has an MGYLS score of about 1.8, such as before treatment with a composition described herein (e.g., Composition 1 and/or Composition 2).

In some instances, a composition described herein (e.g., Composition 1 and/or Composition 2) provide a (e.g., statistically significant) increase in the number of open meibomian glands, such as in individuals classified as MGYLS responders. In some instances, a composition provided herein (e.g., Composition 1 and/or Composition 2) provide an improvement in MGYLS scores from baseline to 14 days and 1.5 months after treatment, see FIG. 2. In some instances, a composition provided herein (e.g., Composition 1 and/or Composition 2) provides an improvement in MGYLS scores from baseline to 4.5 months and 6 months after treatment, see FIG. 2. In some instances, significant number of individuals receiving a composition provided herein (e.g., Composition 1 and/or Composition 2) were MGYLS responders at 14 days, 1.5 months, and 3 months after treatment, see FIG. 5. In some instances, a significant number of individuals receiving Composition 1 or Composition 2 were MGYLS responders at 14 days, 1.5 months, 3 months, 4.5 months, and/or 6 months after treatment, see FIG. 5.

In some embodiments, an individual is an OSDI responder when their OSDI total score is less than 13. In some instances, a total OSDI score of less than 13 is considered normal or asymptomatic, such as for dry eye disease. In some instances, an individual provided herein is an OSDI responder.

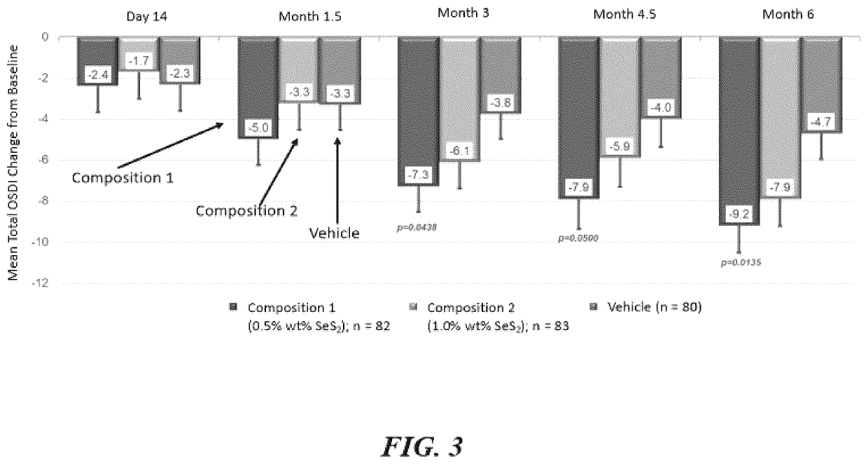

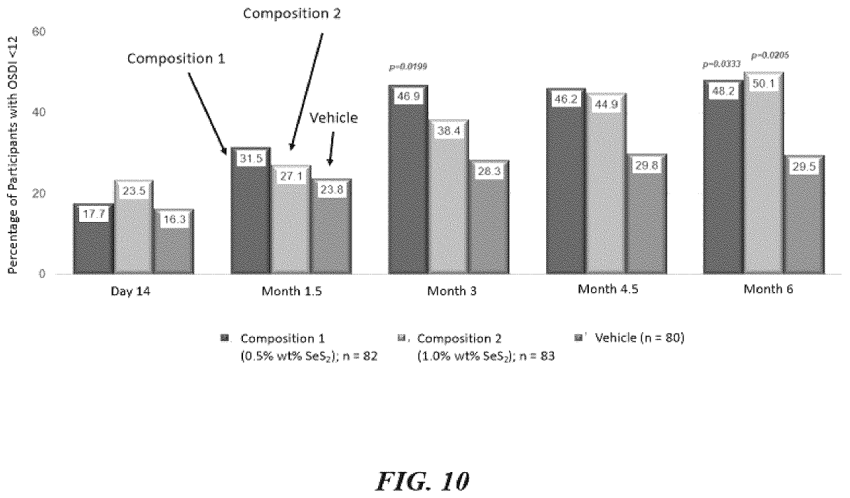

In some embodiments, a composition described herein (e.g., Composition 1) provides a statistically significant improvement in a symptom for an ocular indication of an ocular disorder described herein. In some embodiments, a composition described herein (e.g., Composition 1) provides a statistically significant improvement in total OSDI change from baseline (e.g., selenium disulfide compared to vehicle). For example, FIG. 3 shows that Composition 1 significantly improves change from baseline in total OSDI at month 3. Additionally, FIG. 3 and FIG. 10 show that Composition 1 significantly improves change from baseline in total OSDI at months 4.5 and 6. Also, FIG. 3 and FIG. 10 show that Composition 2 significantly improves change from baseline in total OSDI at month 6.

In some embodiments, a composition described herein (e.g., Composition 1 and/or Composition 2) improves OSDI total scores from baseline to month three of treatment (e.g., as compared to vehicle), see FIG. 3. In some embodiments, a composition provided herein (e.g., Composition 1 and/or Composition 2) provides an improvement in OSDI total scores from baseline to 4.5 months and 6 months after treatment, see FIG. 3. In some instances, greater OSDI total score improvement is associated with greater symptom relief, such as compared to vehicle. In some embodiments, a composition described herein (e.g., Composition 1 and/or Composition 2) improves OSDI total scores by at least 5 (e.g., at least 5.5, at least 6, at least 6.5, at least 7, at least 7.5) from baseline, such as after 3 months of treatment. In some embodiments, a composition described herein (e.g., Composition 1 and/or Composition 2) improves OSDI total scores by at most 9 (e.g., at most 8.5, at most 8, at most 7.5, at most 7, at most 6.5) from baseline, such as after 3 months of treatment. In some embodiments, a composition described herein (e.g., Composition 1 and/or Composition 2) improves OSDI total scores by at most 12 (e.g., at most 11.5, at most 11, at most 10.5, at most 10, at most 9.5) from baseline, such as after 6 months of treatment. In some embodiments, a composition described herein (e.g., Composition 1 and/or Composition 2) improves OSDI total scores by about 7.3 from baseline, such as after 3 months of treatment. In some embodiments, a composition described herein (e.g., Composition 1 and/or Composition 2) improves OSDI total scores by about 7.9 from baseline, such as after 4.5 months of treatment. In some embodiments, a composition described herein (e.g., Composition 1 and/or Composition 2) improves OSDI total scores by about 9.2 from baseline, such as after 6 months of treatment. In some embodiments, a composition described herein (e.g., Composition 1 and/or Composition 2) improves OSDI total scores by about 6.1 from baseline, such as after 3 months of treatment. In some embodiments, a composition described herein (e.g., Composition 1 and/or Composition 2) improves OSDI total scores by about 5.9 from baseline, such as after 4.5 months of treatment. In some embodiments, a composition described herein (e.g., Composition 1 and/or Composition 2) improves OSDI total scores by about 7.9 from baseline, such as after 6 months of treatment.

In some instances, a composition described herein (e.g., Composition 1 and/or Composition 2) provides a (e.g., statistically significant) decrease in OSDI total scores. In some instances, an individual with a decrease in OSDI total scores is (significantly more) asymptomatic after treatment (e.g., compared to vehicle). In some embodiments, a composition provided herein (e.g., Composition 1 and/or Composition 2) provide higher OSDI responder rates after treatment (e.g., compared to vehicle), see FIG. 12.

In some embodiments, an individual provided herein has an OSDI total score of at least 20 (e.g., at least 22, at least 24, at least 26, at least 28), such as before treatment. In some embodiments, an individual provided herein has an OSDI total score of at most 35 (e.g., at most 30, at most 25, at most 20), such as before treatment. In some embodiments, an individual provided herein has an OSDI total score of about 33, such as before treatment. In some embodiments, an individual provided herein has an OSDI total score of about 25, such as before treatment. In some embodiments, an individual provided herein has an OSDI total score of about 13-33, such as before treatment.

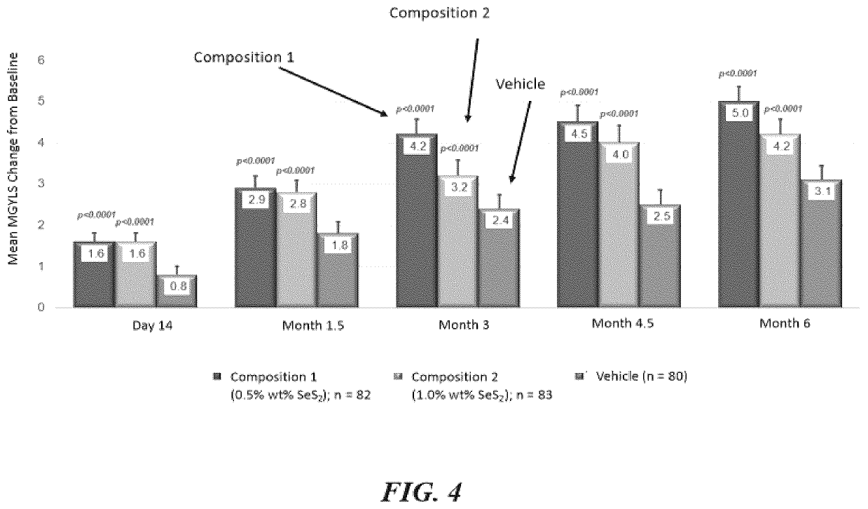

In some embodiments, a composition described herein (e.g., Composition 1) is useful for opening obstructed (meibomian) glands. In some embodiments, a composition described herein (e.g., Composition 1) demonstrates statistically significant and clinically meaningful benefit over vehicle in opening meibomian glands. For example, FIG. 4 and FIG. 5 show that 45.7% (p=0.0005 compared to vehicle at month 3 of treatment) of individuals who received Composition 1 had their glands opened to a normal level (MGYLS responder rate) with an improvement from baseline of 4.2 glands (p<0.0001—as compared to baseline at month 3 of treatment). FIG. 4 and FIG. 5 also show that 49.4% (p=0.0010—compared to vehicle at month 4.5 of treatment) of individuals who received Composition 1 had their glands opened to a normal level (MGYLS responder rate) with an improvement from baseline of 4.5 glands (p<0.0001—as compared to baseline at month 4.5 of treatment). FIG. 4 and FIG. 5 also show that 59.9% (p<0.0001-compared to vehicle at month 6 of treatment) of individuals who received Composition 1 had their glands opened to a normal level (MGYLS responder rate) with an improvement from baseline of 5.0 glands (p<0.0001—as compared to baseline at month 6 of treatment). FIG. 4 and FIG. 5 also show that 28.8% (p=0.0005-compared to vehicle at month 3 of treatment) of individuals who received Composition 2 had their glands opened to a normal level (MGYLS responder rate) with an improvement from baseline of 3.2 glands (p<0.0001—as compared to baseline at month 3 of treatment). FIG. 4 and FIG. 5 also show that 41.6% (p=0.0005-compared to vehicle at month 4.5 of treatment) of individuals who received Composition 2 had their glands opened to a normal level (MGYLS responder rate) with an improvement from baseline of 4.0 glands (p<0.0104—as compared to baseline at month 4.5 of treatment). Moreover, about 60% of individuals of the study described in Example 1 had their glands opened to a normal level, such as within 6 months of starting treatment. FIG. 4 and FIG. 5 also show that 43% (p=0.0218-compared to vehicle at month 6 of treatment) of individuals who received Composition 2 had their glands opened to a normal level (MGYLS responder rate) with an improvement from baseline of 4.2 glands (p<0.0001—as compared to baseline at month 6 of treatment). Overall, about 60% of individuals of the study described in Example 1 had their glands opened to a normal level, such as within 6 months of starting treatment. In some embodiments, MGYLS responders are a percentage of individuals with a meaningful increase in the number of open glands.

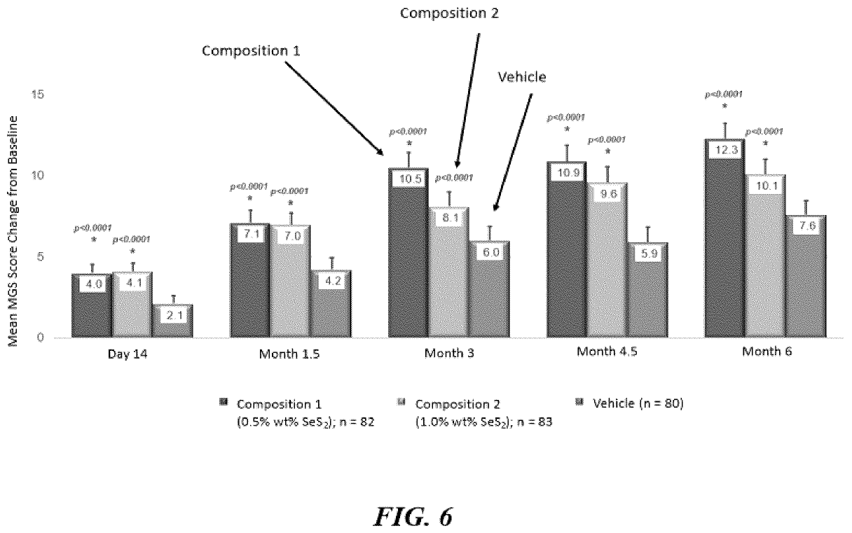

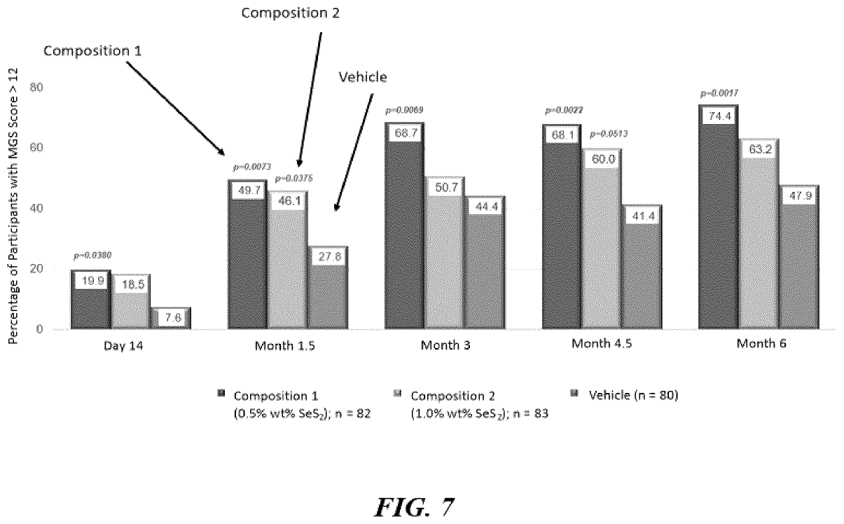

In some embodiments, a composition described herein (e.g., Composition 1) is useful for improving meibum quality and/or quantity. In some embodiments, a composition described herein (e.g., Composition 1) demonstrates statistically significant and clinically meaningful benefit over vehicle in improving meibum quality and quantity. For example, FIG. 6 and FIG. 7 show that 68.7% (p=0.0069-compared to vehicle at month 3 of treatment) of individuals who received Composition 1 had their meibum quality return to normal levels (MGS responder rate) with an improvement from baseline of 10.5 (p<0.0001—as compared to baseline at month 3 of treatment). FIG. 6 and FIG. 7 also show that 50.7% (p=0.0069-compared to vehicle at month 3 of treatment) of individuals who received Composition 1 had their meibum quality return to normal levels (MGS responder rate) with an improvement from baseline of 8.0 (p<0.0001—as compared to baseline at month 3 of treatment). FIG. 6 and FIG. 7 also show that 68.1% (p=0.0022-compared to vehicle at month 4.5 of treatment) of individuals who received Composition 1 had their meibum quality return to normal levels (MGS responder rate) with an improvement from baseline of 10.9 (p<0.0001—as compared to baseline at month 4.5 of treatment). FIG. 6 and FIG. 7 also show that 74.4% (p=0.0017-compared to vehicle at month 6 of treatment) of individuals who received Composition 1 had their meibum quality return to normal levels (MGS responder rate) with an improvement from baseline of 12.3 (p<0.0001—as compared to baseline at month 6 of treatment). FIG. 6 and FIG. 7 also show that 60.0% (p=0.0513-compared to vehicle at month 4.5 of treatment) of individuals who received Composition 2 had their meibum quality return to normal levels (MGS responder rate) with an improvement from baseline of 9.6 (p<0.0001—as compared to baseline at month 4.5 of treatment). Overall, up to about 75% of individuals of the study described in Example 1 had their meibum quality return to a normal level, such as within 6 months of starting treatment. In some embodiments, MGS responders are a percentage of individuals whose meibum returned to normal.

In some embodiments, an individual described herein has an MGS score less than or equal to 12, such as before treatment. In some embodiments, an individual described herein has an MGS score of 6 to 12, such as before treatment. In some embodiments, an individual described herein has an MGS score described herein in both eyes, such as before treatment.

In some embodiments, a composition described herein (e.g., Composition 1 and Composition 2) provides significantly greater improvement in MGS scores compared to vehicle, such as after 14 days, 1.5 months, 3 months, 4.5 months, and 6 months of treatment. In some embodiments, a composition described herein (e.g., Composition 1 and Composition 2) provides for a higher percentage of MGS responders (e.g., individuals with a ‘normal’ value of MGS>12) in comparison to vehicle, see FIG. 7. In some embodiments, a composition provided herein (e.g., Composition 1 or Composition 2) is useful for providing ‘normal’ meibum quality. In some embodiments, a composition described herein (e.g., Composition 1) is useful for opening obstructed meibomian glands and substantially improving meibum quality and/or quantity.

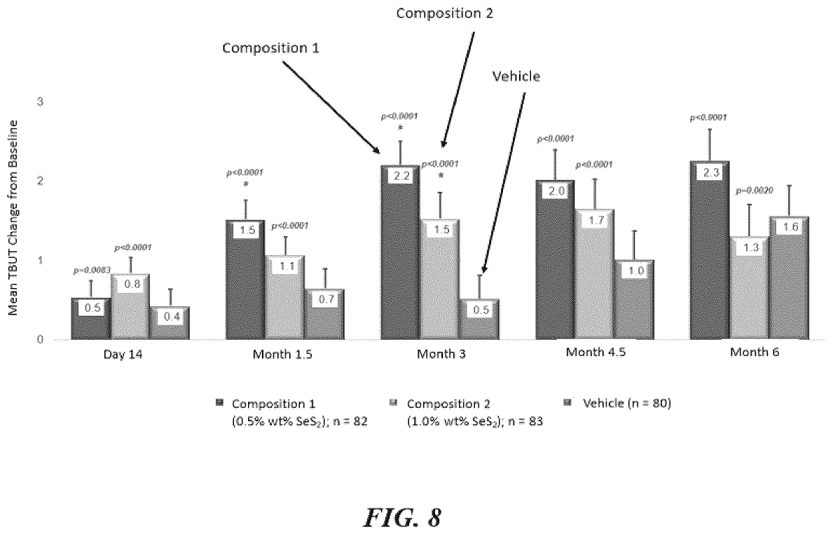

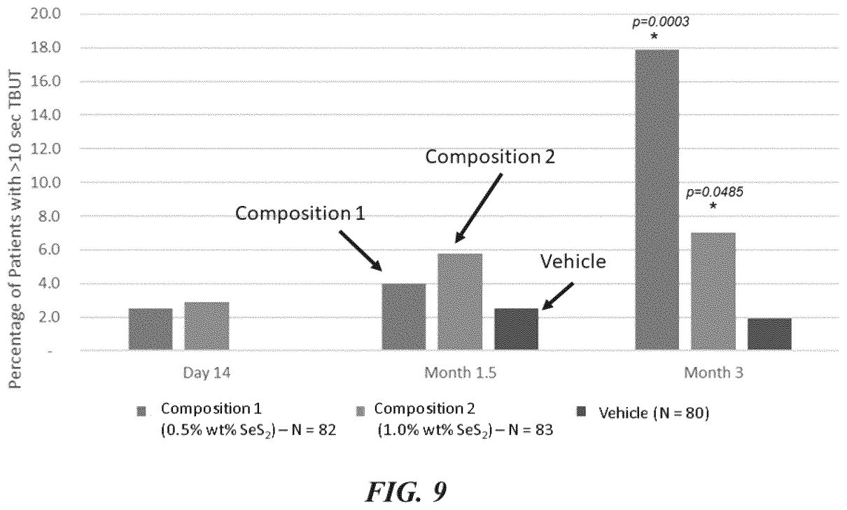

In some embodiments, a composition described herein (e.g., Composition 1) is useful for improving tear quality. In some embodiments, a composition described herein (e.g., Composition 1) demonstrates improvement over vehicle in tear stability. In some embodiments, a composition described herein (e.g., Composition 1) demonstrates improvement over vehicle in tear film stability, meibomian gland secretion, and ocular surface symptoms. In some embodiments, a composition described herein (e.g., Composition 1) demonstrates improvement over vehicle in tear break up time (TBUT) (which can be a tear stability measure). In some embodiments, a composition described herein (e.g., Composition 1) significantly improved meibomian gland secretions and tear film stability (compared to vehicle), in some instances, providing clinically significant improvements in ocular symptoms for 6 months of treatment. In some embodiments, a composition described herein (e.g., Composition 1) demonstrates an improvement from baseline. For example, FIG. 8 shows that individuals receiving Composition 1 had an improvement from baseline of 2.2 seconds (p<0.0001—as compared to baseline at month 3 of treatment). FIG. 8 also shows that individuals receiving Composition 1 had an improvement from baseline of 2.0 and 2.3 seconds (p<0.0001—as compared to baseline at months 4.5 and 6 of treatment, respectively). Also, FIG. 8 shows that individuals receiving Composition 2 had an improvement from baseline of 1.5 seconds (p<0.0001—as compared to baseline at month 3 of treatment). Additionally, FIG. 9 shows that 17.9% of individuals who received Composition 1 had normal tear film stability of greater than 10 sec TBUT (p<0.0003—as compared to vehicle at month 3 of treatment). Moreover, FIG. 9 shows that 7% of individuals who received Composition 2 had normal tear film stability of greater than 10 sec TBUT (p<0.0003—as compared to vehicle at month 3 of treatment). Overall, individuals receiving Composition 1 had more than a 2 second improvement in TBUT maintained from Month 3 onward (see FIG. 8). In some embodiments, TBUT responders are a percentage of individuals with a meaningful increase in TBUT.

In some instances, tear film stability (as measured by TBUT) significantly increased from baseline in both treatment groups compared with vehicle at month 3 (Composition 1 mean change=2.2 s, p<0.0001 vs vehicle; Composition 2=1.5 s, p=0.0187 vs vehicle; vehicle=0.5 s), with increases generally sustained at month 6 but no longer significant vs vehicle (Composition 1=2.3 s; Composition 2=1.3 s; vehicle=1.6 s; p>0.05 vs vehicle for both concentrations).

In some instances, TBUT is the time it takes, such as in seconds, for a first dry spot to appear on the cornea after a complete blink. In some instances, increased TBUT is associated with improvement of symptoms. In some embodiments, a TBUT responder is an individual with a TBUT of greater than or equal to 10 seconds (e.g., which is considered normal). In some embodiments, an individual described herein is a TBUT responder.

In some embodiments, a composition described herein (e.g., Composition 1 and/or Composition 2) improves TBUT, such as after treatment for 14 days, 1.5 months, 3 months, 4.5 months, and/or 6 months, such as compared to vehicle. In some embodiments, a significantly higher percentage of individuals achieve normal TBUT (e.g., less than or equal to 10 seconds (e.g., are TBUT responders) at month 3 compared to vehicle) after receiving a composition described herein (e.g. Composition 1 and/or Composition 2), see FIG. 9.

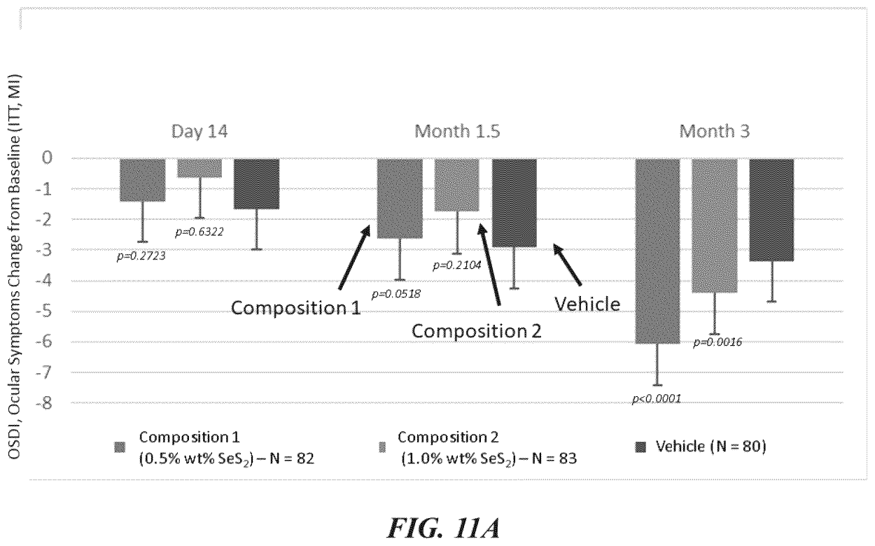

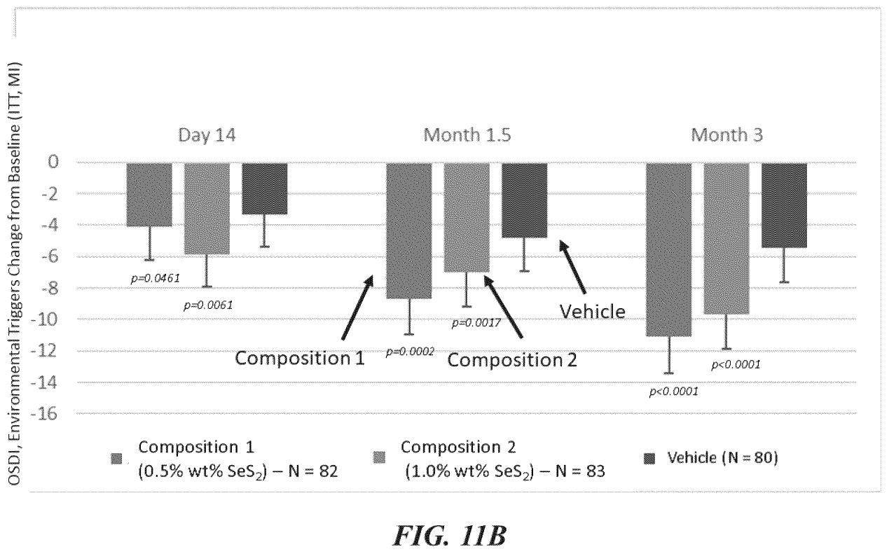

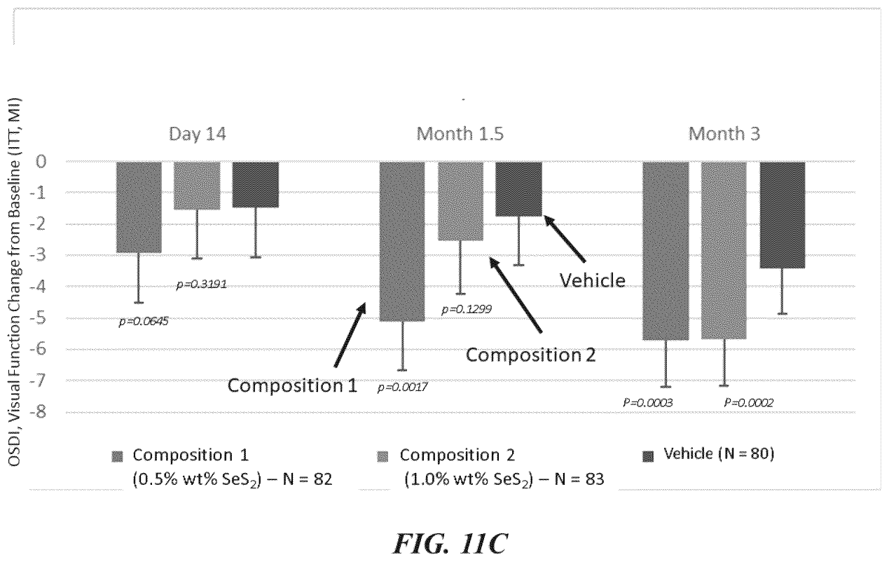

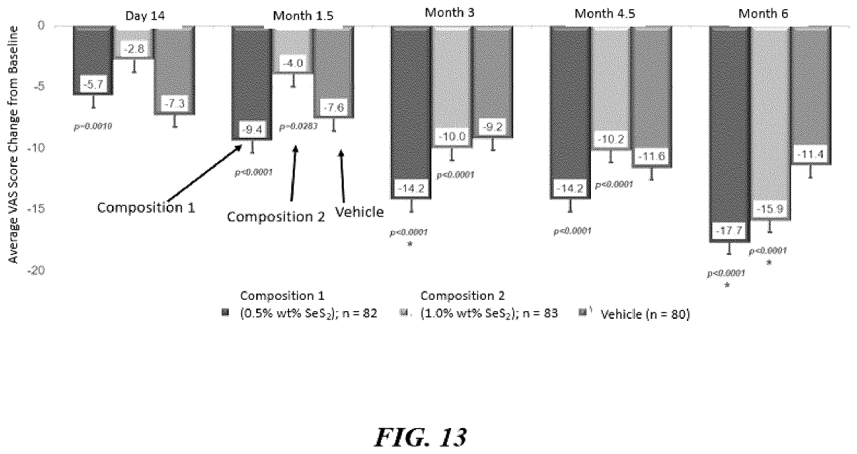

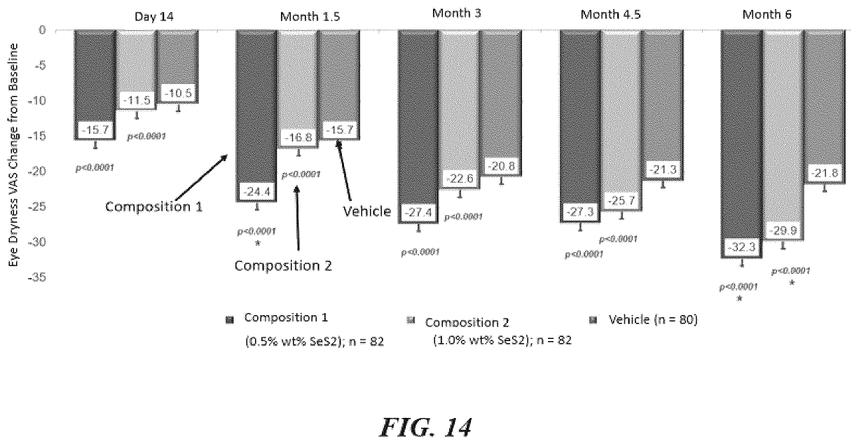

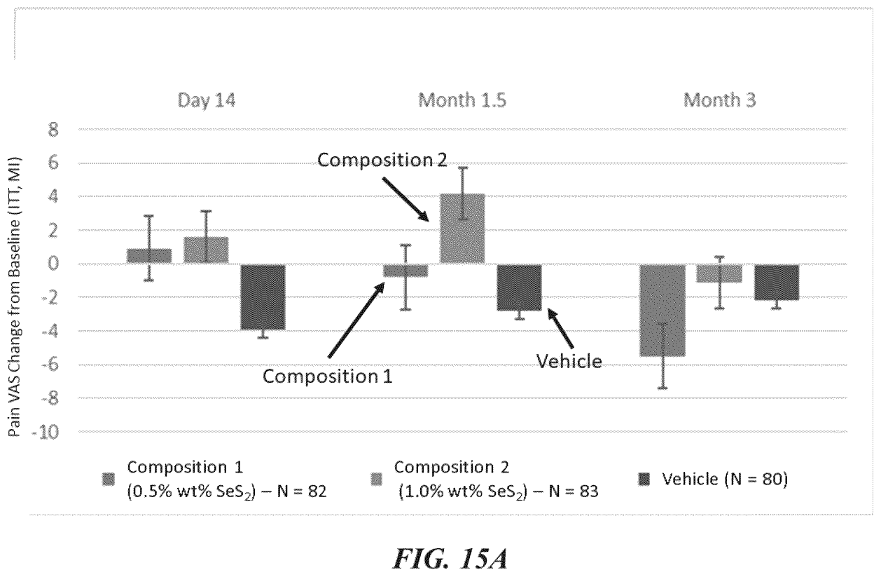

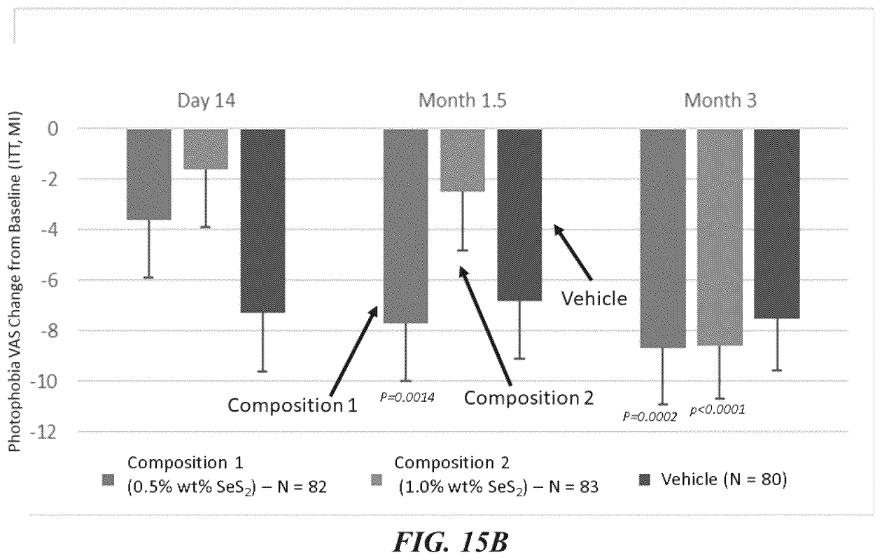

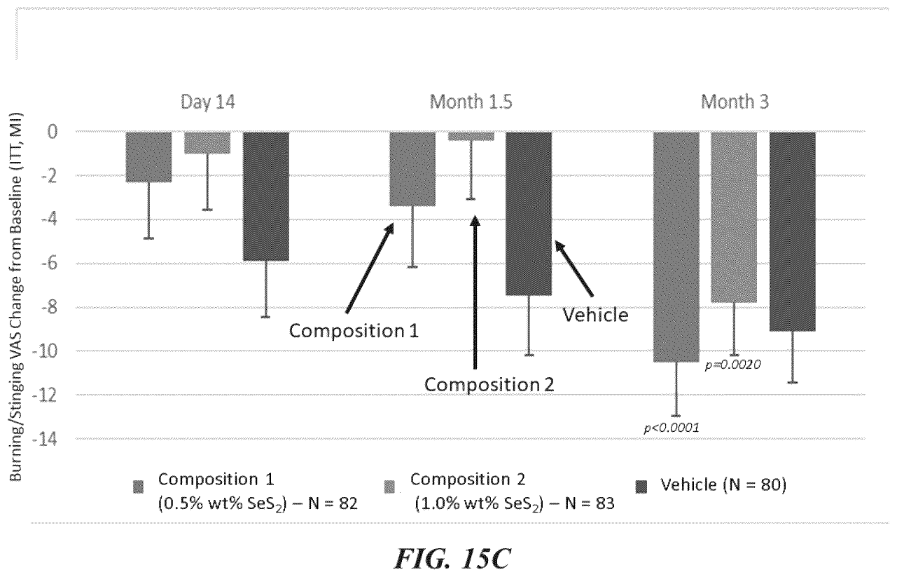

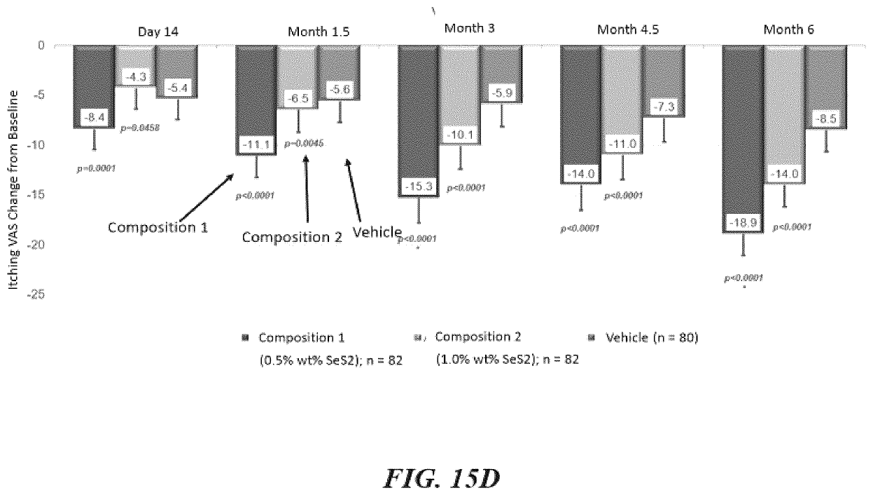

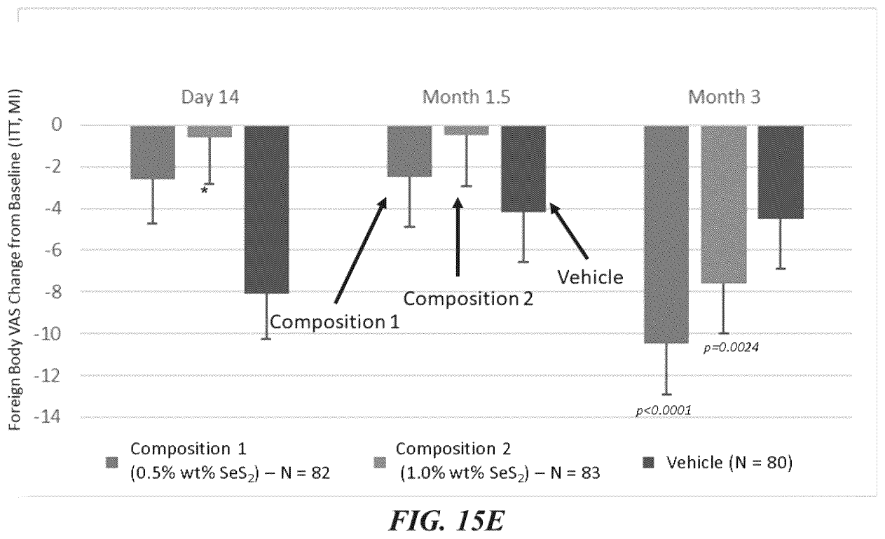

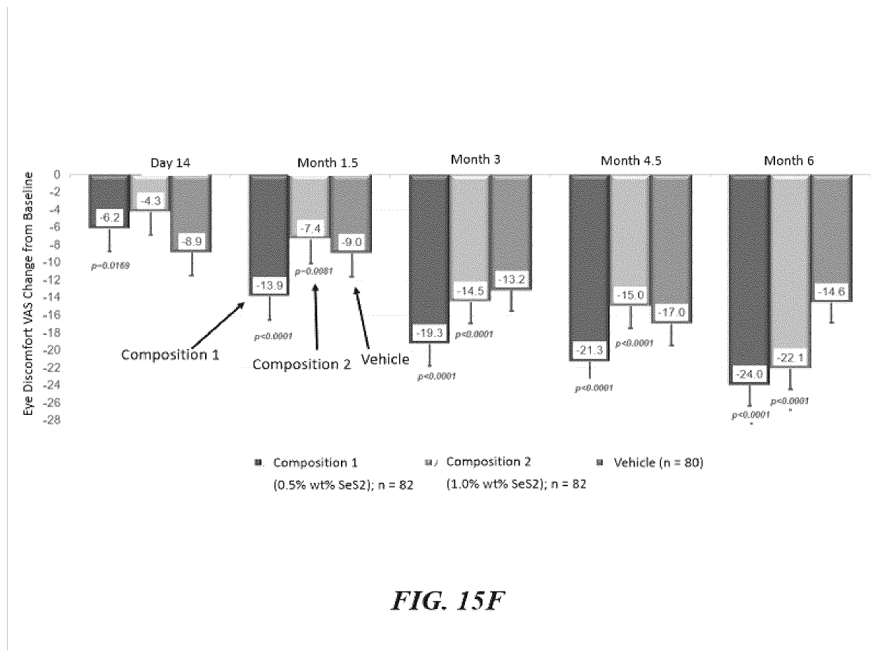

In some embodiments, a composition described herein (e.g., Composition 1) is useful for improving a symptom measure. In some embodiments, a composition described herein (e.g., Composition 1) demonstrates statistically significant and clinically meaningful benefit over vehicle (placebo) in total OSDI, SPEED, ocular itch, eye discomfort, and/or eye dryness. For example, FIG. 10 shows that 46.9% of individuals who received Composition 1 became asymptomatic on Total OSDI (OSDI responder rate) (p=0.0199—as compared to vehicle at month 3 of treatment). FIG. 10 also shows that 48.2% of individuals who received Composition 1 became asymptomatic on Total OSDI (OSDI responder rate) (p=0.0333—as compared to vehicle at month 6 of treatment). FIG. 10 also shows that 50.1% of individuals who received Composition 2 became asymptomatic on Total OSDI (OSDI responder rate) (p=0.0205—as compared to vehicle at month 6 of treatment). Overall, up to about 50% of individuals of the study described in Example 1 became asymptomatic (e.g., as measured by total OSDI), such as within 6 months of starting treatment. In some embodiments, OSDI responders are a percentage of individuals who receive a normal grade. Moreover, FIG. 11A, FIG. 11B, and FIG. 11C show that subscales of OSDI (e.g., ocular symptoms change from baseline, environmental triggers change from baseline, and visual function change from baseline) are consistent with total OSDI, statistically significant, and clinically meaningful compared to baseline. Additionally, FIG. 12 shows that individuals receiving Composition 1 and Composition 2 had improvement over vehicle (placebo) in self-reported outcome measures of SPEED (p<0.0001—as compared to baseline at month 3, month 4.5, and month 6 of treatment) and A verage VA S (visual analogue scale) (p<0.0001—as compared to baseline at month 3, month 4.5, and month 6 of treatment). In some embodiments, a composition described herein (e.g., Composition 1 and Composition 2) decreases SPEED scores in an individual described herein. Furthermore, individuals receiving Composition 1 had improvement over vehicle (placebo) in reported outcome measures (e.g., FIG. 13), such as of eye dryness (p<0.0001—as compared to baseline at month 3 of treatment, see FIG. 14) and ocular itch (visual analogue scale) (p<0.0001—as compared to baseline at month 3 of treatment, see FIG. 15D). Moreover, individuals receiving Composition 1 or Composition 2 had improvement over vehicle (placebo) in reported outcome measures at Month 6 of treatment (e.g., FIG. 13). In some instances, such as shown in FIGS. 15A-15F reported outcome measures include but are not limited to eye dryness, ocular itching, pain, photophobia, burning, stinging, foreign body, and/or eye discomfort. For Example, individuals receiving Composition 1 had significant improvement over vehicle (placebo) in reported outcome measures of ocular itching (FIG. 15.D), eye discomfort (FIG. 15F), and eye dryness (FIG. 14) at Month 6 of treatment. Individuals receiving Composition 2 had significant improvement over vehicle (placebo) in reported outcome measures of eye discomfort (FIG. 15F) and eye dryness (FIG. 14) at Month 6 of treatment.

As evidenced by FIGS. 2, 4-7 as well as FIGS. 3, 15A, 15D, and 15E, while a significant improvement in the sign (e.g., MGYLS) can be observed as early as day 14, an improvement in the symptom (e.g., total OSDI) may not be observed until month 3. Additionally, while FIGS. 2 and 4-7 show that a vehicle can provide an improvement to the sign (e.g., MGYLS, MGS, and TBUT), FIGS. 3, 15A, 15D, and 15E show that a vehicle may not provide an improvement to the symptom (e.g., total OSDI), suggesting that improving a sign (e.g., MGYLS) doesn't necessarily provide, correlate to, or correspond to an improvement in a symptom. In fact, FIGS. 14, 15A, 15D, 15E, and 15F show that while a vehicle may provide an initial (e.g., at day 14) improvement in a symptom (e.g., pain), over time (e.g., at month 1.5 and month 3 and month 4.5 and month 6) an increase in the symptom (compared to at day 14) may occur. Contrarily, while a composition described herein (e.g., Composition 1 or Composition 2) may not improve symptoms, or even increase symptoms, initially (e.g., see FIGS. 14, 15A, 15D, 15E, and 15F), maintaining use of the composition can provide a significant improvement in the symptom(s) over time, such as at month 1.5 and/or month 3, or even as long as month 4.5 and/or month 6 (e.g., see FIGS. 2, 4-7, 10, 13, 14 15D, and 15F).

In some instances, one or more meibomian gland of an individual receiving a composition described herein (e.g., Composition 1) has a change in morphology of the one or more meibomian gland. In some instances, a change in morphology of the one or more meibomian gland provides, such as after a period of time (e.g., after 2 weeks or more of selenium disulfide treatment (e.g., at or after month 1.5 and/or at or after month 3 and/or at or after month 4.5 and/or at or after month 6 of selenium disulfide treatment)).

In some embodiments, an individual receiving a composition described herein (e.g., Composition 1) has an improvement in one or more objective measure (e.g., a sign), such as for an ocular indication. In some embodiments, an individual receiving a composition described herein (e.g., Composition 1) has an improvement in one or more symptom, such as for an ocular indication. In some embodiments, an individual receiving a composition described herein (e.g., Composition 1) has an improvement in one or more objective measure (e.g., a sign) and one or more symptom, such as for an ocular indication.

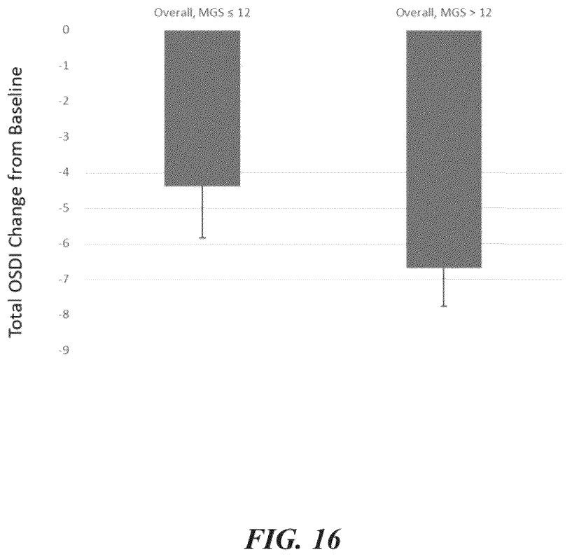

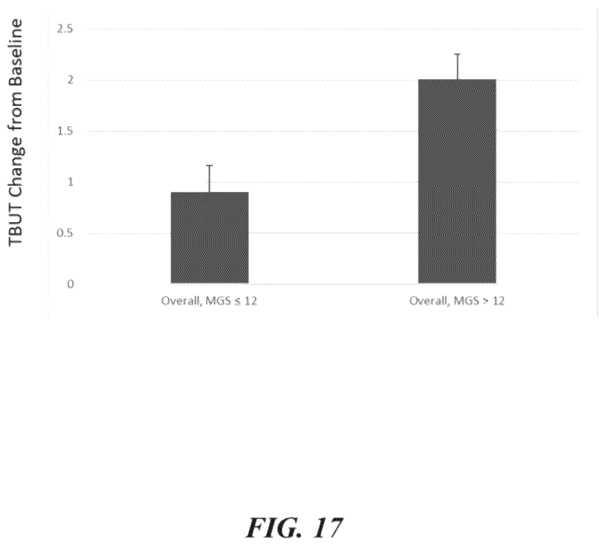

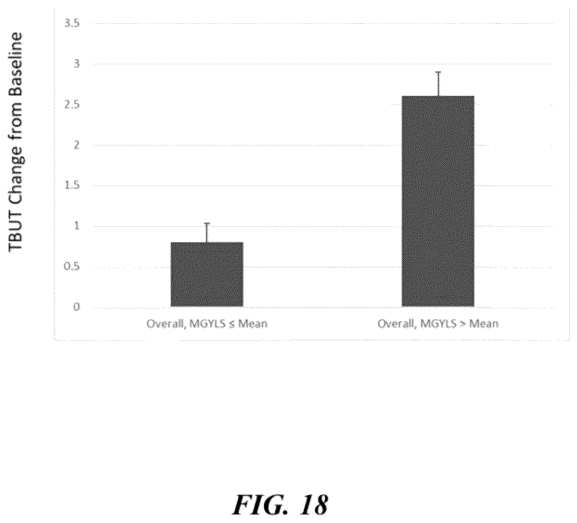

In some instances, improvement in one or more objective measure (e.g., a sign) and one or more symptom is evidenced by FIGS. 16-18. For example, FIGS. 16-18 demonstrate that an increase in open meibomian glands (e.g., MGYLS) is associated with improvement in TBUT and an increase in meibum quality (e.g., MGS) is associated with improvements in both TBUT and Total OSDI (e.g., symptoms). Moreover, FIGS. 16-18 support the clinical relevance of a responder criteria for a proportion of patients achieving an MGS of greater than 12 at month 3, for example, as it is associated with a significant improvement in how patients feel (Total OSDI) and downstream signs, such as TBUT. In some instances, a MGS responder is an individual with a post-randomization MGS score greater than 12. In some instances, such as at month 3 of selenium disulfide treatment (for the total population of individuals in the study described in Example 1), MGS scores greater than 12 were associated with significantly more improvement on Total OSDI than MGS scores less than or equal to 12 (e.g., Example 1, expansion cohort), see FIG. 16. In some instances, such as at month 3 of selenium disulfide treatment (for the total population of individuals in the study described in Example 1), MGS scores greater than 12 were associated with significantly longer TBUT than MGS scores less than or equal to 12 (e.g., Example 1, expansion cohort), see FIG. 17. In some instances, such as at month 3 of selenium disulfide treatment (for the total population of individuals in the study described in Example 1), MGYLS scores greater than the mean MGYLS scores were associated with significantly longer TBUT than MGYLS scores less than or equal the mean MGYLS scores (e.g., Example 1, expansion cohort), see FIG. 18.

Additionally, as evidenced by FIG. 16-18 and described elsewhere herein, provided in some instances herein is a method for identifying a clinically relevant sign or symptom in an individual or for a disease state.

Additionally, as evidenced by FIG. 16-18 and described elsewhere herein, provided in some instances herein is a method for using a morphology threshold measure (e.g., a physical morphology threshold measure, such as a physical morphology threshold measure (e.g., MGS)) to predict a change in a clinically relevant sign or a symptom in an individual (or for a disease state).

Additionally, as evidenced by FIG. 16-18 and described elsewhere herein, provided in some instances herein is a method for identifying a clinically relevant sign or symptom in an individual or for a disease state.

Additionally, as evidenced by FIG. 16-18 and described elsewhere herein, provided in some instances herein is a method for identifying a clinically relevant morphology or morphology threshold for a disease state.

Additionally, as evidenced by FIG. 16-18 and described elsewhere herein, provided in some instances herein is a method for enriching a patient population, such as a clinical trial population.

In some instances, an individual described herein has one or more sign (e.g., as determined by an objective measure) of an ocular indication after a period of time. In some instances, an individual described herein has one or more symptom of an ocular indication after a period of time. In some instances, an individual described herein has one or more sign (e.g., as determined by an objective measure) and one or more symptom of an ocular indication after a period of time.

In some embodiments, an individual has improvement in the one or more sign (e.g., as determined by an objective measure) of an ocular indication and/or one or more symptom of an ocular indication within 3 months (e.g., 2.5 months, 2 months, 1.5 months, 1 month, 3 weeks, 14 days, at least 10 days, at least 7 days) of administration of a composition described herein (e.g., Composition 1 or Composition 2). In some embodiments, an individual has improvement in the one or more sign (e.g., as determined by an objective measure) of an ocular indication and/or one or more symptom of an ocular indication at least 7 days (e.g., 10 days or more, 14 days or more, 30 days or more, 1.5 months or more, or 3 months or more) after administration of a composition described herein (e.g., Composition 1 or Composition 2). In some instances, the individual has improvement in the one or more sign within 3 months of administration of a composition described herein (e.g., Composition 1 or Composition 2). In some instances, the individual has improvement in the one or more sign within 1.5 months of administration of a composition described herein (e.g., Composition 1 or Composition 2). In some instances, the individual has improvement in the one or more sign within 14 days of administration of a composition described herein (e.g., Composition 1 or Composition 2). In some instances, the individual has improvement in the one or more symptom within 3 months of administration of a composition described herein (e.g., Composition 1 or Composition 2). In some instances, the individual has improvement in the one or more symptom within 1.5 months of administration of a composition described herein (e.g., Composition 1 or Composition 2). In some instances, the individual has improvement in the one or more symptom within 14 days of administration of a composition described herein (e.g., Composition 1 or Composition 2). In some instances, the individual has improvement in the one or more sign and one or more symptom within 3 months of administration of a composition described herein (e.g., Composition 1 or Composition 2). In some instances, the individual has improvement in the one or more sign and one or more symptom within 1.5 months of administration of a composition described herein (e.g., Composition 1 or Composition 2). In some instances, the individual has improvement in the one or more sign and one or more symptom within 14 days of administration of a composition described herein (e.g., Composition 1 or Composition 2). In some instances, the individual has improvement in the one or more sign but not the one or more symptom within 14 days of administration of a composition described herein (e.g., Composition 1 or Composition 2). In some instances, the individual has deterioration of the one or more symptom within 14 days of administration of a composition described herein (e.g., Composition 1 or Composition 2). The individual may be provided another agent, such as an immunomodulator (e.g., an anti-biotic and/or an anti-inflammatory agent) or an analgesic (e.g., a local anesthetic), to reduce or lessen a symptom during the start treatment, such as during the first 14 days of treatment. In some instances, administration of the other agent is not necessary for more than 14 days. In some instances, the individual has improvement in the one or more symptom after the individual has improvement in the one or more sign. In some instances, the individual has improvement in the one or more symptom within 1.5 months of administration of a composition described herein (e.g., Composition 1 or Composition 2). In some instances, the individual has improvement in the one or more symptom within 3 months of administration of a composition described herein (e.g., Composition 1 or Composition 2). In some instances, the individual has improvement in the one or more sign within 14 days of administration of a composition described herein (e.g., Composition 1 or Composition 2) and improvement in the one or more symptom within 1.5 months or 3 months of administration of a composition described herein (e.g., Composition 1 or Composition 2).

In some embodiments, the improvement in the one or more sign described herein is determined by comparing an initial objective score described herein to a second objective score described herein. In some embodiments, the initial objective score is an objective measurement of a sign of an ocular indication. In some embodiments, the initial objective score is measured before the individual is provided an agent (e.g., a keratolytic agent) or composition described herein. In some embodiments, the initial objective score is a baseline measurement, such as a measurement taken before the individual is provided vehicle, placebo, or drug (e.g., selenium disulfide). In some embodiments, the initial objective score is a measurement determined for an individual that is not provided drug (e.g., selenium disulfide), such as an individual only provided a control (e.g., vehicle or placebo). In some embodiments, the second objective score is an objective measurement of a sign of an ocular indication. In some embodiments, the second objective score is measured after the individual is provided an agent (e.g., a keratolytic agent) or composition described herein. In some embodiments, another (a third, fourth, fifth, etc.) objective score is measured and evaluated to determine if treatment (e.g., amount, frequency, or the like) should continue, such objective score being compared to the initial objective score or any preceding objective score.

In some embodiments, the improvement in the one or more symptom described herein is determined by comparing an initial symptom score described herein to a second symptom score described herein. In some embodiments, a symptom for an ocular indication is evaluated to determine an initial symptom score. In some embodiments, the initial symptom score is measured before the individual is provided an agent (e.g., a keratolytic agent) or composition described herein. In some embodiments, the initial symptom score is a baseline measurement, such as a measurement taken before the individual is provided vehicle, placebo, or drug (e.g., selenium disulfide). In some embodiments, the initial symptom score is a measurement determined for an individual that is not provided drug (e.g., selenium disulfide), such as an individual only provided a control (e.g., vehicle or placebo). In some embodiments, a symptom for an ocular indication is evaluated to determine second symptom score. In some embodiments, the second symptom score is measured after the individual is provided an agent (e.g., a keratolytic agent) or composition described herein. In some embodiments, another (a third, fourth, fifth, etc.) symptom score is measured and evaluated to determine if treatment (e.g., amount, frequency, or the like) should continue, such symptom score being compared to the initial symptom score or any preceding symptom score.

In some instances, an objective score and a symptom score described herein (e.g., initial objective score, second objective score, initial symptom score, or second symptom score) are measured and/or determined concurrently. In some instances, an objective score and a symptom score described herein (e.g., initial objective score, second objective score, initial symptom score, or second symptom score) are measured and/or determined sequentially. In some instances, an objective score and a symptom score described herein (e.g., initial objective score, second objective score, initial symptom score, or second symptom score) are measured and/or determined at the same time. In some instances, an objective score and a symptom score described herein (e.g., initial objective score, second objective score, initial symptom score, or second symptom score) are measured and/or determined on the same day. In some instances, an objective score and a symptom score described herein (e.g., initial objective score, second objective score, initial symptom score, or second symptom score) are measured and/or determined around the same time. In some instances, an objective score and a symptom score described herein (e.g., initial objective score, second objective score, initial symptom score, or second symptom score) are measured and/or determined within several months (e.g., 3 months) of each other. In some instances, an objective score and a symptom score described herein (e.g., initial objective score, second objective score, initial symptom score, or second symptom score) are measured and/or determined within a week of each other. In some instances, an objective score described herein (e.g., initial objective score or second objective score) is measured and/or determined before a symptom score described herein (e.g., initial symptom score or second symptom score). In some instances, symptom score described herein (e.g., initial objective score or second objective score) is measured and/or determined about 1 day to 3 months after an objective score described herein (e.g., initial symptom score or second symptom score), such as within 3 months, within 2 months, within 1 month, or within 2 weeks of each other. In some instances, an objective score described herein (e.g., initial objective score or second objective score) is measured and/or determined after a symptom score described herein (e.g., initial symptom score or second symptom score). In some embodiments, an improvement in an objective score described herein is determined before an improvement in a symptom score described herein.

In some instances, one or more symptom hasn't improved by day 14. In some instances, one or more sign has improved by day 14. In some instances, one or more sign has improved by day 14, but one or more symptom has not yet improved (e.g., compare FIGS. 2, 4-7 to FIGS. 3, 14, 15A, 15D, 15E, and 15F). In some instances, the improvement of the one or more sign is provided by a morphological change to one or more meibomian gland of the individual. In some instances, improving the one or more sign through a morphological change provides symptom improvement, such as after continued use (e.g., for about 3 months) of a composition described herein. As discussed hereinabove and shown in the figures, improving a sign with a composition (e.g., Composition 1, Composition 2, or vehicle) described herein doesn't necessarily provide an (immediate) improvement in a symptom. Y et, after a period of time (e.g., at month 3, month 4.5, and/or month 6), an individual receiving a composition described herein (eventually) has a significant improvement in both signs and symptoms compared to baseline and control (vehicle or placebo). Contrarily, while an individual receiving vehicle may experience some improvements to signs, the individual fails to have any significant symptom improvement. As such, demonstrated herein are compositions and methods for improving meibomian gland function, thereby treating an ocular disorder described herein.

In some instances, treatment compliance is similar between drug and vehicle administration groups, such as described in Example 1.

In some embodiments, provided herein is a method comprising providing instructions to continue use of a composition described herein despite symptom degradation or lack of symptom improvement, such as within 3 months, 2 months, 1 month, or less of starting treatment. In some embodiments, provided herein is a method comprising providing instructions to continue use of a composition described herein despite symptom degradation or lack of symptom improvement within two weeks of starting treatment.

Provided in some embodiments herein is a kit comprising a package insert, the package insert providing instructions of a method described herein.

In some embodiments, the kit comprises a package insert, the package insert providing instructions of a method for treating an ocular disorder in an individual, the method comprising providing a keratolytic agent to or around the eye of the individual until an objective measure and symptom of the ocular disorder are improved.

In some embodiments, the kit comprises a package insert, the package insert providing instructions of a method for treating an ocular disorder in an individual, the method comprising (i) providing a keratolytic agent (e.g., selenium sulfide) to or around the eye of the individual for a first period of time, over which first period of time (e.g., 1-4 weeks) improvement in an objective measure of the ocular disorder is achieved; (ii) providing keratolytic agent (e.g., selenium sulfide) to or around the eye of the individual for a second period of time, over which second period of time (e.g., 2-26 weeks) improvement of a symptom of the ocular disorder is achieved; and (iii) providing keratolytic agent (e.g., selenium sulfide) to or around the eye of the individual, after which time (e.g., 1 month or more), improvement in the objective measure and/or symptom is maintained.

In some embodiments, any method described herein comprises providing a meibum enhancer, such as a keratolytic agent described herein, to the individual. In some instances, the meibum enhancer increases or improves the quality of meibum (secreted from the meibomian gland(s) of the individual). In some instances, the meibum enhancer increases or improves the quantity of meibum (secreted from the meibomian gland(s) of the individual). In some instances, the meibum enhancer increases or improves the quality and quantity of meibum (secreted from the meibomian gland(s) of the individual). In some instances, the meibum enhancer opens one or more meibomian gland of the individual, such as by breaking down a keratinized blockage in the one or more meibomian gland. In some instances, the meibum enhancer opens one or more meibomian gland of the individual and/or improves meibum quality and/or quantity (secreted from the meibomian gland(s) of the individual). In some embodiments, the meibum enhancer is a keratolytic agent, an immunomodulator, an immunomodulator (e.g., an anti-biotic and/or an anti-inflammatory agent), an analgesic (e.g., a local anesthetic), or any combination thereof.

In some embodiments, a composition described herein comprises a meibum enhancer.

In some embodiments, a composition described herein comprises a keratolytic agent. In some embodiments, the keratolytic agent is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, gluthatione, dithiothreitol, thiorphan, cysteamine, bucillamine, dimercaprol, 1,1-ethanedithiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamine, prosultiamine, thiram, lipoic acid, lenthionine, ajoene, allicin, gemopatrilat, thioethanol, thiophospholipid, thiocholesterol, 12-mercaptododecanoic acid, 23-(9-mercaptononyl)-3,6,9,12,15,18,21-heptaoxatricosanoic acid, and sulfanegen. In some embodiments, the keratolytic agent is selected from the group consisting of benzoyl peroxide, coal tar, dithranol, salicylic acid, selenium disulfide, alpha-hydroxy acid, urea, lactic acid, sodium thioglycolate, zinc pyrithione, or zinc L-pyrrolidone carboxylate.

In some embodiments, the keratolytic agent is selenium disulfide.

In some embodiments, the keratolytic agent (e.g., selenium sulfide) is administered to or around the eye of the individual at a concentration of at least about 0.01 wt. %. In some embodiments, the keratolytic agent (e.g., selenium sulfide) is administered to or around the eye of the individual at a concentration of at most about 30 wt. %. In some embodiments, the keratolytic agent (e.g., selenium sulfide) is administered to or around the eye of the individual at a concentration of about 0.01 wt. % to about 30 wt. %. In some embodiments, the keratolytic agent (e.g., selenium sulfide) is administered to or around the eye of the individual at a concentration of about 0.1 wt. % to about 20 wt. %. In some embodiments, the keratolytic agent (e.g., selenium sulfide) is administered to or around the eye of the individual at a concentration of about 0.1 wt. % to about 10 wt. %. In some embodiments, the keratolytic agent (e.g., selenium sulfide) is administered to or around the eye of the individual at a concentration of about 0.1 wt. % to about 5 wt. %. In some embodiments, the keratolytic agent (e.g., selenium sulfide) is administered to or around the eye of the individual at a concentration of about 0.1 wt. % to about 1 wt. %. In some embodiments, the keratolytic agent (e.g., selenium sulfide) is administered to or around the eye of the individual at a concentration of about 0.5 wt. %. In some embodiments, the keratolytic agent (e.g., selenium sulfide) is administered to or around the eye of the individual at a concentration of about 1.0 wt. %.

In some embodiments, Composition 1 is a selenium disulfide ointment/semi-solid formulation comprising 0.5 wt % of selenium disulfide.

In some embodiments, Composition 2 is a selenium disulfide ointment/semi-solid formulation comprising 1.0 wt % of selenium disulfide.

In some embodiments, a method described herein comprises providing a combination of keratolytic agents to or around the eye of the individual. In some embodiments, the method comprises providing a first keratolytic agent for a first period of time and a second keratolytic agent for a second period of time. In some embodiments, the first keratolytic agent and the second keratolytic agent are administered concurrently. In some embodiments, the first keratolytic agent and the second keratolytic agent are administered sequentially. In some embodiments, the first keratolytic agent is selenium disulfide. In some embodiments, the second keratolytic agent reduces a symptom described herein, such as pain (e.g., a local anesthetic).

In some instances, meibomian gland dysfunction (MGD) is a chronic and progressive condition associated with blockage of the meibomian glands and alteration in meibum quality, which can result in gland atrophy and loss from obstruction-associated back pressure. In some instances, MGD is associated with orifice plugging, duct obstruction and dilatation, gland atrophy and dropout, and qualitative changes in expressed secretions. In some instances, signs of MGD are characterized by abnormal keratin production and aggregation, altering the quality and quantity of meibum and leading to blocked meibomian glands. Meibomian gland orifices are located on the dermal portion of the tarsal plate of the upper and lower eyelid and are responsible for the production of meibum, a lipid-protein secretion that forms the outer component of the tear film. M eibum consists of hundreds of different wax and cholesteryl ester lipids and 90 different proteins that include various forms of keratin. In some instances, meibum provides tear film stability, provides ocular surface protection against microbial agents and organic matter, and reduces evaporation of the aqueous components of the tear film, such as playing an integral role in maintaining eye health. In some instances, MGD-associated abnormalities includes alterations of the tear film and vision quality, symptoms of eye irritation, clinically apparent inflammation, anterior blepharitis, contact lens discomfort (CLD), and ocular surface disease. In some instances, individuals with early-stage MGD are asymptomatic. In some instances, a treatment goal in MGD is ensuring a stable tear film for the ocular surface and preventing progressive glandular atrophy related to obstruction and associated back pressure.

In some instances, an individual provided herein has a best corrected visual acuity (BCVA) of 20/40 or better (Snellen equivalent), such as by using the logarithm of the minimum angle of resolution (LogMAR) in each eye.

In some instances, an individual provided herein has evidence of meibomian gland obstruction (e.g., MGS score of ≤12 for 15 glands of the lower eyelid) in at least one eye (e.g., both eyes).

In some instances, an individual provided herein reports dry eye signs and/or symptoms.

In some instances, an individual provided herein does not use (e.g., or discontinues use of) antihistamines or isotretinoin (e.g., for at least 1 month before treatment).

In some instances, an individual provided herein does not use (e.g., or discontinues use of) anti-inflammatory treatments for dry eye disease (e.g., cyclosporine ophthalmic emulsion (e.g., Restasis or Ikervis®) or lifitegrast ophthalmic solution (e.g., Xiidra)), such as for at least 3 months before treatment.

In some instances, an individual provided herein does not use (e.g., or discontinues use of) prescription medications for dry eye or MGD (e.g., antibiotics, corticosteroids, and non-steroidal anti-inflammatory drugs), such as for at least 2 weeks before treatment.

In some instances, an individual provided herein does not use (e.g., or discontinues use of) LipiFlow® or other lid-heating therapies.

In some instances, an individual provided herein does not use (e.g., or discontinues use of) lid-heating therapy.

In some instances, an individual provided herein does not use (e.g., or discontinues use of) therapeutic gland expression in either eye, such as within 6 months from treatment.

In some instances, an individual provided herein does not use (e.g., or discontinues use of) other MGD treatments (e.g., at-home warm compress therapy, eyelid hygiene, eyelid massage, and manual eyelid expression), such as for at least 2 weeks before treatment.

In some instances, an individual provided herein does not use (e.g., or discontinues use of) other topical ophthalmic preparations (e.g., artificial tear substitutes).

In some instances, an individual provided herein exhibits evidence of MGD, such as by a SPEED questionnaire (e.g., score ≥6), OSDI questionnaire (e.g., score ≥13 and <34), and TBUT (e.g., score <10 seconds in both eyes).

In some instances, an individual provided herein does not have uncontrolled ocular disease (e.g., except for MGD and dry eye disease/keratoconjunctivitis sicca) or uncontrolled systemic disease.

In some instances, an individual provided herein does not have glaucoma, ocular hypertension, intraocular pressure (IOP) in either eye (e.g., as determined by Goldman applanation tonometry), or planned insertion or removal of glaucoma filtration shunts or devices.

In some instances, an individual provided herein does not have a corneal abnormality or disorder that impacts normal spreading of the tear film (e.g., keratoconus, pterygia, scarring) or corneal integrity.

In some instances, an individual provided herein does not have a BCVA worse than 20/40 in either eye.

In some instances, an individual provided herein does not use (e.g., or has ever used) punctal plugs or anticipates using punctal plugs.

In some instances, an individual provided herein does not have a history of punctal cautery in either eye.

In some instances, an individual provided herein does not have keratoconjunctivitis sicca secondary to destruction of conjunctival goblet cells, such as occurs with vitamin A deficiency or scarring, such as that with cicatricial pemphigoid, alkali burns, Stevens-Johnson syndrome, trachoma, or irradiation.

In some instances, an individual provided herein does not have an active ocular infection (e.g., bacterial, viral, or fungal).

In some instances, an individual provided herein does not have corneal, conjunctival, or eyelid inflammation (e.g., allergic, vernal, or giant papillary conjunctivitis and mucous membrane pemphigoid).

In some instances, an individual provided herein has not had recent ocular surgery, trauma, herpes, or recurrent inflammation.

In some instances, an individual provided herein does not use contact lenses.

In some instances, an individual provided herein does not undergo periocular application of makeup or tattooing of the eyelids.

In some instances, an individual provided herein does not use scleral lenses or sealed compartment ocular frames (e.g., within 2 months of treatment).

In some instances, an individual provided herein is does not have eyelid abnormalities that affect normal eyelid function.

In some instances, an individual provided herein does not have a history of anterior segment surgery or trauma that could affect corneal sensitivity.

In some instances, an individual provided herein does not have a meibography score of 4.

In some instances, an individual provided herein does not have corneal staining ≥3 (between 33 and 100 dots) using the Oxford Scheme.

In some instances, an individual provided herein does not have a known allergy or sensitivity to fluorescein or lissamine green.

In some instances, an individual provided herein does not use medicated shampoos containing selenium.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Examples

### Example 1: Clinical E Valuation of Signs and Symptoms

The phase 2 clinical study described herein was designed to evaluate the safety and efficacy of Composition 1 (selenium disulfide 0.5 wt %) and Composition 2 (selenium disulfide 1.0 wt %), which are formulated as selenium disulfide ophthalmic ointments, over a six-month dosing period in individuals with signs and symptoms of MGD. The primary endpoints were evaluated at three months, and individuals were followed to six months to explore longer-term safety and efficacy durability of Composition 1 and Composition 2.

Composition 1 (selenium disulfide 0.5%) demonstrated clinically meaningful and statistically significant efficacy when compared to vehicle across multiple measures of the signs (MGYLS, MGS) and downstream effects (OSDI, SPEED, TBUT) of MGD. The response to treatment with Composition 1 (selenium disulfide 0.5%) on MGLYS was observed as early as Day 14 (after only four applications) and continued to show signs of improvement at Month 3, Month 4.5, and Month 6.

Composition 1 and Composition 2 are semi-solid ophthalmic ointments containing selenium sulfide.

### Methods

### Design

A multicenter, double-masked, vehicle-controlled, randomized, parallel group study carried out in 2 sequential cohorts-Cohort 1: sequential rising concentrations of selenium disulfide ointment/semi-solid drug (i.e., 0.1%, 0.5%, or 1.0%) and vehicle dosed twice-weekly and/or once daily in the evening; Expansion Cohort: parallel doses of placebo and two concentrations of selenium disulfide ointment/semi-solid drug (i.e., Composition 1 (selenium disulfide 0.5 wt %) or Composition 2 (selenium disulfide 1.0%)) dosed twice-weekly in the evening. The total duration of study is approximately 3.5 months (from screening to study completion) for Cohort 1 and is approximately 6.5 months (from screening to study completion) for the Expansion Cohort.

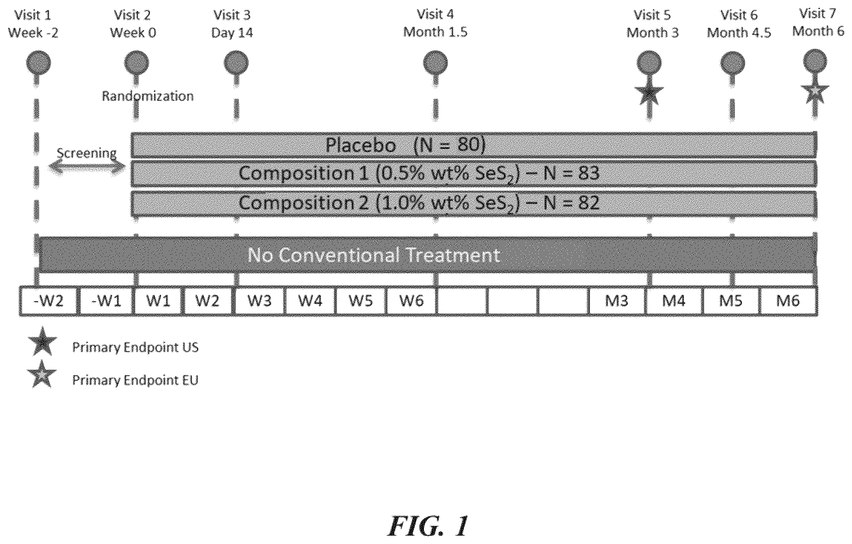

For the Expansion Cohort, individuals with MGD were randomly assigned in a 1:1:1 ratio to receive either a single concentration of placebo (vehicle) or selenium disulfide ointment/semi-solid drug (i.e., Composition 1 (selenium disulfide 0.5 wt %) or Composition 2 (selenium disulfide 1.0%)). All individuals were centrally assigned to randomized study treatment using an interactive web response system and were stratified by duration of MGD diagnosis (<5 or ≥5 years) and Meibomian Gland Secretion (MGS) score (<6, or ≥6 and ≤12) at baseline. Individuals were enrolled based on eligibility criteria, and all treatments for MGD or dry eye disease, including artificial tears (washout period). A screening visit was followed by a baseline visit 14 days later (qualification period). During the baseline visit, inclusion and exclusion criteria were confirmed. At the end of the qualification period individuals who still exhibit signs of MGD and who can comply with dosing instructions, such as by correctly dispensing the ointment using a dispensing aid and applying the demonstration medication (petrolatum white) to their lower eyelid, at the baseline visit were enrolled into a 6-month treatment period. The study design protocol for the expansion cohort is shown in FIG. 1.

In some instances, the primary endpoints (e.g., described elsewhere herein) of the study are evaluated using the following hierarchical approach to maintain the family-wise Type I error at 0.05%: (1) change from baseline to month 3 in MGYLS, comparing Composition 1 to placebo; (2) change from baseline to month 3 in OSDI total score, comparing Composition 1 0.5% to placebo; (3) change from baseline to month 3 in MGYLS, comparing Composition 2 to placebo; and (4) change from baseline to month 3 in OSDI total score, comparing Composition 2 to placebo.

### Eligibility Criteria

Inclusion and exclusion criteria are detailed in Table 1. Inclusion criteria included being aged 18 years or older and having evidence of meibomian gland obstruction in both eyes, a reported history of associated dry eye signs and symptoms within the past three months, and no significant glandular atrophy on meibography (<75%).

Exclusion criteria included a history or presence of any other ocular condition in either eye that would likely interfere with study data interpretation. Individuals with glaucoma, ocular hypertension, or elevated intraocular pressure in either eye or the planned insertion/removal of glaucoma filtration shunts/devices during the study were excluded. No corneal abnormality or disorder that impacted the normal spreading of the tear film (keratoconus, pterygia, scarring) or corneal integrity was allowed. The use of contact lenses, artificial tears, saline drops, or ocular lubricants was not permitted.

TABLE 1

| Inclusion Criteria | Exclusion Criteria |
|---|---|
| 1. Male or female, 18 years of age or older | 1. Uncontrolled ocular disease (except for |
| at screening. | MGD and dry eye |
| 2. Capable of understanding and willing to | disease/keratoconjunctivitis sicca) or |
| provide written informed consent and | uncontrolled systemic disease. |
| likely to complete the entire course of | 2. Individual had glaucoma, ocular |
| study according to instructions. | hypertension, or intraocular pressure (IOP) |
| 3. Written authorization for use and release | in either eye at Screening ≥24 mm Hg as |
| of health and research study information | determined by Goldman applanation |
| was obtained. | tonometry (or a COVID-19-compliant |
| 4. BCVA of 20/40 or better (Snellen | device as required by local governance) or |
| equivalent), using the logarithm of the | planned insertion/removal of glaucoma |
| minimum angle of resolution (LogMAR) | filtration shunts/devices during the study. |
| in each eye at the screening and baseline | 3. Corneal abnormality or disorder that |
| visits. | impacted normal spreading of the tear film |
| 5. Evidence of meibomian gland | (keratoconus, pterygia, scarring) or corneal |
| obstruction (MGS score of ≤12 for 15 | integrity. |
| glands of the lower eyelid) in both eyes | 4. BCVA worse than 20/40 in either eye at the |
| at the screening and baseline visits. | screening or baseline visit. |
| 6. Reported dry eye signs and symptoms | 5. Used punctal plugs, anticipated insertion |
| within the past 3 months. | during the study, or had a history of punctal |
| 7. Prior to screening, individuals were | cautery in either eye at any time prior to the |
| required to discontinue: use of systemic | screening visit or anticipated such a |
| antihistamines or isotretinoin for at least | procedure during the study. |
| 1 month; anti-inflammatory treatments | 6. Keratoconjunctivitis sicca secondary to |
| for dry eye disease (e.g., cyclosporine | destruction of conjunctival goblet cells as |
| ophthalmic emulsion [Restasis or | occurs with vitamin A deficiency or |
| Ikervis ®] or lifitegrast ophthalmic | scarring, such as that with cicatricial |
| solution [X iidra]) for at least 3 months; | pemphigoid, alkali burns, Stevens-Johnson |
| all other prescription medications used | syndrome, trachoma, or irradiation. |
| for dry eye or MGD (e.g., antibiotics, | 7. Keratoconjunctivitis sicca secondary to |
| corticosteroids, and nonsteroidal anti- | aqueous deficient dry eye disease. |
| inflammatory drugs) for at least 2 weeks; | 8. Active ocular infection (bacterial, viral, or |
| LipiFlow ® or other lid-heating therapy, | fungal) at screening or baseline. |
| meibomian gland probing, or therapeutic | 9. Corneal, conjunctival, or eyelid |
| gland expression in either eye within 6 | inflammation (including allergic, vernal, or |
| months prior to the screening visit; all | giant papillary conjunctivitis and mucous |
| other MGD treatments (e.g., at-home | membrane pemphigoid) that, in the |
| warm compress therapy, eyelid hygiene, | judgment of the investigator, could |
| eyelid massage, and manual eyelid | interfere with the study results or the ability |
| expression) for at least 2 weeks; and all | of individuals to complete the treatment |
| other topical ophthalmic preparations | period. |
| (including artificial tear substitutes) | 10. Recent (within 3 months prior to the |
| other than the study drops for at least 72 | screening visit) ocular surgery, trauma, |
| hours prior to screening visit. If artificial | herpes, or recurrent inflammation. |
| tear substitutes were used within 72 | 11. Contact lens use anticipated during the |
| hours of the screening visit, the visit was | study. |
| to be rescheduled. | 12. Periocular application of makeup during |
| 8. Evidence of MGD at the screening and | the study (e.g., mascara or eyeliner) that the |
| baseline visits: SPEED questionnaire | investigator feels could interfere with the |
| score ≥6, OSDI questionnaire score ≥13 | signs and symptoms of either MGD or |
| and <34, and TBUT <10 seconds in both | evaporative dry eye disease or tattooing of |
| eyes. | the eyelids. |
| 9. Demonstrated ability to follow dosing | 13. Used any type of scleral lenses or sealed |
| instructions at the baseline visit. | compartment ocular frames within 2 |
| 10. A negative pregnancy test result for all | months of the screening visit or planned to |
| women of childbearing potential at | use during the study. |
| screening. | 14. Used prohibited medications (topical, |
| 11. Women of childbearing potential were to | topical ophthalmic, systemic and/or |
| have a history of bilateral tubal ligation | injectable) during the appropriate pre-study |
| or use oral contraceptives, implants, | wash-out period and during the study. |
| injectables, transdermal patch, | 15. Unwilling to abstain from the use of |
| intrauterine device, or double barrier | systemic medications known to cause |
| contraceptive for birth control during the | dryness for the study duration that were not |
| study. If these methods of birth control | used on a stable dosing regimen for at least |
| did not apply, women of childbearing | 30 days prior to the baseline visit. |
| potential were to have a monogamous | 16. Unwilling to abstain from the use of |
| partner who had a vasectomy at least 3 | systemic or topical treatments for MGD or |
| months before screening. Complete | dry eye for the study duration (including |
| abstinence for 4 weeks before exposure | OTC artificial tears, ocular lubricants, or |
| to study medication, throughout the | dietary supplements known to impact |
| study, and for at least 4 weeks after the | ocular surface health). |
| last dose of study medication was | 17. Eyelid abnormalities that affect normal |
| acceptable for study inclusion. | eyelid function in either eye other than |
| | those caused by MGD. |
| | 18. Diagnosed with hepatitis C infection, |
| | human immunodeficiency virus (HIV) |
| | infection, sarcoidosis, amyloidosis, active |
| | tuberculosis, or graft versus host disease. |
| | 19. History of anterior segment surgery or |
| | trauma that could affect corneal sensitivity |
| | (e.g., cataract surgery or any surgery |
| | involving a limbal or corneal incision) in |
| | either eye within the 12 months prior to |
| | screening. |
| | 20. Planned anterior segment surgery (e.g., |
| | cataract surgery or any surgery involving a |
| | limbal or corneal incision) in either eye |
| | during the study period. |
| | 21. Meibography score at screening of 4 (>75% |
| | partial glands using the gestalt grading |
| | system). |
| | 22. Corneal staining ≥3 (between 33 and 100 |
| | dots) using the Oxford Scheme. |
| | 23. Schirmer's tear test without anesthesia ≤5 |
| | mm in either eye at the baseline visit. |
| | 24. Known allergy or sensitivity to fluorescein, |
| | lissamine green, or the study medication or |
| | its components. |
| | 25. Used medicated shampoos containing |
| | selenium (e.g., Selsun Blue, Exsel, Selsum, |
| | and Seleen) following the screening visit. |
| | 26. Individual was unlikely to follow study |
| | instructions or to complete all required |
| | study visits or had a condition or situation |
| | that, in the investigator's opinion, put the |
| | individual at significant risk, confounded |
| | the study results, or interfered significantly |
| | with the individual's participation in the |
| | study. |
| | 27. Individual was an employee at the |
| | investigational site or was related to any |
| | member of the study staff. |
| | 28. Pregnant, nursing, or females of |
| | childbearing potential that would not utilize |
| | adequate birth control measures. |
| | 29. Positive urine pregnancy test at screening. |
| | 30. Participation in another clinical trial |
| | involving a therapeutic drug or device |
| | within the past 30 days. |
| | 31. Enrolled in a previous stage of the current |
| | study or another study using Composition 1 |
| | or Composition 2. |

### Treatments

Study treatments consisted of Compound 1 (selenium disulfide 0.5 wt %), Composition 2 (selenium disulfide 1.0 wt %), or vehicle and were provided in identical 5 g multi-use opaque white tubes with a screw cap. Individuals and site staff were masked to treatment assignment. The randomized study drug was applied twice weekly by the individual using their washed index finger, such as immediately before sleep and on the lower eyelids of the study eye (the eye receiving active drug). Subsequent blinking transferred drug to the upper eyelid. To ensure consistent dosing between individuals and between applications, individuals were provided with a dispensing aid and trained on appropriate dispensing and application at the baseline visit.

### Assessments

Study visits occurred at screening (Visit 1, Day-14), baseline (Visit 2, Day 0), Day 14 (Visit 3), Month 1.5 (Visit 4), and Month 3 (Visit 5, primary endpoint). Best-corrected visual acuity, slit-lamp biomicroscopy (including eyelid margin erythema/telangiectasias), sodium fluorescein corneal staining (Oxford scale), lissamine green conjunctival staining (Oxford scale), and meibomian gland evaluation were assessed at all study visits. Meibomian gland evaluations were performed by the same investigator for all visits by a individual. Unanesthetized Schirmer tests were performed at screening, baseline, and Month 3, with intraocular pressure, ophthalmoscopy exam, and meibography assessed at screening and Month 3. The signs of MGD were measured by the number of meibomian glands yielding liquid secretion (MGYLS) and MGS scores; the symptoms of MGD were assessed using the Ocular Surface Disease Index (OSDI), Standard Patient Evaluation of Eye Dryness (SPEED), and tear break-up time (TBUT).

The signs of MGD were assessed by the number of open meibomian glands (i.e., MGYLS score) and quality of meibum (i.e., MGS score). The number of MGYLS is based on a technique for meibomian gland expression, where secretion in the lower eyelid of each eye was measured for five consecutive glands in each of three regions (temporal, central, and nasal). Expression was performed using a Meibomian Gland Evaluator on the 15 glands individually, with a binary score of 0 (none observed) or 1 (liquid observed) recorded following expression. The MGYLS is scored from 0-15, where lower scores indicate more severe disease. For the study, a MGYLS responder is someone who has an increase of ≥5 MGLYS from baseline, which lies between a score consistent with symptomatic disease (<4 responding glands) and non-symptomatic disease (≥6 responding glands).

The MGS is based on visual evidence of meibum quality. Using the same methodology as MGYLS assessment, secretion in the lower eyelid of each eye is measured on a total score scale of 0-45 per eye for which lower scores indicate more severe disease. Each gland is scored using a four-point scale where 0=no secretion, 1=inspissated/toothpaste consistency, 2=cloudy liquid secretion, and 3=clear liquid secretion. For the study, a MGS responder is someone who has a MGS score >12, indicating normal meibum quality.

The impacts of MGD were evaluated by symptoms (OSDI, SPEED) and TBUT. The OSDI questionnaire comprises 12 questions regarding ocular symptoms, environmental triggers, and vision-related functioning. The OSDI total score ranges 0-100, with higher scores representing greater disability; scores <13 represent normal, 13 to <23 represent mild, 23-33 represent moderate, and >33 represent severe dry eye disease. For the study, a OSDI responder is someone who has an OSDI total score <13, which is considered normal or asymptomatic for dry eye disease.

The SPEED total score is calculated based on occurrence, frequency, and severity of the four symptoms of eye dryness—grittiness or scratchiness, soreness or irritation, burning or watering, and eye fatigue—with the individual recording the time/occurrence of symptoms (at this visit, within past 72 hours, or within past 3 months). Derived from the frequency and severity scores across the four symptoms, SPEED total score ranges 0-28, with higher scores indicate increasing severity. For the study, scores from 0-4 are classified as ‘mild’ disease, 5-7 as ‘moderate’ disease, and ≥8 as ‘severe’ disease.

TBUT is the time, in seconds, taken for the first dry spot to appear on the cornea after a complete blink and was evaluated using a micropipette to instill 5 μL of 2% preservative-free sodium fluorescein based on triplicate measurements in each eye. The same investigator was required to perform these measurements for a individual during the study. Increased values demonstrate improvement, with TBUT responders defined as individuals with TBUT≥10 seconds, which is considered normal.

### Statistical Analysis

The co-primary efficacy endpoints were the change from baseline in signs of MGD, as measured by MGYLS, and symptoms of MGD, as measured by OSDI. The co-primary endpoints were evaluated using a hierarchical approach. For each endpoint, the subsequent endpoint was not evaluated unless the prior endpoint was significant at α=0.05 to control for family-wise Type I error and remove any adjustment for multiplicity. The hierarchical approach was change from baseline to Month 3 in: A) MGYLS, comparing Composition 1 to vehicle; B) OSDI total score, comparing Composition 1 to vehicle; C) MGYLS, comparing Composition 2 to vehicle; and D) OSDI total score, comparing Composition 2 to vehicle.

Efficacy analyses were performed on available data from the intent-to-treat (ITT) population, which consisted of all randomized individuals. The safety population comprised individuals who were randomized and received at least one dose of study treatment. Co-primary endpoints were analyzed separately using an ANCOVA model with terms for baseline value, treatment, and analysis center. Additional secondary and exploratory efficacy endpoints were analyzed in a similar manner. Categorical variables are summarized by sample size (N), frequency count and percent, and analyzed using Cochran-Mantel-Haenszel to evaluate the differences between treatments, controlling for disease duration category and baseline MGS score category.

### Results

### Disposition and Baseline Characteristics

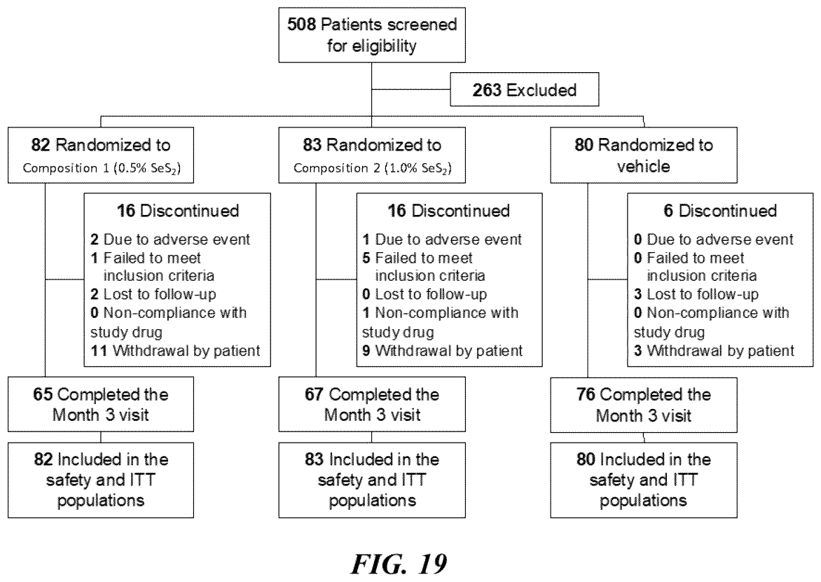

A total of 245 individuals with signs and symptoms of MGD were randomized and included in the ITT and safety populations (FIG. 19); 35 (14.3%) individuals discontinued the study before the Month 3 visit, with the rates of three-month completion being 79.3% (n=65/82) for Composition 1, 80.7% (n=67/83) for Composition 2, and 95.0% (n=76/80) for vehicle. Over three months of exposure, individuals were expected to administer 24 doses of study drug; a total of 196 (94.2%) individuals were compliant (80-125% of doses taken) with study drug administration, with similar overall compliance across all three treatment groups.

Baseline demographics and clinical characteristics were similar across treatment arms, with signs and symptoms consistent with that for an MGD individual population, see Table 2. Individuals were largely white (72.2%), female (66.5%), and had a mean age of 53.2 years. The duration of MGD, according to self-reporting at baseline, was >5 years in 64.5% of individuals, with 57.1% of individuals reporting a baseline MGS score between 6 and 12, inclusive.

TABLE 2

| | | Composition 1 | Composition 2 | |
|---|---|---|---|---|
| | | 0.5% | 1.0% | Vehicle |
| | | (N = 82) | (N = 83) | (N = 80) |
| Age (years) | Mean (SD) | 52.1 (16.9) | 55.6 (17.9) | 51.9 (18.47) |
| | Range | 18-80 | 20-93 | 20-97 |
| Gender, n (%) | Male | 31 (37.8) | 27 (32.5) | 24 (30.0) |
| | Female | 51 (62.2) | 56 (67.5) | 56 (70.0) |
| Race, n (%) | White | 57 (69.5) | 64 (77.1) | 56 (70.0) |
| | Asian | 16 (19.5) | 10 (12.0) | 21 (26.3) |
| | Black | 3 (3.7) | 3 (3.6) | 1 (1.3) |
| | Pacific Islander | 0 | 1 (1.2) | 0 |
| | Other | 6 (7.3) | 5 (6.0) | 2 (2.5) |
| Duration of MGD, n (%) | <5 years | 29 (35.4) | 30 (36.1) | 28 (35.0) |
| | ≥5 years | 53 (64.6) | 53 (63.9) | 52 (65.0) |
| MGYLS score | Mean (SD) | 1.7 (1.35) | 1.9 (1.36) | 1.8 (1.34) |
| MGS score, n (%) | <6 | 38 (46.3) | 33 (39.8) | 34 (42.5) |
| | ≥6 and ≤12 | 44 (53.7) | 50 (60.2) | 46 (57.5) |
| MGS Score at Baseline | Mean (SD) | 5.7 (2.75) | 6 (3.02) | 6 (2.78) |
| OSDI total score | Mean (SD) | 25 (7.46) | 24 (5.98) | 25 (6.72) |

### Co-Primary Endpoints

Composition 1 met both co-primary endpoints, see Table 3. There was significantly greater improvement in MGYLS scores from baseline to Month 3 with 0.5% than vehicle, indicating that treatment with the compositions herein resulted in more open meibomian glands compared to vehicle (FIG. 2). In addition, there was significantly greater improvement from baseline to Month 3 in mean OSDI total score with 0.5% compared to vehicle, indicating that Composition 1 resulted in greater symptom relief than vehicle (FIG. 3). For both co-primary endpoints, there was a numerical improvement with Composition 2 over vehicle that did not reach statistical significance. At month 6, the 0.5% treatment showed statistical significance vs vehicle in MGYLS (FIG. 2) and OSDI (FIG. 3). Specifically, Composition 1 achieved statistically significant improvements vs vehicle for both co-primary endpoints, MGYLS and OSDI score, at Month 3, which continued through to Month 6. The 1.0% treatment showed a significant improvement in MGYLS (FIG. 2) and no statistically significant difference in OSDI (FIG. 3).

TABLE 3

| | | |
|---|---|---|
| Composition 1 | Composition 2 | |
| 0.5% | 1.0% | Vehicle |
| (N = 82) | (N = 83) | (N = 80) |

Co-primary Endpoints

| | | | |
|---|---|---|---|
| Change from baseline in MGYLS score | 4.2 (0.36) | 3.2 (0.37) | 2.4 (0.34) |
| at Month 3, LS mean (SE) | | | |
| P value vs baseline | <0.0001 | <0.0001 | <0.0001 |
| P value vs vehicle | 0.0004 | 0.14 | |
| Change from baseline in OSDI total score at | −7.3 (1.26) | −6.1 (1.29) | −3.8 (1.22) |
| Month 3, LS mean (SE) | | | |
| P value vs baseline | <0.0001 | <0.0001 | 0.0028 |
| P value vs vehicle | 0.0438 | 0.18 | |

Secondary Endpoints
Change from baseline in MGYLS score

| | | | |
|---|---|---|---|
| Day 14, LS mean (SE) | 1.6 (0.22) | 1.6 (0.22) | 0.8 (0.21) |
| P value vs baseline | <0.0001 | <0.0001 | 0.0002 |
| P value vs vehicle | 0.0080 | 0.0071 | |
| Month 1.5, LS mean (SE) | 2.9 (0.29) | 2.8 (0.28) | 1.8 (0.28) |
| P value vs baseline | <0.0001 | <0.0001 | <0.0001 |
| P value vs vehicle | 0.0046 | 0.0078 | |

Change from baseline in OSDI total score

| | | | |
|---|---|---|---|
| Day 14, LS mean (SE) | −2.4 (1.28) | −1.7 (1.29) | −2.3 (1.27) |
| P value vs baseline | 0.0635 | 0.1867 | 0.0684 |
| P value vs vehicle | 0.97 | 0.73 | |
| Month 1.5, LS mean (SE) | −5.0 (1.26) | −3.3 (1.27) | −3.3 (1.23) |
| P value vs baseline | 0.0002 | 0.0125 | 0.0084 |
| P value vs vehicle | 0.33 | 0.97 | |

MGYLS responder rate

| | | | |
|---|---|---|---|
| Day 14 (mean %) | 7.8% | 9.8% | 1.3% |
| P value vs vehicle | 0.10 | 0.0334 | |
| Month 1.5 (mean %) | 27.2% | 28.4% | 5.4% |
| P value vs vehicle | 0.0012 | 0.0006 | |
| Month 3 (mean %) | 45.7% | 28.8% | 14.7% |
| P value vs vehicle | 0.0005 | 0.096 | |

OSDI total score responder rate

| | | | |
|---|---|---|---|
| Day 14 (mean %) | 17.7% | 23.5% | 16.3% |
| P value vs vehicle | 0.53 | 0.52 | |
| Month 1.5 (mean %) | 31.5% | 27.1% | 23.8% |
| P value vs vehicle | 0.53 | 0.70 | |
| Month 3 (mean %) | 46.9% | 38.4% | 28.2% |
| P value vs vehicle | 0.0199 | 0.39 | |

Change from baseline in SPEED score

| | | | |
|---|---|---|---|
| Day 14, LS mean (SE) | −2.3 (0.46) | −1.4 (0.46) | −2.2 (0.46) |
| P value vs baseline | <0.0001 | 0.0028 | <0.0001 |
| P value vs vehicle | 0.88 | 0.21 | |
| Month 1.5, LS mean (SE) | −2.6 (0.50) | −2.1 (0.52) | −2.3 (0.49) |
| P value vs baseline | <0.0001 | 0.0002 | <0.0001 |
| P value vs vehicle | 0.61 | 0.74 | |
| Month 3, LS mean (SE) | −4.3 (0.462) | −4.1 (0.450) | −2.8 (0.44) |
| P value vs baseline | <0.0001 | <0.0001 | <0.0001 |
| P value vs vehicle | 0.018 | 0.031 | |

Change from baseline in MGS score

| | | | |
|---|---|---|---|
| Day 14, LS mean (SE) | 4.0 (0.51) | 4.1 (0.51) | 2.1 (0.50) |
| P value vs baseline | <0.0001 | <0.0001 | <0.0001 |
| P value vs vehicle | 0.0081 | 0.0035 | |
| Month 1.5, LS mean (SE) | 7.1 (0.75) | 7.0 (0.71) | 4.2 (0.72) |
| P value vs baseline | <0.0001 | <0.0001 | <0.0001 |
| P value vs vehicle | 0.0048 | 0.0056 | |
| Month 3, LS mean (SE) | 10.5 (0.91) | 8.1 (0.88) | 6.0 (0.84) |
| P value vs baseline | <0.0001 | <0.0001 | <0.0001 |
| P value vs vehicle | 0.0003 | 0.075 | |

MGS responder rates

| | | | |
|---|---|---|---|
| Day 14 (mean %) | 19.9% | 18.5% | 7.6% |
| P value vs vehicle | 0.0380 | 0.095 | |
| Month 1.5 (mean %) | 49.7% | 46.1% | 27.8% |
| P value vs vehicle | 0.0073 | 0.0375 | |
| Month 3 (mean %) | 68.7% | 50.7% | 44.4% |
| P value vs vehicle | 0.0069 | 0.98 | |

Change from baseline in TBUT

| | | | |
|---|---|---|---|
| Month 3, LS mean (SE) seconds | 2.21 (0.29) | 1.53 (0.33) | 0.52 (0.29) |
| P value vs baseline | <0.0001 | <0.0001 | 0.0786 |
| P value vs vehicle | <0.0001 | 0.0187 | |

### Secondary Analyses

Secondary analyses revealed that treatment with Composition 1 provided a clinically meaningful and statistically significant increase in the number of open meibomian glands that expressed liquid with individuals classified as MGYLS responders. Additionally, individuals treated with Composition 1 demonstrated a statistically significant decrease in OSDI total scores and a greater proportion were asymptomatic for disease on the instrument by Month 3. Many individuals treated with a composition described herein were considered asymptomatic for disease (OSDI <13) at Month 3 (Composition 1=47%, p=0.0199 vs vehicle; Composition 2=38%, p>0.05 vs vehicle; vehicle=28%), which was sustained for Composition 1 and improved for Composition 2 at Month 6 (Composition 1=48%, p=0.0333 vs vehicle; Composition 2=50% p=0.0205 vs vehicle; vehicle=30%). Composition 1 provided greater improvement when compared to vehicle on the symptoms associated with MGD, as measured by SPEED. Other secondary and exploratory efficacy evaluations support that the clinical effects related to treatment with Composition 1 directly increased as a function of time on drug across multiple indicators.

Mean MGYLS scores at baseline were similar between the study and non-study eyes across all three treatment groups. There was a significantly greater improvement in MGYLS scores from baseline to Day 14 and to Month 1.5 in both Composition 1 and Composition 2 treatment groups compared to vehicle (Table 3; FIG. 2). At Months 1.5 and 3, a significantly greater percentage of individuals treated with Composition 1 than vehicle were MGYLS responders (i.e., ≥5-gland increase from baseline), and at Day 14 and Month 1.5, significantly more individuals treated with Composition 1 than vehicle were MGYLS responders (Table 3; FIG. 5). The improvement in MGYLS scores from baseline in both Composition 1 and Composition 2 treatment groups compared to vehicle continued to improve with continued use through 6-months (FIG. 2 and FIG. 5). At all timepoints, the percentage of responders with Composition 1 or Composition 2 was numerically higher than with vehicle.

At baseline, no individuals were classified as ‘normal’ or asymptomatic for disease per the OSDI (score <13) as all individuals were diagnosed with symptomatic MGD. Analysis of OSDI total score responder rates at Month 3 found significantly more individuals were asymptomatic for disease in the Composition 1 group compared to vehicle; at all three timepoints, the responder rates in both treatment groups were numerically higher than in the vehicle group (Table 3; FIG. 10). Also, analysis of OSDI total score responder rates at Month 6 found significantly more individuals were asymptomatic for disease in the Composition 1 and Composition 2 groups compared to vehicle (FIG. 10).

There was greater improvement from baseline in MGS score at all three timepoints in the Composition 1 and Composition 2 treatment groups (Table 3). The Composition 1 treatment group showed significantly greater improvement in MGS scores compared to vehicle at all timepoints. Composition 2 demonstrated significantly greater improvement in MGS scores at Day 14 and Month 1.5 and numerically greater improvement at Month 3 compared to vehicle. At all three timepoints in the clinical trial, the percentage of MGS responders (i.e., individuals with a ‘normal’ value of MGS >12) in both treatment groups was higher than in the vehicle group (Table 3; FIG. 7). Significantly more individuals in the Composition 1 treatment group had ‘normal’ quality meibum at all timepoints, with numerically greater MGS responders in the Composition 2 group at each timepoint compared to vehicle. Individuals continued to demonstrate normal meibum at Month 6 (74%, p=0.0017 for Composition 1; 63%, p=0.1273 for Composition 2; 48% for vehicle). By Month 6, significantly more patients treated with a composition described herein were asymptomatic for disease (OSDI <13: 48%, p=0.0333 for Composition 1; 50%, p=0.0205 for Composition 2; 30% for vehicle).

Evaluation of downstream effects of MGS and MGYLS improvements are seen in the TBUT, which reached significant improvements from baseline at all timepoints for the Composition 1 and Composition 2 treatment groups and significant difference compared to vehicle at Month 1.5 for Composition 1 and Month 3, 4.5, and 6 for both treatment groups (Table 3; FIG. 8). A significantly higher percentage of individuals treated with Composition 1 or Composition 2 achieved normal TBUT of ≥10 seconds (i.e., TBUT responders) at Month 3 compared with vehicle (FIG. 9).

### Safety and Tolerability

Composition 1 and Composition 2 were moderately safe and well tolerated, with 137 of 245 (55.9%) individuals across all treatment groups reporting a treatment-emergent adverse event (TEAE) (65 non-ophthalmic events, 118 ophthalmic events), and 86.1% of those reporting a TEAE reporting an ophthalmic TEAE (Table 4). Individuals in both Composition 1 and Composition 2 treatment groups rated most (93%) ophthalmic TEAEs as mild to moderate in severity. TEAEs reported in ≥5% of any treatment group were application-site pain (0.5%, n=14 (17.1%); 1.0%, n=13 (15.7%); and vehicle, n=0, respectively), increased lacrimation (11.0%, 1.2%, 0%), superficial punctate keratitis (9.8%; 8.4%; 1.3%), corneal staining (6.1%, 8.4%, 1.3%), eye pain (6.1%; 7.2%; 1.3%), eye irritation (4.9%, 6.0%, 2.5%), application-site irritation (2.4%, 6.0%, 0%) and eye inflammation (3.7%, 9.6%, 1.3%).

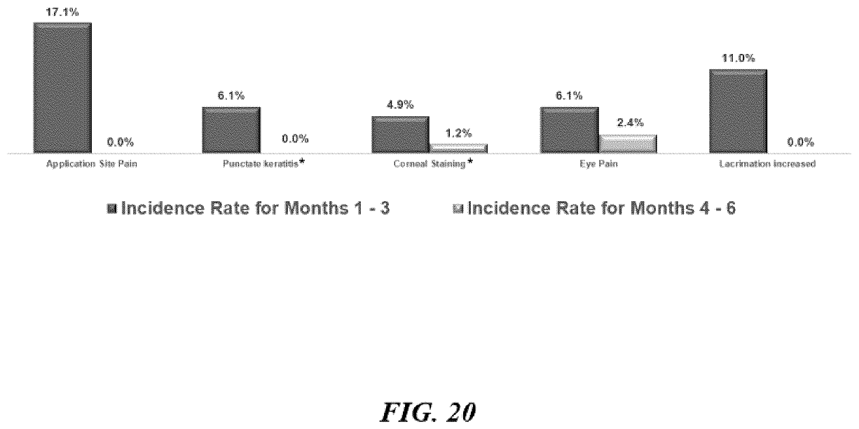

There were two (2.4%) individuals in the Composition 1 group, 1 (1.2%) in the Composition 2 group, and no individuals in the vehicle group who discontinued the study due to an adverse event. There were five treatment-emergent SAEs reported by four individuals. The five events included pericarditis, thyroid mass, pneumonia, post procedural hemorrhage, and nephrolithiasis. Moreover, at month 6, most (96%) TEAEs in the Composition 1 group were mild to moderate in severity and only two additional subjects (2.4%) discontinued for adverse events. As shown in FIG. 20, a significant (e.g., ≥5%) reduction of treatment-emergent adverse events (TEAEs) was observed during months 4-6 of treatment. In particular, rates of application site pain, punctate keratitis, and increase in lacrimation dropped completely (to 0%) with Composition 1 during months 4-6, further demonstrating that continuing use of a therapeutically effective concentrations of selenium disulfide (e.g., to the eyelid margin) minimizes adverse effects while also providing therapeutic benefit. Note, the * in FIG. 20 indicates that the test is defined as associated with an increase in corneal staining of ≥2 grades. The SAEs reported during this study were not considered to be study drug related by the investigator and were all non-ophthalmic. There were no deaths reported during the study.

TABLE 4

| | Composition 1 | Composition 2 | |
|---|---|---|---|
| | 0.5% | 1.0% | Vehicle |
| | (N = 82) | (N = 83) | (N = 80) |
| Any TEAEs, n (%) | 54 (65.9%) | 61 (73.5%) | 22 (27.5%) |
| Any ophthalmic TEAEs (in either eye), n (%) | 47 (57.3%) | 57 (68.7%) | 14 (17.5%) |
| Any possibly, probably, or certainly related | 42 (51.2%) | 50 (60.2%) | 10 (12.5%) |
| TEAEs, n (%) | | | |

TEAEs reported in ≥5% of individuals, n (%)

| | | | |
|---|---|---|---|
| Application-site pain | 14 (17.1%) | 13 (15.7%) | 0 |
| Lacrimation increased | 9 (11.0%) | 1 (1.2%) | 0 |
| Superficial punctate keratitis* | 5 (6.1%) | 6 (7.2%) | 1 (1.3%) |
| Vital dye staining cornea present*^{†} | 4 (4.9%) | 7 (8.4%) | 1 (1.3%) |
| Eye pain | 5 (6.1%) | 6 (7.2%) | 1 (1.3%) |
| Eye irritation | 4 (4.9%) | 5 (6.0%) | 2 (2.5%) |
| Application-site irritation | 2 (2.4%) | 5 (6.0%) | 0 |
| Any serious TEAEs, n (%)^{‡} | 1 (1.2%) | 1 (1.2%) | 2 (2.5%) |
| Study drug withdrawal due to TEAEs, | 11 (13.4%) | 9 (10.8%) | 1 (1.3%) |
| n (%)^{§} | | | |

This study was designed to establish the maximum tolerated dose, and the results confirmed In some instances, Composition 1 (0.5% Selenium disulfide) is a dose beyond which no further benefit is anticipated following twice-weekly, chronic dosing. In some instances, Composition 2 (1.0% selenium disulfide) is numerically better than vehicle, according to changes from baseline in MGYLS and OSDI scores and could potentially demonstrate statistical superiority over vehicle with an increased sample size or duration of treatment. In some instances, preclinical data for selenium disulfide determined that the effect on sebum production in sebaceous cells demonstrates a non-linear dose response where a lower effect was observed at higher concentrations.

In some instances, the data herein demonstrated the safety and tolerability of Composition 1 and Composition 2 over the three to six months of treatment, finding no major safety or tolerability concerns. In some instances, the data supports the conclusion that twice weekly bedtime dosing improves tolerability. In some instances, the data described herein demonstrates compositions described herein have strong efficacy, restore gland function, and improve symptoms (e.g., in individuals with MGD), such as over a 6 month treatment period.

We claim:

1. A method for determining therapeutic efficacy of a treatment for an ocular disease or disorder in an individual, the method comprising:
a) evaluating meibum quality, meibum quantity, number of open meibomian glands, or a combination thereof of the individual to determine an initial objective score, the initial objective score being an initial MGS score or an initial MGYLS score;
b) providing a therapeutic agent to or around the eye of the individual;
c) evaluating meibum quality, meibum quantity, number of open meibomian glands, or a combination thereof of the individual to determine a second objective score, the second objective score being a second MGS score or a second MGYLS score;
d) determining the second MGS score or the second MGYLS score is an improvement over the initial MGS score or the second MGYLS score,
wherein, the ocular disease or disorder is meibomian gland dysfunction or dry eye disease.

2. The method of claim 1, wherein the second objective score is an improvement over the initial objective score and the improvement is determined after providing the therapeutic agent to or around the eye of the individual.

3. The method of claim 1, wherein the initial objective score is the initial MGS score.

4. The method of claim 1, wherein the initial objective score is the initial MGYLS score.

5. The method of claim 1, wherein the ocular disease or disorder is meibomian gland dysfunction.

6. The method of claim 5, wherein the initial objective score is the initial MGS score.

7. The method of claim 6, wherein the second objective score the second MGS score, and the second MGS score is an improvement of about 10 or more.

8. The method of claim 5, wherein the initial objective score is the initial MGYLS score.

9. The method of claim 6, wherein the second objective score the second MGYLS score, and the second MGYLS score is an improvement of at least 3.

10. The method of claim 1, wherein the ocular disease or disorder is dry eye disease.

11. The method of claim 10, wherein the initial objective score is the initial MGS score.

12. The method of claim 11, wherein the second objective score the second MGS score, and the second MGS score is an improvement of about 10 or more.

13. The method of claim 10, wherein the initial objective score is the initial MGYLS score.

14. The method of claim 13, wherein the second objective score the second MGYLS score, and the second MGYLS score is an improvement of at least 3.

15. A method for determining therapeutic efficacy of a treatment for meibomian gland dysfunction (MGD) in an individual, the method comprising:
a) evaluating meibum quality, meibum quantity, number of open meibomian glands, or a combination thereof of the individual to determine an initial objective score, the initial objective score being an initial MGS score or an initial MGYLS score;
b) providing a therapeutic agent to or around the eye of the individual;
c) evaluating meibum quality, meibum quantity, number of open meibomian glands, or a combination thereof of the individual to determine a second objective score, the second objective score being a second MGS score or a second MGYLS score;
d) determining the second MGS score or the second MGYLS score is an improvement over the initial MGS score or the second MGYLS score.

16. The method of claim 15, wherein the initial objective score is the initial MGS score.

17. The method of claim 16, wherein the second objective score the second MGS score, and the second MGS score is an improvement of about 10 or more.

18. The method of claim 15, wherein the initial objective score is the initial MGYLS score.

19. The method of claim 16, wherein the second objective score the second MGYLS score, and the second MGYLS score is an improvement of at least 3.

20. A method for determining therapeutic efficacy of a treatment for meibomian gland dysfunction (MGD) in an individual, the method comprising (i) identifying a first and second score, the first and second score being a first and a second MGS score or a first and a second MGYLS score, and (ii) determining if the second score is an improvement over the first score; wherein the first and second score were determined by:
a) evaluating meibum quality, meibum quantity, number of open meibomian glands, or a combination thereof of the individual to determine the first score;
b) providing a therapeutic agent to or around the eye of the individual; and
c) following providing the therapeutic agent to or around the eye of the individual, evaluating meibum quality, meibum quantity, number of open meibomian glands, or a combination thereof of the individual to determine the second score.
